(19) United States
(12) Patent Application Publication
(10) Pub. No.: US 2008/0031815 A1
(43) Pub. Date: Feb. 7, 2008

(54) Pet imaging of vascular endothelial growth factor receptor (VEGFR), compositions for VEGF cancer imaging, and methods of VEGF cancer imaging

(76) Inventors: Xiaoyuan Chen, Union City, CA (US); Weibo Cai, Sunnyvale, CA (US); Sanjiv S. Gambhir, Portola Valley, CA (US); Hui Wang, Dalian (CN); Kai Chen, Mountain View, CA (US); Martin Rodriguez-Porcel, Rochester, MN (US); Juergen K. Willmann, Buchenbach (DE)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/881,384

(22) Filed: Jul. 26, 2007

Related U.S. Application Data
(60) Provisional application No. 60/833,569, filed on Jul. 27, 2006.

(51) Int. Cl. A61K 49/00; A61K 51/08; C07K 14/00; G21H 5/02

(52) U.S. Cl. 424/9.1; 250/303; 530/350

(57) ABSTRACT

Briefly described, embodiments of this disclosure include polypeptides, in particular a labeled VEGF protein, kits for imaging, methods for imaging tissue, methods of diagnosing the presence in a tissue of one or more of precancerous cells, cancerous cells, tumor cells, and cells related to ischemic or hypoxic related diseases, methods of monitoring the progress in a tissue of the presence of one or more of precancerous cells, cancerous cells, tumor cells, and cells related to ischemic or hypoxic related diseases, and the like.

### CROSS-REFERENCE TO RELATED APPLICATION

This application claims priority to U.S. provisional applications entitled, “PET IMAGING OF VASCULAR ENDOTHELIAL GROWTH FACTOR RECEPTOR (VEGFR), COMPOSITIONS FOR VEGF CANCER IMAGING, AND METHODS OF VEGF CANCER IMAGING,” having Ser. No. 60/833,569, filed on Jul. 27, 2006, which is entirely incorporated herein by reference.

### STATEMENT REGARDING FEDERALLY SPONSORED RESEARCH OR DEVELOPMENT

This invention was made with Government support under contracts CA 114747 and CA082214 awarded by the National Institutes of Health. The Government has certain rights in this invention.

### BACKGROUND

Positron emission tomography (PET) is a diagnostic examination that involves the acquisition of physiologic images based on the detection of radioactive decay from the emission of positrons. In particular, PET is a nuclear medicine medical imaging technique that produces a three-dimensional image or map of functional processes in the body. Positrons are antiparticles emitted from a radioactive tracer isotope administered to the patient. The subsequent images of the human body developed with this technique are used to evaluate a variety of diseases.

A short-lived radioactive isotope that decays by emitting a positron, chemically incorporated into a molecule (e.g., a metabolically active molecule), is injected into the living subject (e.g., usually into blood circulation). There is a waiting period while the molecule becomes concentrated in tissues of interest; then the subject is placed in the imaging scanner. The short-lived isotope decays, emitting a positron. After travelling up to a few millimeters the positron annihilates with an electron, producing a pair of annihilation photons (similar to gamma rays) moving in opposite directions. These are detected when they reach a scintillator material in the scanning device, creating a burst of light that is detected by photomultiplier tubes. The technique depends on simultaneous or coincident detection of the pair of photons: photons that do not arrive in pairs (e.g., within a few nanoseconds) are ignored.

Because annihilation photons are always emitted 180° apart, it is possible to localize their source to a straight-line in space. Using statistics collected from tens-of-thousands of coincidence events, a map of the origin of the annihilation photons in the body can be plotted. The resulting PET scan shows the tissues in which the molecular probe has become concentrated. These results can be interpreted by a nuclear medicine physician or radiologist and assist in the patient's diagnosis and treatment plan. PET is used heavily in clinical oncology (medical imaging of tumors and the search for metastases) and in human brain and heart research.

PET imaging is most useful in combination with anatomical imaging, such as CT (computer tomography). Thus, modern PET scanners are now available with integrated high-end multi-detector-row CT scanners. Since the two scans can be performed simultaneously, time is saved. Additionally, the two concurrent images allows for a direct comparison of the anatomy depicted in the CT scan with the area of increased radiolabeled molecular probe uptake seen on the PET scan.

Angiogenesis, the growth of new blood vessels, is an important natural process occurring in the body in both normal physiological processes (e.g., wound healing, pregnancy) and in pathological disease processes (e.g., tumorigenesis, stroke, rheumatoid arthritis). During adulthood, most blood vessels remain quiescent and angiogenesis occurs only in the cycling ovary and in the placenta during pregnancy. However, when angiogenic growth factors are produced in excess of angiogenesis inhibitors, such as is found in, for example, cancer and psoriasis, endothelial cells are stimulated to proliferate. One of the most extensively studied angiogenesis-related signaling pathways is the VEGF/VEGFR interactions. VEGF, a potent mitogen in embryonic and somatic angiogenesis, plays a pivotal role in both normal vascular tissue development and in many disease processes. The VEGF family is composed of seven members with a common VEGF homology domain: VEGF-A, VEGF-B, VEGF-C, VEGF-D, VEGF-E, VEGF-F, and placenta growth factor. VEGF-A is a dimeric, disulfide-bound glycoprotein existing in at least seven homodimeric isoforms, consisting of 121, 145, 148, 165, 183, 189, or 206 amino acids. Besides the difference in molecular weight, these isoforms also differ in their biological properties (e.g., the ability to bind to cell surface heparin sulfate proteoglycans).

VEGF signaling is mediated largely via two endothelium-specific receptor tyrosine kinases, Flt-1/FLT-1 (VEGFR-1) and Flk-1/KDR (VEGFR-2). Both VEGFRs are largely restricted to vascular endothelial cells and all VEGF-A isoforms bind to both VEGFR-1 and VEGFR-2. It is now generally accepted that VEGFR-1 is critical for physiologic and developmental angiogenesis and its function depends on stages of development, physiologic and pathologic status, and the cell types in which it is expressed. VEGFR-2 is the major mediator of the mitogenic, angiogenic, and permeability-enhancing effects of VEGF. In clinical studies, over-expression of VEGF and/or VEGFRs has been associated with poor prognosis. Thus, agents that prevent VEGF-A binding to its receptors, antibodies that directly block VEGFR-2, and small molecules that inhibit the growth factor signaling activity of VEGFR-2, are all currently under active development. The critical role of VEGF-A in cancer progression has been highlighted by the approval of the first anti-angiogenic humanized anti-VEGF monoclonal antibody bevacizumab (Avastin®; Genentech) for first line treatment of metastatic carcinomas. Development of VEGF- and VEGFR-targeted molecular imaging probes could serve as a new paradigm for the assessment of anti-angiogenic therapeutics and further our understanding of the role that VEGF/VEGFR plays in the pathogenesis of angiogenesis-related diseases.

### SUMMARY

Briefly described, embodiments of this disclosure include polypeptides, in particular a labeled VEGF protein, kits for imaging, methods for imaging tissue, methods of diagnosing the presence in a tissue of one or more of precancerous cells, cancerous cells, tumor cells, and cells related to ischemic or hypoxic related diseases, methods of monitoring the progress in a tissue of the presence of one or more of precancerous cells, cancerous cells, tumor cells, and cells related to ischemic or hypoxic related diseases, and the like.

One exemplary polypeptide, among others, includes: a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label.

One exemplary kit for imaging, among others, includes: a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and directions for use.

One exemplary method for imaging tissue, among others, includes: contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label, and imaging the tissue with a PET imaging system.

One exemplary method of diagnosing the presence in a tissue of one or more of tissue containing precancerous cells, tissue containing cancerous cells, tissue containing tumor cells, and tissue containing cells related to ischemic or hypoxic related diseases, among others, includes: contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and imaging the tissue with a PET imaging system.

One exemplary method of monitoring the progression in a tissue of the presence of one or more of tissue containing precancerous cells, c tissue containing ancerous cells, tissue containing tumor cells, and tissue containing cells related to ischemic or hypoxic related diseases, among others, includes: contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE}(SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and imaging the tissue with a PET imaging system.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

The patent or application file contains at least one drawing executed in color. Copies of this patent or patent application publication with color drawing(s) will be provided by the Office upon request and payment of the necessary fee.

FIG. 1 illustrates the cell binding assay and functional assay. FIG. 1(a) illustrates a cell binding assay of VEGF₁₂₁ and DOTA-VEGF₁₂₁ using PAE/KDR cells (VEGF₁₂₁ protein (SEQ ID No: 1). IC₅₀ values are 1.02 nM and 1.66 nM for VEGF₁₂₁ and DOTA-VEGF₁₂₁, respectively. FIG. 1(b) illustrates a functional assay of VEGF₁₂₁ and DOTA-VEGF₁₂₁. Tubulin was used as loading control.

FIG. 2 illustrates a microPET imaging of ⁶⁴Cu-DOTA-VEGF₁₂₁ in U87MG tumor-bearing mice. FIG. 2(a) illustrates a serial microPET scans of large and small U87MG tumor-bearing mice injected with ⁶⁴Cu-DOTA-VEGF₁₂₁. Mice injected with ⁶⁴Cu-DOTA-VEGF₁₂, 30 minutes after injection of VEGF₁₂₁ are also shown (denoted as “Small Tumor+Block”). FIG. 2(b) illustrates a two-dimensional whole body projection at 16 h post-injection of the three mice shown in FIG. 2(a). The tumors are pointed to by arrows.

FIG. 3 illustrates microPET and biodistribution results. FIG. 3(a) illustrates a comparison of ⁶⁴Cu-DOTA-VEGF₁₂₁ uptake in small and large U87MG tumor (n=3 per group). FIG. 3(b) illustrates time activity curves of kidney, liver, and muscle (n=6). FIG. 3(c) illustrates biodistribution of ⁶⁴Cu-DOTA-VEGF₁₂₁ in mice previously injected with 100 μg of VEGF₁₂₁, at 23 h p.i. FIG. 3(d) illustrates a comparison of ⁶⁴Cu-DOTA-VEGF₁₂₁ uptake in small U87MG tumor previously injected with/without 100 μg of VEGF₁₂₁ (n=3 per group). FIG. 3(e) illustrates a comparison of the quantification results obtained from biodistribution and microPET studies (n=3). *: P <0.05; **: P<0.01.

FIG. 4 illustrates an immunofluorescence staining of VEGFR1, VEGFR2, and CD31 for kidney, small U87MG tumor, and large U87MG tumor.

FIG. 5 illustrates a microvessel density (MVD) analysis and Western blot. FIG. 5(A) illustrates a MVD analysis of the small and large U87MG tumor. **: P<0.01. FIG. 5(B) illustrates a Western blot of VEGFR2 in the small and large U87MG tumor. Tubulin was used as a loading control.

FIG. 6 illustrates a cardiac functional assessment using ¹⁸F-FDG-PET and high resolution ultrasound (at a frequency of 30 MHz). FIGS. 6A and 6B illustrate ¹⁸F-FDG-PET of sham operated (FIG. 6A) and animals after myocardial infarction (MI, FIG. 6B). Sham operation did not induce any FDG defect, while MI was associated with a medium size defect in the anterolateral wall (arrow). FIGS. 6C and 6D illustrate an M-mode Ultrasound at the level of the mid ventricle in sham operation (FIG. 6C) and MI animals (FIG. 6D). Cardiac function was assessed 10 days post-operatively by calculation of myocardial fractional shortening (see text for formula). After MI, there was akinesis of the anterolateral wall and a significant decrease in fractional shortening, compared to sham operated animals.

FIG. 7 illustrates the myocardial origin of ⁶⁴Cu-DOTA-VEGF₁₂₁ PET signal after myocardial infarction (MI). FIG. 7 (top) illustrates a representative co-registered images of microCT (left), microPET (right) in an MI animal, and fused microPET-microCT image (center) clearly demonstrating that the ⁶⁴Cu-DOTA-VEGF₁₂₁ signal detected with microPET corresponds to the anterolateral myocardium (microPET and fused images, red arrow), and clearly separated from the intercostals muscle layer (microCT image, white arrow). There is also increased uptake in the area of the surgical wound (microPET image, green arrow). FIG. 7 (bottom) illustrates representative images of ⁶⁴Cu-DOTA-VEGF₁₂₁ (left), ¹⁸F-FDG (right), and ⁶⁴Cu-DOTA-VEGF₁₂₁-¹⁸F-FDG fused image (middle). FDG scan shows that coronary artery ligation resulted in a lack of ¹⁸F-FDG uptake (yellow arrow), and that the uptake of ⁶⁴Cu-DOTA-VEGF₁₂₁ occurs in areas supplied by the ligated coronary artery (blue arrow). Fusion of both scans results in complementation of ¹⁸F-FDG and ⁶⁴Cu-DOTA-VEGF₁₂₁ signals. There is also increased uptake in the area of the surgical wound (green arrow).

FIG. 8 (top) illustrates representative images at baseline (left), sham-operated animals (center) and in animals after MI (right), showing the difference in myocardial uptake in MI animals compared to baseline and sham animals. The red arrow shows the ⁶⁴Cu-DOTA-VEGF₁₂₁ signal from the myocardium (seen only in MI animals) and yellow arrows shows the ⁶⁴Cu-DOTA-VEGF₁₂₁ signal from the surgical wound (muscle layer), which is present in both sham operated and MI animals. FIG. 8 (bottom) illustrates the quantification of ⁶⁴Cu-DOTA-VEGF₁₂₁ after MI over time, expressed in % ID/gram of tissue. The ⁶⁴Cu-DOTA-VEGF₁₂₁ uptake was highest at day 3 post-operatively (compared to baseline, day-3), and continues to be elevated until day 17 post-operatively. *p<0.05 compared to baseline.

FIG. 9A illustrates an autoradiograph of 30 μm myocardial slices of both sham-operated (left) and MI animals (right) after injection of ⁶⁴Cu-DOTA-VEGF₁₂₁, showing that the increased signal in the anterolateral wall of the LV of MI animals, while no activity is detected in the sham group. Red arrows point to the area affected by the ligated artery (anterolateral wall).

FIG. 9B (top) illustrates an autoradiograph of 20 μm myocardial slices of both sham operated (right) and MI animals (right) after exposure to ¹²⁵I-VEGF₁₆₅, demonstrating an increased uptake of ¹²⁵I-VEGF₁₆₅ in MI animals. FIG. 9B (bottom) illustrates slides contiguous to those shown above (for both groups) have been co-incubated with unlabeled VEGF₁₂₁ (in addition to ¹²⁵I-VEGF₁₆₅), demonstrating that when VEGF receptors are blocked, the uptake of ¹²⁵I-VEGF₁₆₅ diminishes significantly (VEGF₁₆₅ protein (SEQ ID No: 4)). These results suggest that the increased probe uptake observed after MI is due to a specific binding of the probe to VEGF receptors, further supporting the concept that MI is associated with an increase in the number of VEGF receptors.

FIG. 10 illustrates immunofluorescence staining for VEGF receptor-1 (left) and -2 (right) in sham-operated and MI animals (at days 3, 10, 17, and 24 post MI induction). MI is associated with a marked increase in VEGF receptor 1 and 2 immunostaining, which was higher than sham animals. The VEGF receptor expression is higher at day 3 and diminishes over time, similar to what it is observed with microPET imaging.

FIG. 11 illustrates graphs that demonstrate binding assays of VEGF₁₂₁ and VEGF_{NSL} (SEQ ID No: 9) to VEGFR-2 in FIG. 11(a) and VEGFR-1 in FIG. 11(b). VEGF_{NLS} (SEQ ID No: 9) has decreased affinity to VEGFR-2 (IC₅₀ value of 10 μmol/l) compared with wild type VEGF₁₂₁ (SEQ ID No: 1) (IC₅₀ value of 2.9 nmol/l) and has decreased affinity, to a lesser extent, to VEGFR-1 (IC₅₀ value of 12 nmol/l). Measurements were done in triplicates. FIG. 12 illustrates serial ⁶⁴Cu-VEGF₁₂₁ uptake in four male C57BL/6J mice (supine position) at day 8 (FIG. 12(a)), day 15 (FIG. 12(b)), day 22 (FIG. 12(c)), and day 29 (FIG. 12(d)) after left femoral arterial ligation. On representative coronal thick-slab maximum intensity projections of microPET data obtained at 1 h after intravenous injection of radiotracer, a progressive decrease in ⁶⁴Cu-VEGF₁₂₁ uptake (as expressed as mean values of % ID/g) in the left ischemic hindlimb (arrow) over successive postoperative time points is demonstrated. Right contralateral non-ischemic hindlimbs were used as negative control and show low background ⁶⁴Cu-VEGF₁₂₁ uptake. Note radiotracer uptake in liver (L) and left kidney (K; right kidney can not be differentiated from liver on coronal projections).

FIG. 13 illustrates the uptake values of serial ⁶⁴Cu-VEGF₁₂₁ PET imaging of mice with and without treadmill exercise training at days 8, 15, 22, and 29 after surgery. ⁶⁴Cu-VEGF₁₂₁ uptake (expressed as % ID/g) was highest at day 8 after surgery and gradually decreased over the following three weeks. ⁶⁴Cu-VEGF₁₂₁ uptake was higher in ischemic hindlimbs of exercised mice compared to non-exercised mice. ⁶⁴Cu-VEGF₁₂₁ uptake was not significantly increased in contralateral non-ischemic hindlimbs of exercised mice. (Columns, mean; bars, ± standard deviation.)

FIG. 14(a) illustrates micrographs (at ×200) show representative immunohistochemical staining (VEGFR1, red; VEGFR2, green) of frozen hindlimb muscle tissue slices at day 8 and day 29 after surgery. VEGFR2 was overexpressed in ischemic hindlimb muscle tissue compared to contralateral control non-ischemic hindlimb muscle tissue, whereas VEGFR1 staining of ischemic hindlimb muscle tissue was not different compared to contralateral control hindlimb muscle tissue. FIG. 14(b) illustrates CD31 staining (green color) of ischemic hindlimb muscle tissue (at day 8 after surgery) suggests colocalization of CD31 and VEGFR2 on endothelial cells of muscle vessels.

FIG. 15 illustrates a representative western blot analysis of VEGFR2 protein expression in ischemic and non-ischemic (control) hindlimb muscle tissue of exercised (+training) and non-exercised mice at days 8, 15, 22, and 29 after femoral artery ligation. The expression of α-tubulin was used as a loading control.

FIG. 16 illustrates the stroke in the rat brain, confirmed by T2-weighed MRI and ¹⁸F-FDG PET, was delineated by ⁶⁴Cu-DOTA-VEGF₁₂₁.

FIG. 17 illustrates a schematic representation of VEGF₁₂, and its mutant VEGF_{DEE} (mutated at the 63, 64, and 67 positions).

FIG. 18 illustrates the cell binding assay and functional assay. FIG. 18(a) illustrates a cell binding assay of VEGF₁₂₁ (SEQ ID No: 1) and VEGF_{DEE} (SEQ ID No: 8) using PAE/VEGFR-1 (PAE/FLT-1) cells. IC₅₀ values are 4.2 nM and 78.1 nM for VEGF₁₂₁, and VEGF_{DEE}, respectively. FIG. 18(b) illustrates a cell binding assay of VEGF₁₂₁ and VEGF_{DEE} using PAE/KDR cells. IC₅₀ values are 2.9 nM and 11.7 nM for VEGF₁₂₁ and VEGF_{DEE}, respectively. FIG. 18c illustrates a cell binding assay of VEGF₁₂₁ and DOTA-VEGF₁₂₁ using PAE/KDR cells. IC₅₀ values are 2.9 nM and 5.0 nM for VEGF₁₂₁, and DOTA-VEGF₁₂₁, respectively. FIG. 18(d) illustrates a cell binding assay of VEGF_{DEE} and DOTA-VEGF_{DEE} using PAE/KDR cells. IC₅₀ values are 11.7 nM and 10.3 nM for VEGF_{DEE} and DOTA-VEGF_{DEE}, respectively. FIG. 18e illustrates a functional assay of VEGF₁₂₁, VEGF_{DEE}, and their DOTA conjugates using PAE/KDR cells. All four proteins are functionally active at concentrations greater than 5 nM. Tubulin was used as a loading control.

FIG. 19 illustrates microPET imaging studies of 4T1 tumor-bearing mice. FIG. 19(a) illustrates a serial microPET scans of 4T1 tumor-bearing mice injected intravenously with 5-8 MBq of ⁶⁴Cu-DOTA-VEGF₁₂₁ or ⁶⁴Cu-DOTA-VEGF_{DEE}. Mice co-injected with 200 μg of VEGF₁₂₁ are also shown (denoted as “+Block”). Coronal slices containing the tumors (arrowheads) are shown. FIG. 19(b) illustrates coronal, sagittal and axial slices containing the kidney (arrowheads) at 4 h p.i. of ⁶⁴Cu-DOTA-VEGF₁₂₁ or ⁶⁴Cu-DOTA-VEGF_{DEE}. Note the different scale in FIG. 19(a) and FIG. 19(b).

FIG. 20 illustrates a ROI analysis of the microPET scans. FIG. 20(a) illustrates a comparison of ⁶⁴Cu-DOTA-VEGF₁₂₁ and ⁶⁴Cu-DOTA-VEGF_{DEE} uptake in the 4T1 tumor. FIG. 20(b) illustrates a comparison of ⁶⁴Cu-DOTA-VEGF₁₂₁ and ⁶⁴Cu-DOTA-VEGF_{DEE} uptake in the kidney. FIG. 20(c) illustrates a comparison of ⁶⁴Cu-DOTA-VEGF₁₂₁ and ⁶⁴Cu-DOTA-VEGF_{DEE} uptake in the liver. Data shown represents mean ±SD (n=3 per group). **: P<0.01.

FIG. 21 illustrates a biodistribution of ⁶⁴Cu-DOTA-VEGF₁₂₁ and ⁶⁴Cu-DOTA-VEGF_{DEE} in 4T1 tumor-bearing mice at 4 h post-injection. Data shown represents mean ±SD (n=3 per group). **: P<0.01.

FIG. 22 illustrates immunofluorescence staining of VEGFR-1 and VEGFR-2 in the kidney, liver, 4T1 tumor, and muscle. For VEGFR-1 staining, frozen tissue slices (5 μm thick) were stained with rabbit anti-mouse VEGFR-1 primary antibody and a Cy3-conjugated donkey anti-rabbit secondary antibody. For VEGFR-2 staining, the tissue slices were stained with a rat anti-mouse VEGFR-2 primary antibody and a Cy3-conjugated donkey anti-rat secondary antibody.

FIG. 23 illustrates the VEGF₃₋₁₂₁-Avi fusion gene structure, where Avi-tag is fused to the C-terminus of VEGF₃₋₁₂₁.

FIG. 24 illustrates the argrose electrophoresis of PCR products.

FIG. 25 illustrates the enodnuclease restriction enzyme digestion of VEGF₃₋₁₂₁-Avi.

FIG. 26 illustrates the ybbR tag, a short (11-residue) peptide (DSLEFIASKLA), which was found to be an efficient substrate for Sfp-catalyzed protein labeling, thereby replacing the full-length PCP or ACP domain for the construction of smaller fusion of the target protein.

FIG. 27A illustrates a general scheme for constructing N-terminus fusion protein. FIG. 27B illustrates a general scheme for constructing C-terminus fusion protein.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Embodiments of the present disclosure will employ, unless otherwise indicated, techniques of biochemistry, molecular biology, medicine, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

### DEFINITIONS

In describing and claiming the disclosed subject matter, the following terminology will be used in accordance with the definitions set forth below.

Vascular endothelial growth factor (VEGF)-A plays a central role in both normal vascular tissue development and tumor neovascularization. VEGF-A is an angiogenic protein released by a variety of tumor cell lines and found to be expressed in various human tumors.

Through alternative splicing of RNA, VEGF may exist as at least seven different molecular isoforms, having 121 amino acids (VEGF₁₂₁, SEQ ID No: 1), 145 amino acids (VEGF₁₄₅, SEQ ID No: 2), 148 amino acids (VEGF₁₄₈, SEQ ID No: 3), 165 amino acids (VEGF₁₆₅, SEQ ID No: 4), 183 amino acids (VEGF₁₈₃, SEQ ID No: 5), 189 amino acids (VEGF₁₈₉, SEQ ID No: 6), or 206 amino acids (VEGF₂₀₆, SEQ ID No: 7). It should be noted that VEGF_{XXX} refers to VEGF_{XXX} protein, and it is understood that reference to VEGF_{XXX} referes to VEGF_{XXX} protein. These isoforms differ not only in their molecular weight but also in their biological properties, such as the ability to bind to cell surface heparin sulfate proteoglycans.

VEGF₁₂₁ is a soluble, non-heparin-binding variant that exists in solution as a disulfide-linked homodimer containing the full biological and receptor-binding activity of the larger variants.

As used herein, the terms “antibody” and “antibodies” can include, but are not limited to, monoclonal antibodies, multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (e.g., anti-Id antibodies to antibodies of the disclosure), and epitope-binding fragments of any of the above. In particular, antibodies include immunoglobulin molecules and immunologically active fragments of immunoglobulin molecules (e.g., molecules that contain an antigen binding site). Immunoglobulin molecules can be of any type (e.g., IgG, IgE, IgM, IgD, IgA and IgY), class (e.g., IgG1, IgG2, IgG3, IgG4, IgA1 and IgA2), or subclass. The antibodies may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. The antibodies may be monospecific, bispecific, trispecific, or of greater multispecificity.

As used herein, “humanized” describes antibodies wherein some, most or all of the amino acids outside the CDR regions are replaced with corresponding amino acids derived from human immunoglobulin molecules. In one embodiment of the humanized forms of the antibodies, some, most, or all of the amino acids outside the CDR regions have been replaced with amino acids from human immunoglobulin molecules but where some, most, or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody would retain a similar antigenic specificity as the original antibody.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of this disclosure and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly where the biologically functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also refers to the degree of sequence relatedness between polypeptide as determined by the match between strings of such sequences. “Identity” and “similarity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073, (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present invention.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution (including conservative and non-conservative substitution), or insertion, and wherein said alterations may occur at the amino- or carboxy-terminus positions of the reference polypeptide sequence or anywhere between those terminal positions, interspersed either individually among the amino acids in the reference sequence, or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein. Nonsense-suppressing tRNAs are used to incorporate an unnatural amino acid at the desired site in a protein and chemical means to attach the unnatural amino acid to the nonsense-suppressing tRNA. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

By “administration” is meant introducing an embodiment of the present disclosure into a subject. The preferred route of administration is intravenous. However, any route of administration, such as oral, topical, subcutaneous, peritoneal, intraarterial, inhalation, vaginal, rectal, nasal, introduction into the cerebrospinal fluid, or instillation into body compartments can be used. In accordance with the present disclosure, “an effective amount” of embodiments of the present disclosure is defined as an amount sufficient to yield an acceptable result. An effective amount of the sensor of the present disclosure may be administered in more than one injection. The effective amount of embodiments of the present disclosure can vary according to factors such as the degree of susceptibility of the individual, the age, sex, and weight of the individual, idiosyncratic responses of the individual, the dosimetry, and the like. Optimization of such factors is well within the level of skill in the art.

As used herein, the term “organelle” refers to cellular membrane-bound structures such as the chloroplast, mitochondrion, and nucleus. The term “organelle” includes natural and synthetic organelles.

As used herein, the term “non-nuclear organelle” refers to any cellular membrane bound structure present in a cell, except the nucleus.

As used herein, the term “host” or “organism” includes humans, mammals (e.g., cats, dogs, horses, etc.), living cells, and other living organisms. A living organism can be as simple as, for example, a single eukaryotic cell or as complex as a mammal. Typical hosts to which embodiments of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. Additionally, for in vitro applications, such as in vitro diagnostic and research applications, body fluids and cell samples of the above subjects will be suitable for use, such as mammalian (particularly primate such as human) blood, urine, or tissue samples, or blood, urine, or tissue samples of the animals mentioned for veterinary applications.

General Discussion

The present disclosure includes labeled VEGF (e.g., VEGF₁₂₁) proteins, labeled VEGF proteins included in compositions and pharmaceutical compositions, methods of using the labeled VEGF proteins, methods of diagnosing and/or targeting cancer and/or tumors, kits for imaging, kits for diagnosing and/or targeting cancer and/or tumors, methods of diagnosing and/or targeting ischemic or hypoxic related diseases (e.g., ischemia, infarct, and stroke), kits for diagnosing and/or targeting ischemic or hypoxic related diseases, and the like. In addition, the present disclosure includes compositions used in and methods relating to non-invasive positron emission tomography (PET) imaging of cancer in vivo.

In particular, the composition and/or pharmaceutical composition includes a labeled VEGF₁₂₁ protein (SEQ ID No: 1) that is used in PET imaging in vivo. For example, the labeled VEGF₁₂₁ protein can include a label linked (e.g., directly and/or indirectly) to the VEGF₁₂₁ protein with a chelator (e.g., a macrocyclic chelator such as 1,4,7,10-tetraazadodecane-N,N′,N″, N′″-tetraacetic acid (DOTA)), where the label is a PET label (e.g., radioisotope labels such as ¹⁸F and ⁶⁴Cu). In an embodiment, the labeled VEGF protein includes, but is not limited to, ⁶⁴Cu-DOTA-VEGF₁₂₁ protein and, ¹⁸F-VEGF₁₂₁ protein. In another embodiment, the labeled VEGF protein includes ⁶⁴Cu-DOTA-VEGF_{DEE} Mutant (SEQ ID No: 8). Additional details are provided in the Examples.

Although gamma emitters currently are more readily available and have longer half-lives relative to positron emitting radionuclides, PET cameras allow electronic rather than mechanical collimation of incoming photons by recording the coincidence of simultaneous pairs of annihilation photons (511 keV per photon) at opposite detectors. The sensitivity of PET is at least 1-2 orders of magnitude better than single photon imaging systems. The acquisition of higher count statistics is particularly valuable for detecting the fewest possible cells per unit volume with the least amount of radioactivity. The spatial/temporal resolution of PET is also significantly higher than single photon emission computed tomography (SPECT), which in turn allows dynamic scans and small lesion detection by PET. Embodiments of the present disclosure have been successfully applied to acquire PET images of both subcutaneous and orthotopic brain tumor models.

As mentioned above, the labeled VEGF proteins can be used to diagnose and/or target cancer and/or tumors as well as diagnose and/or target ischemic or hypoxic related diseases.

“Cancer”, as used herein, shall be given its ordinary meaning, as a general term for diseases in which abnormal cells divide without control. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

Benign tumors have less of a tendency to invade and are less likely to metastasize. Brain tumors spread extensively within the brain but do not usually metastasize outside the brain. Gliomas are very invasive inside the brain, even crossing hemispheres. They do divide in an uncontrolled manner, though. Depending on their location, they can be just as life threatening as malignant lesions. An example of this would be a benign tumor in the brain, which can grow and occupy space within the skull, leading to increased pressure on the brain.

It should be noted that cancerous cells, cancer, and tumors are often used interchangeably in the disclosure.

Diseases with ischemic or hypoxic mechanisms (e.g., ischemic or hypoxic related diseases) can be sub-classified into peripheral diseases and cerebral ischemia. Examples of such general diseases involving ischemic or hypoxic mechanisms include coronary heart disease, angina pectoris, myocardial infarction (stenosis of coronary arteries), cardiac insufficiency, myocarditis, pericarditis, perimyocarditis, congenital heart disease, shock, peripheral vascular disease (ischemia/infarction of extremities), stenosis of renal arteries, diabetic retinopathy, thrombosis associated with malaria, native cardiac valvular dysfunction, complications associated with prosthetic heart valves, anemias, hypersplenic syndrome, emphysema, lung fibrosis, and pulmonary edema. Examples of cerebral ischemia disease include stroke (as well as hemorrhagic stroke), cerebral microangiopathy (small vessel disease), intrapartal cerebral ischemia, cerebral ischemia during/after cardiac arrest or resuscitation, cerebral ischemia due to intraoperative complications (e.g., cerebral ischemia during carotid surgery), chronic cerebral ischemia due to stenosis of blood-supplying arteries to the brain, sinus thrombosis or thrombosis of cerebral veins, and cerebral vessel malformations.

Labeled VEGF

In general, embodiments of the labeled VEGF protein include a VEGF protein, portions thereof, mutants thereof, and varients thereof. The VEGF protein can include, but is not limited to, VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25).

In addition, it should be noted that other VEGF protein sequences can be mutated in a similar manner as VEGF_{DEE} by adding one of the following to the N-terminus or C-terminus: Avi-tag (SEQ ID No: 26), ybbR-tag (SEQ ID No: 27), FGE-tag (SEQ ID No: 28), and Cys-tag (SEQ ID No: 29). For example, the following proteins can be created by the addition of the sequences noted above to a particular VEGF protein: Avi-VEGF₁₄₅, VEGF₁₄₅-Avi, ybbR-VEGF₁₄₅, VEGF₁₄₅-ybbR, FGE-VEGF₁₄₅, VEGF₁₄₅-FGE, Cys-VEGF₁₄₅, VEGF₁₄₅-Cys, Avi-VEGF₁₄₈, VEGF₁₄₈-Avi, ybbR-VEGF₁₄₈, VEGF₁₄₈-ybbR, FGE-VEGF₁₄₈, VEGF₁₄₈-FGE, Cys-VEGF₁₄₈, VEGF₁₄₈-Cys, Avi-VEGF₁₆₅, VEGF₁₆₅-Avi, ybbR-VEGF₁₆₅, VEGF₁₆₅-ybbR, FGE-VEGF₁₆₅, VEGF₁₆₅-FGE, Cys-VEGF₁₆₅, VEGF₁₆₅-Cys, Avi-VEGF₁₈₃, VEGF₁₈₃-Avi, ybbR-VEGF₁₈₃, VEGF₁₈₃-ybbR, FGE-VEGF₁₈₃, VEGF₁₈₃-FGE, Cys-VEGF₁₈₃, VEGF₁₈₃-Cys, Avi-VEGF₁₈₉, VEGF₁₈₉-Avi, ybbR-VEGF₁₈₉, VEGF₁₈₉-ybbR, FGE-VEGF₁₈₉, VEGF₁₈₉-FGE, Cys-VEGF₁₈₉, VEGF₁₈₉-Cys, Avi-VEGF₂₀₆, VEGF₂₀₆-Avi, ybbR-VEGF₂₀₆, VEGF₂₀₆-ybbR, FGE-VEGF₂₀₆, VEGF₂₀₆-FGE, Cys-VEGF₂₀₆, and VEGF₂₀₆-Cys.

In an embodiment, the VEGF protein is the VEGF₁₂₁ protein. It should be noted that notation of these proteins in the text without the sequence identity number should be understood to refer to the proteins and sequence identity number mentioned above. Additional details regarding the VEGF₁₂₁ protein are described in the Examples.

The term “mutant” is employed broadly to refer to a protein that differs in some way from a reference wild-type protein, where the protein may retain biological properties of the reference wild-type (e.g., naturally occurring) protein, or may have biological properties that differ from the reference wild-type protein. The term “biological property” of the subject proteins includes, but is not limited to, biological interactions in cancer and/or ischemic or hypoxic related diseases, in vivo and/or in vitro stability (e.g., half-life), and the like. Mutants can include single amino acid changes (point mutations), deletions of one or more amino acids (point-deletions), N-terminal truncations, C-terminal truncations, insertions, and the like. Mutants can be generated using standard techniques of molecular biology.

In almost all the literature reports, VEGFR imaging was achieved using VEGF-A based tracers. All VEGF-A isoforms bind to both VEGFR-1 and VEGFR-2. The kidney is usually a dose-limiting organ because it has high VEGFR-1 expression, which can absorb VEGF-A based tracers. As described below, it has been found that the uptake of ⁶⁴Cu-DOTA-VEGF₁₂₁ in the kidneys was mainly due to VEGFR-1 binding (and some renal clearance), while the tumor uptake was mainly related to VEGFR-2 expression. The high renal uptake thus may limit its future clinical applications. Embodiments of the present disclosure describe the importance of developing VEGFR-2 specific mutants.

One example of VEGFR-2 specific mutant is VEGF_{DEE} (SEQ ID No. 8). Based upon the reported crystal structures VEGF/VEGFR-2 and VEGF/VEGFR-1 complexes (Cell 1997; 91(5):695-704, which is include herein by reference), amino acid residues 16-25 in helix α1, 61-67 in loop β3-β4, and 103-106 in β7 may be involved in VEGF/VEGFR-1 but not VEGF/VEGFR-2 interactions. It is thus possible to have VEGF variants that have amino acid residues modified in these regions to confer VEGFR-2 specificity. Typically VEGFR-2 specific mutants by changing amino acid sequences in these regions may be developed and optimized by using phage display technique.

In particular, mutants can include protein sequences selected from the following: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25).

In addition, mutants can include protein sequences selected from the following: Avi-VEGF₁₄₅, VEGF₁₄₅-Avi, ybbR-VEGF₁₄₅, VEGF₁₄₅-ybbR, FGE-VEGF₁₄₅, VEGF₁₄₅-FGE, Cys-VEGF₁₄₅, VEGF₁₄₅-Cys, Avi-VEGF₁₄₈, VEGF₁₄₈-Avi, ybbR-VEGF₁₄₈, VEGF₁₄₈-ybbR, FGE-VEGF₁₄₈, VEGF₁₄₈-FGE, Cys-VEGF₁₄₈, VEGF₁₄₈-Cys, Avi-VEGF₁₆₅, VEGF₁₆₅-Avi, ybbR-VEGF₁₆₅, VEGF₁₆₅-ybbR, FGE-VEGF₁₆₅, VEGF₁₆₅-FGE, Cys-VEGF₁₆₅, VEGF₁₆₅-Cys, Avi-VEGF₁₈₃, VEGF₁₈₃-Avi, ybbR-VEGF₁₈₃, VEGF₁₈₃-ybbR, FGE-VEGF₁₈₃, VEGF₁₈₃-FGE, Cys-VEGF₁₈₃, VEGF₁₈₃-Cys, Avi-VEGF₁₈₉, VEGF₁₈₉-Avi, ybbR-VEGF₁₈₉, VEGF₁₈₉-ybbR, FGE-VEGF₁₈₉, VEGF₁₈₉-FGE, Cys-VEGF₁₈₉, VEGF₁₈₉-Cys, Avi-VEGF₂₀₆, VEGF₂₀₆-Avi, ybbR-VEGF₂₀₆, VEGF₂₀₆-ybbR, FGE-VEGF₂₀₆, VEGF₂₀₆-FGE, Cys-VEGF₂₀₆, and VEGF₂₀₆-Cys.

Fluorophores, isotopes, or other detectable labels are most commonly introduced into a protein through in vitro modifications with suitable amine or thiol reactive reagents. Proteins typically contain multiple amines and, even with the reduced pKa of the amino-terminus of the protein compared to the lysine side chain, this labeling is generally nonspecific. Cysteine labeling is typically more specific than amine labeling as many natural cytosolic proteins lack cysteine residues, and a single cysteine can be added by site-directed mutagenesis without affecting the function of the protein. However, cysteine-based methods alone cannot easily perform multiple modifications to a protein except when the reactivities of the two cysteines are quite different, and not all protein targets allow for the introduction of cysteine without impairing function.

Recombinant DNA construction of protein fusions in which the protein segment has a dedicated site for labeling or could be recognized by a coenzyme ligase fused to a protein of interest could have distinct advantages over conventional protein fusions and chemical or mutagenesis-based site-specific modification of target protein. Advantages of the fusion protein approach over chemical modification and site-specific mutagenesis would be that: (1) the fusion proteins could be specifically labeled by growth of cell cultures in the presence of labeled coenzymes, or be site-specifically labeled under certain mild conditions; (2) if the coenzyme possessed specific binding toward an immobilized ligand, the fusion protein could be readily purified, perhaps in a native form; (3) the protein segment fused to the protein of interest would be much smaller than those commonly used, thus giving less alteration of the chemical and biological properties of the protein of interest; and (4) the tag is usually fused to the N- or C-terminus of the targeted protein, resulting in minimum effect on the protein structure and function. As described above, the tags that can be incorporated onto the N-terminus or C-terminus include: Avi-tag, ybbR-tag, FGE-tag, and Cys-tag.

Chelators

As mentioned above, the VEGF protein can be linked to a PET label via a chelator. The chelator can include, but is not limited to, a macrocyclic chelator, a non-cyclic chelator, and combinations thereof. The macrocyclic chelator can include, but is not limited to, 1,4,7,10-tetraazadodecane-N,N′; N″,N′″-tetraacetic acid (DOTA); 1,4,7-triazacyclononane-1,4,7-triacetic acid (NOTA); 1,4,8,11-tetraazacyclotetradecane-1,4,8,11-tetraacetic acid (TETA); diethylenetriaminepentaacetic (DTPA); CHX-A″-DTPA (where CHX-A is (R)-2-Amino-3-(4-isothiocyanatophenyl)propyl]-trans-(S,S)-cyclohexane-1,2-diamine-pentaacetic acid); CB-TE2A (4,11-bis(carboxymethyl)-1,4,8,11-tetraazabicyclo[6.6.2]hexadecane) and combinations thereof. The number of chelators per VEGF protein can be about 1 to 10. Additional details are described in the Examples.

In an embodiment, the VEGF protein may be labeled through the lysine amines, cysteine thiols, or tyrosine residues. If the label is a radiometal, a cyclic or acyclic chelating agent can be used. In an embodiment where the label is a halogen, it can be directly labeled through tyrosine residues such as by iodination (e.g., ¹²³I-, ¹²⁴I, ¹²⁵I-, and ¹³¹I-). In an embodiment, the antibody can be labeled through lysine or cysteine via prothetic groups or synthons. For example, the isotope labeled in this manner could be ¹⁸F, ⁷⁶Br/⁷⁷Br, or an iodide.

Radioisotope Labels

The radioisotope labels can include, but are not limited to, PET radioisotope. In an embodiment of the present disclosure, the isotope label is a PET radioisotope. The PET radioisotope can include, but is not limited to, ¹⁸F, ⁶⁴Cu, ¹²⁴I, ^{76/77}Br, ⁸⁶Y, ⁸⁹Zr, and ⁶⁸Ga. In an embodiment, the PET isotope is ⁶⁴Cu or ¹⁸F. The number of isotopes per protein can be about 1 to 10.

Methods of Use

Embodiments of this disclosure include, but are not limited to: methods of imaging tissue; methods of imaging precancerous tissue, cancer, and tumors; methods of diagnosing precancerous tissue, cancer, and tumors; methods of monitoring the progress or progression of precancerous tissue, cancer, and tumors; methods of imaging abnormal tissue, and the like. In addition, embodiments of this disclosure include, but are not limited to, methods of imaging tissue that may be affected by ischemic or hypoxic related diseases; methods of diagnosing ischemic or hypoxic related diseases; methods of monitoring the progress of ischemic or hypoxic related diseases; methods of imaging abnormal tissue that may be affected by ischemic or hypoxic related diseases, and the like. Additional details are described the Examples.

Embodiments of the present disclosure can be used to detect (and visualize), quantitate, study, monitor, evaluate, and/or screen, biological events in vivo or in vitro, such as, but not limited to, precancerous tissue, cancer, tumors and related biological events as well as ischemic or hypoxic related diseases.

In general, the labeled VEGF protein (in particular, labeled VEGF₁₂₁ protein) can be used in imaging cancer cells or tissue as well as ischemic or hypoxic related diseases. For example, the labeled VEGF protein is provided to a host in an amount effective to result in uptake of the compound into the cells or tissue of interest. The host is then exposed to an appropriate PET source (e.g., a light source) after a certain amount of time. The cells or tissue that take up the labeled VEGF protein can be detected using a PET imaging system.

In an embodiment, the labeled VEGF protein can be used in imaging cancerous cells, precancerous cells, and tumors. It should be noted that the labeled VEGF proteins are preferentially taken up by the cancerous cells, precancerous cells, and tumors as well as the tissue affected by ischemic or hypoxic related diseases. Thus, the labeled VEGF protein may find use both in diagnosing cancer and in treating cancer as well as ischemic or hypoxic related diseases.

In diagnosing the presence of cancerous cells, precancerous cells, and tumors in a subject, a labeled VEGF protein is administered to the subject in an amount effective to result in uptake of the labeled VEGF protein into the targeted cells. After administration of the labeled VEGF protein, the targeted cells that take up the labeled VEGF protein are detected using PET imaging. Embodiments of the present disclosure can non-invasively image tissue throughout an animal or patient. It should be noted that a similar method can be performed in diagnosing the presence of ischemic or hypoxic related diseases.

The labeled VEGF protein can also be used for patients undergoing chemotherapy/radiotherapy, to aid in visualizing the response of tumor tissue to the treatment. In this embodiment, the cancer tissue is typically visualized and sized prior to treatment, and periodically during chemotherapy/radiotherapy to monitor the tumor size as well as the biological changes that occur long before there is any change in tumor size.

The labeled VEGF protein also finds use as a screening tool in vitro to select compounds for use in treating cancer. The size of an in vitro tumor can be easily monitored in the presence of candidate drugs by incubating the cells with the labeled VEGF protein during or after incubation with one or more candidate drugs.

It should be noted that the amount effective to result in uptake of the compound into the cells or tissue of interest will depend upon a variety of factors, including for example, the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts.

Kits

This disclosure encompasses kits that include, but are not limited to, labeled VEGF proteins (as described in detail above as well as VEGF mutants) and directions (written instructions for their use). The components listed above can be tailored to the particular biological event (e.g., a particular cancer or ischemic or hypoxic related disease) to be monitored as described herein. The kit can further include appropriate buffers and reagents known in the art for administering various combinations of the components listed above to the host cell or host organism.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified. In addition, the phrase “about ‘x’ to ‘y’” includes “about ‘x’ to about ‘y’”.

The above discussion is meant to be illustrative of the principles and various embodiments of the present disclosure. Numerous variations and modifications will become apparent to those skilled in the art once the above disclosure is fully appreciated. It is intended that the following claims be interpreted to embrace all such variations and modifications.

### EXAMPLE

Now having described the embodiments of the disclosure, in general, the example describes some additional embodiments. While embodiments of present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Introduction

For solid tumors and metastatic lesions, tumor vascularity is a critical factor in assessing response to therapy. This disclosure reports the first example of ⁶⁴Cu-labeled VEGF₁₂₁ (VEGF₁₂₁ protein (SEQ ID No: 1)) for positron emission tomography (PET) imaging of vascular endothelial growth factor receptor (VEGFR) expression levels in vivo. VEGF₁₂₁ was labeled with ⁶⁴Cu via a DOTA chelator without significantly affecting the binding affinity to VEGFR2-expressing endothelial cells. MicroPET imaging revealed rapid, specific, and prominent uptake of ⁶⁴Cu-DOTA-VEGF₁₂₁ in small U87MG tumors (high VEGFR2 expression) but significantly lower and sporadic uptake in large U87MG tumors (low VEGFR2 expression). No renal clearance of ⁶⁴Cu-DOTA-VEGF₁₂₁ was observed although the kidney uptake was relatively high likely due to VEGFR1 expression. This study supports clinical translation of ⁶⁴Cu-DOTA-VEGF₁₂₁ to image tumor angiogenesis in patients and to guide anti-angiogenic treatment, especially VEGFR targeted cancer therapy.

Vascular endothelial growth factor (VEGF)-A plays a central role in both normal vascular tissue development and tumor neovascularization. VEGF-A is primarily an endothelial cell-specific angiogenic protein released by a variety of tumor cell lines and found to be expressed in various human tumors. Through alternative splicing of RNA, VEGF may exist as at least seven different molecular isoforms, having 121, 145, 148, 165, 183, 189, or 206 amino acids (VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7)). These isoforms differ not only in their molecular weight but also in their biological properties such as the ability to bind to cell surface heparin sulfate proteoglycans. VEGF₁₂₁ is a soluble, non-heparin-binding variant that exists in solution as a disulfide-linked homodimer containing the full biological and receptor-binding activity of the larger variants.

The angiogenic actions of VEGF are mainly mediated via two closely related endothelium-specific receptor tyrosine kinases, Flt-1 (VEGFR1) and Flk-1/KDR (VEGFR2). Both are largely restricted to vascular endothelial cells and are over-expressed on the endothelium of tumor vasculature; however, they are almost undetectable in the vascular endothelium of adjacent normal tissues. All of the VEGF-A isoforms bind to both VEGFR-1 and VEGFR-2. VEGF and its receptors are over-expressed in a variety of solid tumor biopsy specimens and over-expression of VEGFR2 or VEGF-A has been implicated as poor prognostic markers in various clinical studies. Agents that prevent VEGF-A binding to its receptors, antibodies that directly block VEGFR2, and small molecules that inhibit the kinase activity of VEGFR2, and thereby block growth factor signaling, are all under active development. The contribution of VEGF-A to cancer progression has been highlighted by the recent approval of the humanized anti-VEGF monoclonal antibody bevacizumab (Avastin; Genentech) for first line treatment.

PET has several advantages over SPECT, including 10-fold greater sensitivity, and the increasing implementation of clinical PET and PET/CT scanners can facilitate the translation of novel PET tracers to the clinic.

Successful development of VEGF-based PET imaging could serve as a paradigm for assessment of cancer therapeutics targeting tumor angiogenesis. The ability to non-invasively visualize and quantify tumor VEGFR expression levels could provide new opportunities to document tumor angiogenesis status, more appropriately select patients considered for anti-angiogenic treatment, and monitor anti-angiogenic treatment efficacy. This example reports the first successful example of ⁶⁴Cu (t/_{1/2}, 12.7 h; 39% β⁻; 17.4% β⁺)-labeled VEGF₁₂₁ for PET imaging of tumor angiogenesis and VEGFR expression.

Results

Receptor Binding Assay and Functional Analysis of DOTA-VEGF₁₂₁

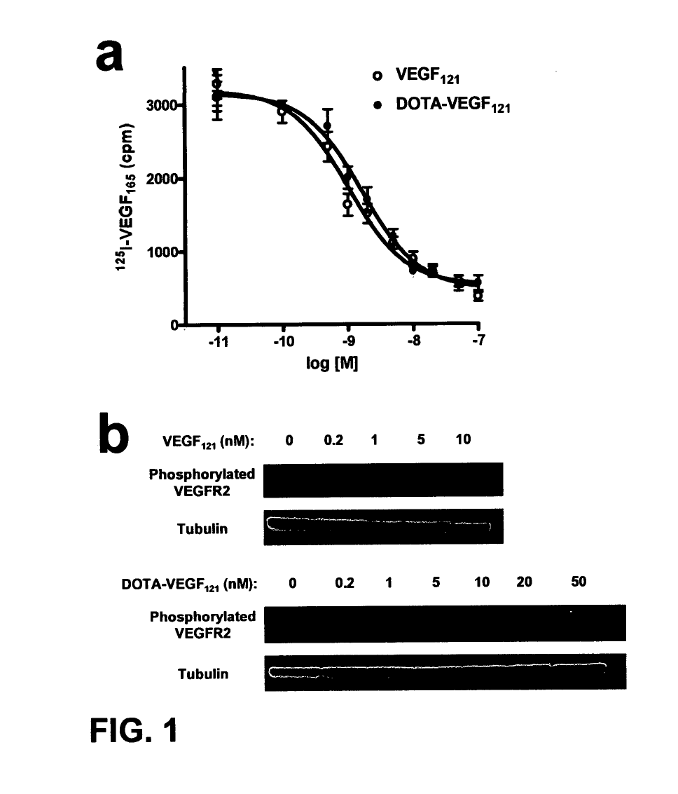

The binding of VEGF₁₂, and DOTA-VEGF₁₂₁ (where DOTA denotes 1,4,7,10-tetraazadodecane-N,N′N″,N″′-tetraacetic acid) to endothelial cells expressing VEGFR2 was assessed using ¹²⁵I-VEGF₁₆₅ as the primary radioligand and VEGF₁₂₁ molecules as competitors. The IC₅₀ values were 1.02 nM and 1.66 nM for VEGF₁₂₁ and DOTA-VEGF₁₂₁, respectively (FIG. 1(a)). The minimal differences in VEGFR2 binding affinity between VEGF₁₂₁ and DOTA-VEGF₁₂₁ suggested that the lysine residues and/or N-terminal amine groups used for DOTA conjugation may not be located at the VEGFR2 binding domain. DOTA conjugation of VEGF₁₂₁ resulted in somewhat decreased functional activity (FIG. 1(b)). However, full functional activity is not required for imaging applications.

Radiolabeling of DOTA-VEGF₁₂₁

⁶⁴Cu-labeling, including the final purification, of DOTA-VEGF₁₂₁ took 90±10 min (n=5), and the radiolabeling yield was 87.39±3.18% (based on 1 mCi ⁶⁴Cu per 10 μg of DOTA-VEGF₁₂₁, n=7). The specific activity of ⁶⁴Cu-DOTA-VEGF₁₂₁ was 87.39±3.18 mCi/mg with radiochemical purity of >98%. The number of DOTA molecules per VEGF₁₂₁ molecule was determined to be 4.33±0.24 (n=3). No significant metabolite peak was observed on radio-HPLC when ⁶⁴Cu-DOTA-VEGF₁₂₁ was incubated with mouse serum at 37° C. for up to 24 h, indicating that the radiotracer is stable in mouse serum.

MicroPET Imaging of ⁶⁴Cu-DOTA-VEGF₁₂₁ in U87MG Tumor-Bearing Mice

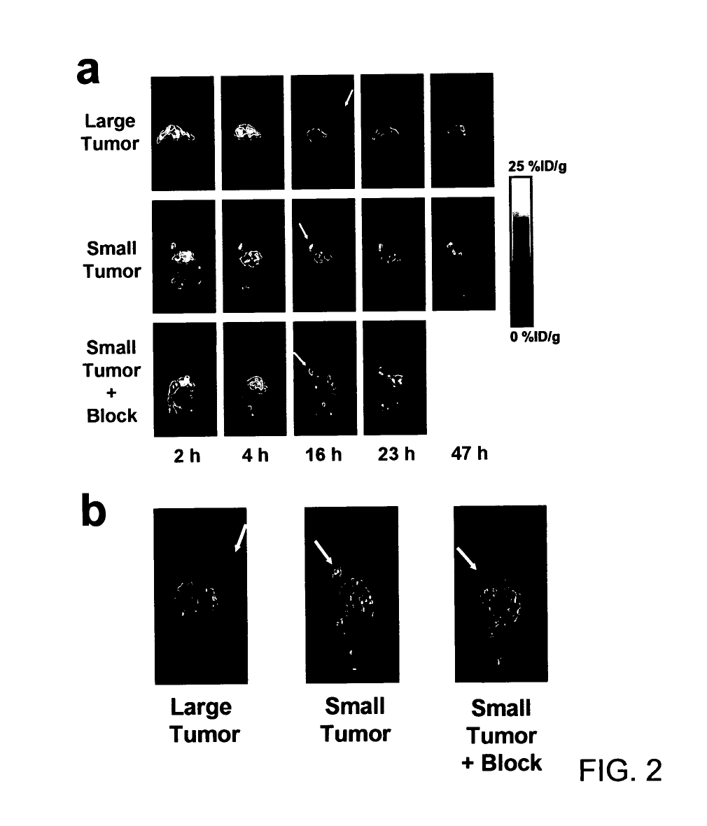

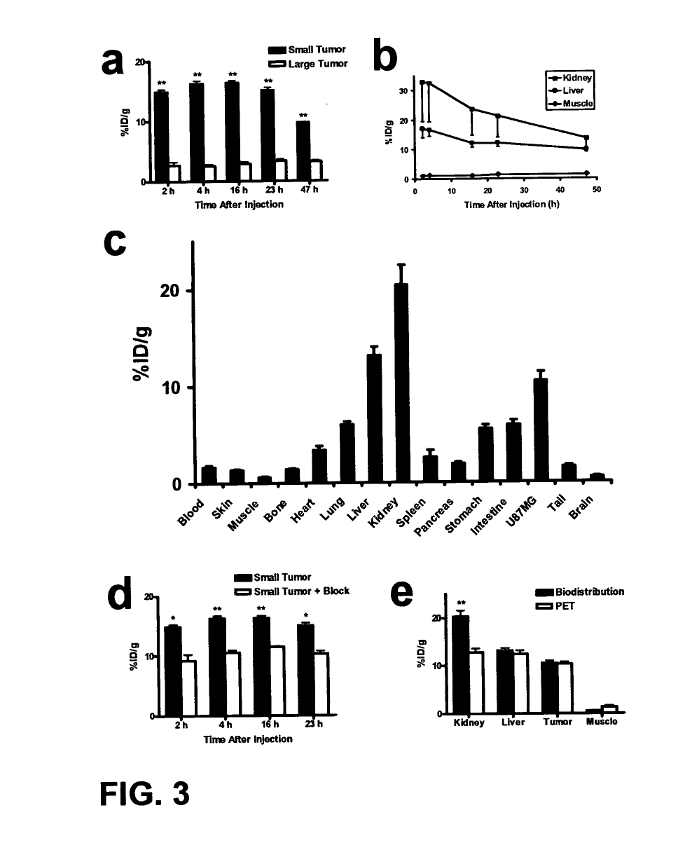

Mice bearing small (tumor volume: 64.9±24.6 mm³, n=3; high VEGFR expression) and large U87MG tumors (tumor volume: 1164.3±179.6 mm³, n=3; low VEGFR expression) were injected via tail vein with approximately 200 μCi of ⁶⁴Cu-DOTA-VEGF₁₂₁ and then subjected to microPET scans at various time points post-injection (p.i.). The coronal slices that contain the tumor are shown in FIG. 2(a). The uptake of ⁶⁴Cu-DOTA-VEGF₁₂₁ into small tumors was rapid and high, reaching 14.89±0.66, 16.26±0.74, 16.33±0.62, 15.06±0.80 percent injected dose per gram of tissue (% ID/g) at 2, 4, 16, and 23 h p.i., respectively. As early as 1 h p.i., the tumor was clearly visible (data not shown due to space limitations). The uptake in the large tumor was at the background level at all time points examined, with the tracer uptake at the peripheral region (˜3-4% ID/g) slightly higher than the necrotic center of the tumor (˜1-2% ID/g). The tumor uptake was significantly different between the small tumors and the large tumors at all time points examined (P<0.01, FIG. 3(a)).

⁶⁴Cu-DOTA-VEGF₁₂₁ exhibited high uptake in the kidneys and liver at early time points (33.03±13.46 and 17.07±3.18% ID/g at 2 h p.i., respectively; n=6, 3 small tumor mice and 3 large tumor mice), and the uptake in most other organs was at background level. The tracer uptake in both the kidneys and the liver dropped steadily over time (FIG. 3(b)). For the kidneys, the tracer uptake was 33.03±13.46, 21.27±7.26, and 13.54±3.50% ID/g at 2, 23, and 47 h p.i., respectively. Although the kidney uptake was high, there was no activity excreted to the urinary bladder, indicating no renal excretion of ⁶⁴Cu-DOTA-VEGF₁₂₁.

Blocking and Biodistribution Studies

To test the VEGFR specificity of ⁶⁴Cu-DOTA-VEGF₁₂₁ in vivo, blocking experiments were carried out where 100 μg VEGF₁₂₁ was injected into small U87MG tumor-bearing mice (tumor volume: 61.30±10.61 mm³, n=3) 30 minutes before administration of ⁶⁴Cu-DOTA-VEGF₁₂₁. As can be seen in both coronal slices (FIG. 2(a)) and two-dimensional whole body projection at 16 h p.i. (FIG. 2(b)), the small U87MG tumor uptake is significantly (P<0.001) lower compared to those mice without VEGF₁₂₁ blocking. After the microPET scans at 23 h p.i., the mice were immediately sacrificed and biodistribution studies were carried out to validate the accuracy of microPET quantification results (FIG. 3(c)). It is of note that although 100 μg of VEGF₁₂₁ (ca. 5 mg/kg) was unable to completely block receptor mediated uptake, the activity accumulation in the tumor was significantly (P<0.01) lower at all time points examined (9.22±1.67, 10.53±0.64, 11.42±0.20, and 10.25±0.84% ID/g at 2, 4, 16, and 23 h p.i., respectively) when compared to the control mice (FIG. 3(d)).

The kidney uptake was also much lower in the mice injected with VEGF₁₂₁ based on microPET scans, however, statistical significance was not achieved likely due to the large variance in kidney uptake between individual animals and/or a potentially insufficient blocking dose of non-radiolabeled VEGF₁₂₁. The variance between each individual animal was not fully understood. When comparing the quantification results obtained from biodistribution studies and PET scans, there is no significant difference between the liver, tumor and muscle (P>0.05, FIG. 3(e)), suggesting that quantification of non-invasive microPET images is able to reflect the biodistribution of radiotracers in these organs. The difference in renal uptake between the two studies was likely due to the heterogeneous tracer uptake in the kidney and the difficulty in region of interest (ROI) analysis because of the irregular shape of kidney.

Immunofluorescence Staining and Western Blot

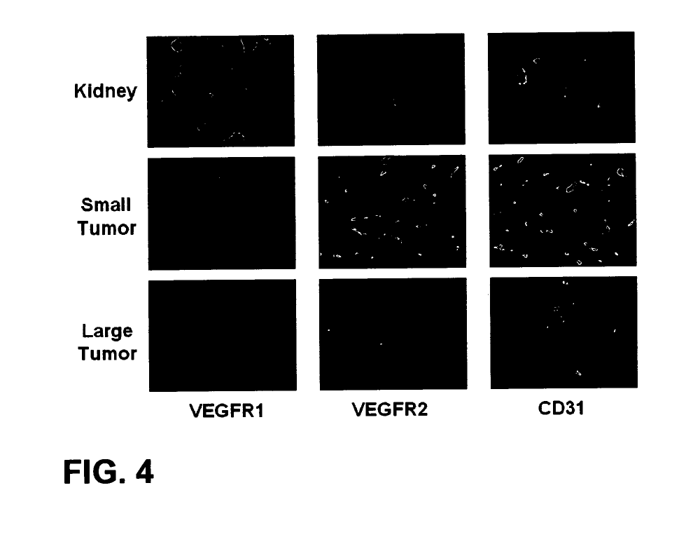

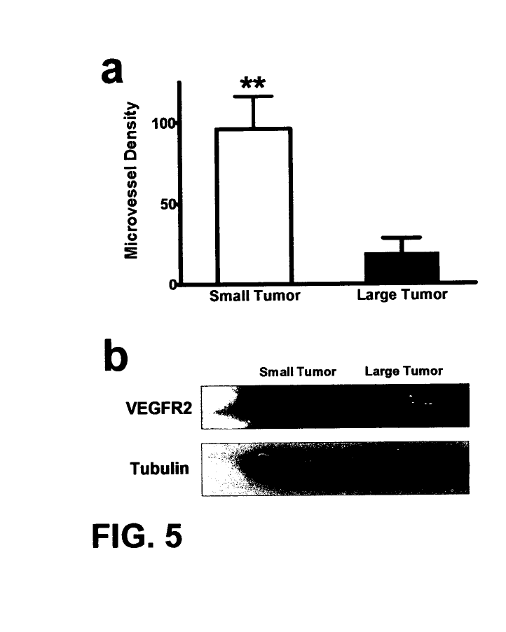

After the decay of most of the radioactivity in the microPET studies, the mice were sacrificed, and the frozen tumor slices were stained for CD31, VEGFR1, and VEGFR2. As can be seen from FIG. 4, VEGFR1 expression was low in both small and large U87MG tumors. VEGFR1 expression is known to be expressed on endothelial cells of pre-glomerular vessels, glomeruli, and post-glomerular vessels in the kidneys, which is most likely responsible for the observed high kidney uptake of ⁶⁴Cu-DOTA-VEGF₁₂₁. Small U87MG tumors had high VEGFR2 expression while the large tumors demonstrated very low VEGFR2 expression, resulting in high tracer uptake in small U87MG tumors but relatively low uptake in the large tumors. There was also a measurable level of VEGFR2 in the kidneys, mostly on the small vessels but not on the large, mature vessels. CD31 staining indicated much higher vascular density in the small U87MG tumor than in the large tumor. The vessels in the small tumor were mostly of regular shape while the vessels in the large U87MG tumor have much larger diameter and the shapes were more irregular. Co-staining of VEGFR2 and CD31 was unsuccessful. However, visual examination of CD31 and VEGFR2 staining of different slices in the same tumor suggests that VEGFR2 and CD31 are co-localized on the newly developed tumor vessels. Microvessel density (MVD) analysis revealed that the small U87MG tumor has significantly higher vessel density (96±19 vessels/mm²) than the large tumor (20±9 vessels/mm²; P<0.01, FIG. 5(a)). Western blot also showed higher VEGFR2 protein level in the small U87MG tumors than in the large tumors (FIG. 5(b)). Due to the low expression level of VEGFR1 in both the small and large U87MG tumors, western blot of VEGFR1 was not obtained. The good correlation of ex vivo results with the in vivo microPET imaging indicates that non-invasive microPET imaging using ⁶⁴Cu-DOTA-VEGF₁₂₁ can reflect the VEGFR expression level in vivo.

Radiation Dosimetry

Human absorbed doses to normal organs from ⁶⁴Cu-DOTA-VEGF₁₂₁ were estimated from microPET imaging quantification data in female Sprague-Dawley rats, assuming that the biodistribution and pharmacokinetics of the tracer in rats and adult human are the same, and the results are presented in Table 1 of Example 1. The tracer uptake of different organs in rats was similar to that of mice. Except for the heart, liver, and kidneys, all other organs exhibited essentially background level of tracer uptake at all time points examined. The tracer was mainly excreted via the hepatic pathway. No activity was observed in the urinary bladder at all time points examined, again indicating that ⁶⁴Cu-DOTA-VEGF₁₂₁, does not undergo renal excretion. The highest radiation-absorbed dose is to the kidneys (1.05±0.27 mGy/MBq). Except for the kidneys and the liver (0.12±0.02 mGy/MBq), all other organs have background level of radiation-absorbed doses. The whole-body absorbed dose was found to be 0.050±0.006 mGy/MBq administered. Although the dose limiting organ is the kidneys, the relatively low radiation dose will unlikely cause any adverse effect as ⁶⁴Cu-DOTA-VEGF₁₂₁ will only be used for imaging application where limited radioactivity (˜3-10 mCi) will be injected in patients.

Discussion

This study demonstrates that ⁶⁴Cu-labeled VEGF₁₂₁ exhibits strong and specific VEGFR binding affinity both in vitro and in vivo. FDA approval of anti-VEGF antibody Avastin and the fact that many other antibody and small molecule inhibitors against VEGFR2 are currently in advanced clinical trials confirms the validity and importance of VEGF/VEGFR2 signaling in anti-cancer therapy. PET imaging using radio-labeled VEGF₁₂₁ is critical in early and sensitive lesion detection, patient selection for clinical trials based on in vivo VEGFR expression quantification, better treatment monitoring and dose optimization based on non-invasive detection of early response to VEGF or VEGFR targeted therapy, as well as elucidating the mechanisms of treatment efficacy underlying VEGF/VEGFR signaling.

It is well accepted that tumor angiogenesis occurs when the tumor reaches a certain size (usually 1-2 mm in diameter), as new blood vessel formation is needed to supply oxygen and nutrients to cancer cells and to remove waste. VEGF-A and its receptors are the best-characterized signaling pathway in developmental angiogenesis as well as tumor angiogenesis. VEGFR2 appears to be the most important receptor in VEGF-induced mitogenesis, angiogenesis, and permeability, while the role of VEGFR1 in endothelial cell function is less clear. During the exponential growth stage, VEGFR expression is highly upregulated on the newly developed tumor vasculature. Being a naturally existing VEGFR ligand, VEGF₁₂₁ offers several advantages over the synthetic small molecule VEGFR ligands or anti-VEGFR antibodies. It has much higher binding affinity to VEGFR (nanomolar range) than reported peptidic VEGFR inhibitors (submicromolar to micromolar range). Compared to antibody-based tracers, VEGF₁₂₁ clears much faster from the blood pool, and the non-targeting organs due to its smaller size (25 kDa for the dimeric form for VEGF₁₂₁). In this study, two different sizes of the same tumor type were selected, hypothesizing that the small tumors have higher VEGFR than the large tumors.

Indeed, in the small U87MG tumor where the diameter is about 4-6 mm, the tumor is at an exponential growth stage. Both immunofluorescence staining (VEGFR1, VEGFR2, and CD31, FIG. 4) and Western blot (FIG. 5b) demonstrated that there is high level of VEGFR2 expression in the tumor. For the large tumor where the tumor diameter is about 10-15 mm, the tumor vessels are mostly mature (much larger diameter when compared to the vessels in the small tumor and the vessel density is also much lower) and both VEGFR1 and VEGFR2 expression are quite low, also confirmed by ex vivo immunofluorescence staining and immunoblot. The ex vivo results support the in vivo PET imaging findings, where the small tumors have much higher ⁶⁴Cu-DOTA-VEGF₁₂₁ uptake (˜16% ID/g) than that in the large tumors (˜2-3% ID/g). PET imaging of other less vascularized tumors (e.g., MDA-MB-435 breast cancer, where the vessel density is quite low when the tumor reaches a certain size) also showed very low ⁶⁴Cu-DOTA-VEGF₁₂₁ uptake (data not shown). Tiny (<2 mm in diameter, where these are very few tumor vessels and therefore very low VEGFR expression) and medium sized U87MG tumors (tumor volume: 200-400 mm³) showed virtually background level and intermediate level (5-8% ID/g) of ⁶⁴Cu-DOTA-VEGF₁₂₁ uptake, respectively (data not shown). These results suggest that the time window of high VEGFR expression is quite narrow. In the clinical setting, the right timing is critical for VEGFR-targeted cancer therapy. PET imaging using ⁶⁴Cu-DOTA-VEGF₁₂₁ can play a very important role in determining whether, and when, to start the VEGFR-targeted cancer therapy as it can provide a straightforward and convenient way to monitor VEGFR expression level in vivo.

In addition to immunofluorescence staining and Western blot analysis, FACS sorting of tumor cells may be another method to quantify VEGFR2 expression. Tumor cells can be harvested and stained for CD31 and/or VEGFR1/VEGFR2. FACS analysis can then be performed to evaluate the VEGFR1/VEGFR2 expression on endothelial cells. Other microvessel markers such as CD105 may also be used to correlate VEGFR expression and MVD in future studies. The VEGFR2 binding affinity of DOTA-VEGF₁₂₁ is comparable to VEGF₁₂₁. Although the functional activity of DOTA-VEGF₁₂₁ is significantly lower than VEGF₁₂₁, this is not a concern for imaging applications. The serial microPET imaging of ⁶⁴Cu-DOTA-VEGF₁₂₁ indicated that ⁶⁴Cu is the optimal isotope for VEGF₁₂₁-based tracer, as the half-life of ⁶⁴Cu (12.7 h) is well suited for the time frame needed to follow the tracer uptake and clearance (48 h in this study, slightly less than 4 half-lives of ⁶⁴Cu).

VEGFR2 specific ligands can also be developed. Such ligands may offer certain advantages such as low renal uptake. Peptidic VEGFR antagonists, which can be labeled with ¹⁸F (more readily available than ⁶⁴Cu), may also be tested and it can allow for high throughput, as usually 1-2 h post-injection is sufficient for a peptide-based tracer to clear from the non-targeted organs and give high contrast PET images. In this study, the VEGFR1 expression level was lower than VEGFR2 expression in both the small and large tumors while VEGFR2 expression is much higher in the small tumors than in the large tumors. Certain levels of VEGFR2 expression in the kidney was also observed. Comparing the CD31 and VEGFR2 staining of the kidney slices, it can be seen that the VEGFR2 expression is mainly localized to the glomerulus but not on the big vessels (e.g., intralobular vein or the efferent arteriole), which is expected since VEGFR2 is mainly expressed on microvessels. Endogenous VEGF isoforms may also compete with ⁶⁴Cu-DOTA-VEGF₁₂₁ in this study. However, the intact VEGFR binding potency of the radiotracer and the fact that the endogenous VEGF concentration is far from saturating the VEGFR resulted in prominent ⁶⁴Cu-DOTA-VEGF₁₂₁ uptake in the high VEGFR-expressing small U87MG tumor.

In summary, this example describes the first successful example of ⁶⁴Cu-labeled VEGF₁₂₁ for PET imaging of tumor VEGFR expression. MicroPET imaging showed rapid and high ⁶⁴Cu-DOTA-VEGF₁₂₁ uptake in small U87MG tumors but background level uptake in the large tumors, corresponding with the VEGFR2 expression level in vivo. Blocking and biodistribution studies confirmed the VEGFR specificity in vivo and validated the quantification results obtained from the non-invasive microPET imaging studies. Immunofluorescence staining and western blot indicated that VEGFR2 is responsible for the high tracer uptake in the small tumor, as VEGFR1 expression is low in both the small and the large U87MG tumors. The success of VEGFR-specific tumor imaging using ⁶⁴Cu-DOTA-VEGF₁₂₁ may be translated into the clinic to characterize the pharmacokinetics, tumor targeting efficacy, dose optimization and dose interval of VEGF or VEGFR targeted cancer therapeutics. It may also significantly aid in patient stratification and treatment monitoring of VEGFR-targeted cancer therapy.

Methods

All commercially available chemical reagents were used without further purification. DOTA was purchased from Macrocyclics, Inc. (Dallas, Tex.). 1-Ethyl-3-[3-(dimethylamino)-propyl]carbodiimide (EDC), N-Hydroxysulfonosuccinimide (SNHS), and Chelex 100 resin (50-100 mesh) were purchased from Aldrich (St. Louis, Mo.). Water and all buffers were passed through Chelex 100 column before use in radiolabeling procedures to ensure that the aqueous buffer is heavy metal free. PD-10 desalting columns were purchased from GE Healthcare (Piscataway, N.J.). Female athymic nude mice were supplied from Harlan (Indianapolis, Ind.) at 4-5 weeks of age. ⁶⁴Cu was obtained from the University of Wisconsin-Madison.

VEGF₁₂₁ Preparation.

The gene for VEGF₁₂₁ was cloned by PCR from endothelial cells (HUVEC), sequenced, and inserted into the pET-32 vector (Novagen). Bacterial host cells were transformed, selected under antibiotic resistance and positive clones were selected for optimal protein expression. Transformed cells were grown at 37° C. with shaking and induced with IPTG at 23° C. Following overnight induction, cells were harvested by centrifugation and then lysed by sonication. The lysate was ultracentrifuged at 40,000 rpm for 90 minutes at 4° C. The supernatant was carefully collected and adjusted to 40 mM Tris-HCl (pH 8.0), 300 mM NaCl, and run through an immobilized metal affinity column. The resin was washed with 40 mM Tris-HCl (pH 8.0), 0.3 M NaCl, 20 mM imidazole buffer and eluted with buffer containing 300 mM imidazole. After pooling fractions containing VEGF₁₂₁, the sample was dialyzed against 20 mM Tris (pH 8.0), 100 mM NaCl and digested with recombinant enterokinase (from Novagen) at room temperature. Enterokinase was removed using agarose-linked soybean trypsin inhibitor. The sample was then dialyzed against 20 mM Tris-HCl (pH 8.0) and purified by elution from to Q Sepharose Fast Flow resin. Purified VEGF₁₂₁ was concentrated and stored in sterile PBS at −20° C.

DOTA Conjugation and Radiolabeling.

A detailed procedure for DOTA conjugation was reported earlier (Wu, Y. et al. MicroPET imaging of glioma αᵥ-integrin expression using ⁶⁴Cu-labeled tetrameric RGD eptide. J. Nucl. Med. 46, 1707-1718 (2005), Cai, W. et al, which is included herein by reference. In vitro and in vivo characterization of ⁶⁴Cu-labeled abegrin, a humanized monoclonal antibody against integrin αᵥβ₃. Cancer Res., in press (2006) which is incorporated herein by reference). DOTA-VEGF₁₂₁ was purified using PD-10 column and concentrated by Centricon filter (Millipore, Bedford, Mass.). The final concentration of DOTA-VEGF₁₂₁ was determined based on UV absorbance at 280 nm using unconjugated VEGF₁₂₁ of known concentrations as standard. ⁶⁴CuCl₂ (2 mCi) was diluted in 300 μL of 0.1 M sodium acetate buffer (NaOAc, pH=6.5), and added to 20 μg of DOTA-VEGF₁₂₁. The reaction mixture was incubated for 1 h at 40° C. with constant shaking. ⁶⁴Cu-DOTA-VEGF₁₂₁ was purified by PD-10 column using PBS as the mobile phase. The radioactive fractions containing ⁶⁴Cu-DOTA-VEGF₁₂₁ was collected and passed through a 0.2 μm syringe filter (Nalge Nunc International, Rochester, N.Y.) for further in vitro and in vivo experiments. ⁶⁴Cu-DOTA-VEGF₁₂₁ was also incubated with complete mouse serum at 37° C. for up to 24 hours to evaluate the stability of this tracer.

The average number of DOTA chelators per VEGF₁₂₁ was determined using a previously reported procedure with slight modifications (Anal. Biochem. 142, 68-78 (1984) which is incorporated herein by reference). Briefly, twenty μg of DOTA-VEGF₁₂₁ in 100 μL 0.1 N NaOAc buffer was added to a defined amount of carrier-added ⁶⁴CuCl₂ solution. The number of DOTA per VEGF₁₂₁ was calculated using the following equation:
number of DOTA per VEGF₁₂₁=moles(Cu²⁺)×yield/moles(DOTA-VEGF₁₂₁)
The results were expressed as mean ±SD (n=3).
Cell Lines and Animal Model.

The U87MG human glioblastoma cell line was obtained from American Type Culture Collection (ATCC, Manassas, Va.) and cultured under standard condition. Porcine aortic endothelial cells that express human KDR (PAE/KDR) were cultured in Ham's F-12 medium containing 10% fetal calf serum (Sigma). Animal procedures were performed according to a protocol approved by Stanford University Institutional Animal Care and Use Committee (IACUC). The U87MG tumor model was generated by subcutaneous injection of 5×10⁶ cells in 50 μL PBS into the front leg of the mice. The mice were subjected to microPET imaging studies when the tumor volume reached about 60 mm³ (small tumor, 7-10 days after inoculation) or 1200 mm³ (large tumor, 4 weeks after inoculation).

Cell Binding Assay and Functional Assay.

A detailed procedure of the cell binding assay has been reported earlier (J. Nucl. Med. 47, 113-121 (2006), J. Med. Chem. 48, 1098-1106 (2005) which are incorporated herein by reference). Receptor binding affinity of VEGF₁₂, and DOTA-VEGF₁₂₁ was analyzed by a PAE/KDR cell binding assay using ¹²⁵I-VEGF₁₆₅ as the radioligand. To determine the functional activity of DOTA-VEGF₁₂₁, PAE/KDR cells were stimulated by serial concentrations of VEGF₁₂₁ or DOTA-VEGF₁₂₁ for 5 minutes, and the cell lysate were immunoblotted by anti-phosphorylated VEGFR2 antibody (Abcam Inc, Cambridge, Mass.).

MicroPET Studies.

PET imaging of tumor-bearing mice was performed on a microPET R4 rodent model scanner (Concorde Microsystems, Knoxville, Tenn.) as described earlier (Xiong, Z. et al. Imaging chemically modified adenovirus for targeting tumors expressing integrin αᵥβ₃ in living mice with mutant herpes simplex virus type 1 thymidine kinase PET reporter gene. J. Nucl. Med. 47, 130-139 (2006), Chen, X. et al, which is include herein by reference. Integrin αᵥβ₃-targeted imaging of lung cancer. Neoplasia 7, 271-279 (2005) which is incorporated herein by reference). For each microPET scan, three-dimensional ROIs were drawn over the tumor, liver, kidneys, and muscle on decay-corrected whole-body coronal images. The average radioactivity concentration (accumulation) within a tumor or an organ was obtained from mean pixel values within the ROI volume, which were converted to counts per milliliter per minute by using a conversion factor. Assuming a tissue density of 1 g/mL, the counts per milliliter per minute were converted to counts per gram per minute, and then divided by the injected dose (ID) to obtain an imaging ROI-derived % ID/g. Mice bearing small U87MG tumors were also imaged using ⁶⁴Cu-DOTA-VEGF₁₂₁ injected 30 minutes after injection of 100 μg of VEGF₁₂₁ (n=3). A total of nine mice were used for this study: three small tumor, three large tumor, and three mice for blocking and biodistribution studies.

Radiation Dosimetry Extrapolation to Human.

Estimated human dosimetry was calculated from microPET imaging results on Sprague-Dawley female rats (Harlan) injected with about 1 mCi of ⁶⁴Cu-DOTA-VEGF₁₂₁ (n=3), assuming that the biodistribution of the tracer in rats in the same as in adult human. The rats were scanned at two bed positions in order to cover the whole body, and ROI analysis was carried out on major organs. Time-activity curves were generated from the mean values obtained in rats for each organ of interest. Source organ residence times for the human model were calculated by integrating a mono-exponential fit to the experimental biodistribution data for major organs (heart, lung, liver, kidneys, and spleen) and the whole body. The source organ residence times obtained forthwith were used with a standard quantitation platform Organ Level Internal Dose Assessment (OLINDA; Vanderbilt University).

Western Blot.

U87MG tumor tissue protein was extracted using T-PER tissue protein extraction buffer, and the concentration was determined using microBCA protein assay kit (Pierce Biotechnology, Inc., Rockford, Ill.). After SDS-PAGE separation of 100 μg of total protein, it was transferred to a polyvinylidene fluoride (PVDF) membrane (Invitrogen Corp., Carlsbad, Calif.) and incubated at room temperature with 5% non-fat milk blocking buffer. The blots were then incubated overnight at 4° C. with rabbit anti-VEGFR2 primary antibody (Lab Vision Corp., Fremont, Calif.) followed by incubation at room temperature for 1 hour with HRP conjugated anti-rabbit antibody (GE Healthcare, Piscataway, N.J.). The bands were detected using ECL western blotting detection system (GE healthcare). Tubulin was used as loading control.

Immunofluorescence Staining.

U87MG tumor and kidney frozen tissue slices (5 μm thick) were fixed with ice cold acetone for 10 minutes and dried in air for 30 minutes. The slices were rinsed with PBS for 2 minutes and blocked with 10% donkey serum for 30 minutes at room temperature. The slices were incubated with rat anti-mouse VEGFR2 antibody overnight at 4° C. and visualized using Cy3-conjugated donkey anti-rat secondary antibody (1:200, Jackson ImmunoResearch Laboratories, Inc., West Grove, Pa.). For VEGFR1 staining, the tissue slices were incubated with rabbit anti-mouse VEGFR1 antibody (1:50, Lab Vision Corp.) at room temperature for 1 hour and visualized with Cy3-conjugated donkey anti-rabbit secondary antibody (1:200, Jackson ImmunoResearch Laboratories, Inc.). For CD31 staining, the slices were incubated with rat anti-mouse CD31 antibody (1:100, BD Biosciences, San Jose, Calif.) at room temperature for 1 hour and visualized with Cy3-conjugated donkey anti-rat secondary antibody (1:200, Jackson ImmunoResearch Laboratories, Inc.).

After CD31 staining, 7 random views in both the center and the periphery of the tumor slices were selected for MVD analysis using an observer-set threshold to distinguish vascular elements from surrounding tissue parenchyma. The vessel that contains branching points was counted as a single vessel. The number of vessels counted was divided by the field of view to yield MVD as vessels/mm².

Statistical Analysis

Quantitative data were expressed as mean ±SD. Means were compared using One-way ANOVA and student's t-test. P values<0.05 were considered statistically significant.

TABLE 1
Example 1. Estimated radiation absorbed doses to an adult human after
intravenous injection of ⁶⁴Cu-DOTA-VEGF₁₂₁ based on the microPET
imaging data obtained in female Sprague-Dawley rats (n = 3).

| Organ | mGy/MBq(SD) | rad/mCi (SD) |
|---|---|---|
| Adrenals | 3.62E−02 (1.87E−03) | 1.34E−01 (6.56E−03) |
| Brain | 1.61E−02 (1.13E−03) | 5.96E−02 (4.08E−03) |
| Breasts | 1.68E−02 (9.64E−04) | 6.22E−02 (3.76E−03) |
| Gallbladder Wall | 3.43E−02 (8.02E−04) | 1.27E−01 (3.00E−03) |
| LLI Wall | 2.02E−02 (1.04E−03) | 7.49E−02 (3.93E−03) |
| Stomach | 2.48E−02 (3.06E−04) | 8.95E−02 (1.79E−03) |
| ULI Wall | 2.45E−02 (5.20E−04) | 9.06E−02 (1.82E−03) |
| Heart | 2.20E−02 (1.07E−03) | 8.14E−02 (3.93E−03) |
| Kidneys | 1.05E+00 (2.72E−01) | 3.87E+00 (1.01E+00) |
| Liver | 1.17E−01 (1.88E−02) | 4.33E−01 (6.89E−02) |
| Lungs | 2.03E−02 (1.03E−03) | 7.51E−02 (3.73E−03) |
| Muscle | 1.96E−02 (7.51E−04) | 7.28E−02 (2.77E−03) |
| Ovaries | 2.12E−02 (9.81E−04) | 7.84E−02 (3.72E−03) |
| Pancreas | 3.26E−02 (7.09E−04) | 1.21E−01 (2.52E−03) |
| Skin | 1.63E−02 (8.39E−04) | 6.05E−02 (3.09E−03) |
| Spleen | 8.45E−02 (9.79E−03) | 3.13E−01 (3.63E−02) |
| Testes | 1.72E−02 (1.16E−03) | 6.35E−02 (4.19E−03) |
| Thymus | 1.86E−02 (1.14E−03) | 6.87E−02 (4.19E−03) |
| Thyroid | 1.77E−02 (1.18E−03) | 6.56E−02 (4.37E−03) |
| Urinary | 1.95E−02 (1.18E−03) | 7.22E−02 (4.39E−03) |
| Uterus | 2.11E−02 (1.04E−03) | 7.82E−02 (3.81E−03) |
| Effective Dose | 5.03E−02 (5.50E−03) | 1.86E−01 (2.07E−02) |

* LLI = lower large intestine; ULI: upper large intestine

### Example 2

Coronary artery disease (CAD) is a major cause of morbidity and mortality in the Western world and thus elucidation of the pathological mechanisms involved in the development and progression of CAD are of critical importance and have the potential to significantly impact the development and assessment of different therapeutic strategies. Myocardial infarction (MI), one of the most significant consequences of CAD, leads to an up-regulation of inflammatory cytokines, such as interleukin-6, collagenases (e.g. matrix metalloproteinases-MMPs-), and different growth factors what can lead to tissue remodeling. Vascular endothelial growth factor (VEGF) is the most prominent member of a family of growth factors that has been strongly associated with angiogenic stimuli in different pathophysiological situations, and likely plays a role in left ventricular (LV) remodeling after MI. Previous studies have shown that MI leads to an initial increase in the expression of VEGF, and subsequent increase in the expression of the VEGF receptors.

Currently, most of the information we have regarding biological pathways involved in myocardial LV remodeling after myocardial infarction derives from ex-vivo tissue analysis. Study of biological pathways in the living subject would permit a more physiological assessment of these pathways as well as longitudinal monitoring of changes that may occur. However until recently, it was not possible to assess biological pathways in the living subject. Advances in the development of molecular imaging probes have allowed scientists to start addressing these questions. Recently, efforts have been made to develop new probes to image and study biological pathways in the living subject after MI. Using molecular imaging strategies, investigators have shown that after MI there is an up-regulation of MMP's, in particular MMP-2 and MMP-9. Furthermore, significant efforts have been made to image some of the effects of LV remodeling after MI, such as myocardial neovascularization. Specifically, Meoli et. al. have demonstrated an increased expression of αvβ3 integrin (a transmembrane protein that is only expressed in newly formed vessels) after MI¹⁸. However, the biological pathways that underlie the remodeling response after MI remain to be determined. To better define the factors that may be involved in the neovascularization response after MI, our laboratory has recently developed a positron emission tomography (PET) tracer (probe) that binds to VEGF receptors in the living animal (J Nucl Med. 2006; 47:2048-2056, which is include herein by reference). We have recently shown, using small animal tumor models, the feasibility of assessing the presence of VEGF receptor in normal and disease states. The use of non-invasive imaging modalities will permit us, for the first time, to monitor receptor expression in the intact subject.

Thus, we hypothesize that myocardial infarction will be associated with an increase in the expression of VEGF receptor and that such expression can be serially monitored using PET.

Materials and Methods

Protocol Design

Protocols were approved by the Stanford Animal Research Committee and conformed with the Guide for the Care and Use of Laboratory Animals published by the U.S. National Institutes of Health (NIH Publication No. 85-23, revised 1996, which is include herein by reference). Animals were divided into two groups: sham operated (n=3) and myocardial infarction (n=8). On day -4, animals underwent high resolution ultrasound for assessment of baseline cardiac function. On day 0, coronary artery ligation was induced in MI animals, while control animals where sham operated. Three days after surgery, cardiac function was re-evaluated to confirm the presence and assess the extent of MI. Micro-PET imaging was performed on days -4, 3, 10, 17, and 24 after the induction of myocardial infarction. On day 24, animals were euthanized and tissue was harvested for ex-vivo studies.

Induction of Myocardial Infarction

Induction of MI was done as previously described by our laboratory (Circulation. 2004; 110:685-91, which is include herein by reference). Adult female Sprague-Dawley rats (weight 150-200 g; Charles River Laboratories, Wilmington, Mass.) were used for this study. On the day of surgery, anesthesia was induced with isoflurane (5%) and the animals were intubated for mechanical ventilation. The anesthesia was then maintained with isoflurane (2%). MI was induced by ligation of the left anterior descending coronary artery 2 to 3 mm from the tip of the left auricle with a 7-0 polypropylene suture. This resulted in myocardial blanching and ST-segment elevation on an ECG monitor (Silogic EC-60 model, Silogic, Stewartstown, Pa.). In the sham operated animals, a suture was placed in the myocardium (without ligating the left coronary artery).

Assessment of Left Ventricular Contractility with Echocardiography

Cardiac function was assessed as previously described (Circulation. 2004; 110:685-91, which is include herein by reference). Briefly, rats received isofluorane (2%) for general anesthesia and were placed on the scanning table. Echocardiographic images were obtained using a dedicated small animal high-resolution-imaging unit and a 30-MHz linear transducer (Vevo 770®, Visualsonics, Toronto, Canada). Using the parasternal short axis view, left ventricular end-diastolic and end-systolic diameters (LVEDD and LVESD, respectively) were measured, and left ventricular fractional shortening was calculated as =(LVEDD-LVESD)/LVEDD*100, as described previously. All measurements were averaged on 3 consecutive cardiac cycles.

PET Probe Synthesis

Radiosynthesis of ⁶⁴Cu-DOTA-VEGF₁₂₁. All commercially available chemical reagents were used without further purification. DOTA (1,4,7,10-tetraazadodecane-N,N′,N″,N′″-tetraacetic acid) was purchased from Macrocyclics, Inc. (Dallas, Tex.) and Chelex 100 resin (50-100 mesh) was purchased from Aldrich (St. Louis, Mo.). PD-10 columns were purchased from GE Healthcare (Piscataway, N.J.). ⁶⁴Cu was obtained from University of Wisconsin-Madison.

Detailed procedure for the synthesis of ⁶⁴Cu-DOTA-VEGF₁₂₁ (VEGF₁₂₁ protein (SEQ ID No: 1)) has been reported earlier (J Nucl Med. 2006; 47:2048-2056, which is include herein by reference). DOTA-VEGF₁₂₁ was purified using a PD-10 column and concentrated by Centricon filter units (Ultracel YM-10, Millipore, Bedford, Mass.). ⁶⁴Cu-labeling was carried out in 0.1 M sodium acetate buffer (NaOAc, pH=6.5) at 40° C. using 10 μg of DOTA-VEGF₁₂₁ per mCi of ⁶⁴Cu. The radiolabeling yield of ⁶⁴Cu-DOTA-VEGF₁₂₁ was 87.4±3.2%, with a specific activity of 3.2±0.1 GBq/mg and a radiochemical purity of >98%. To determine the specificity of this probe for the VEGF receptor we performed cell binding assays. The detailed procedure for the cell-binding assay has been reported earlier (J Nucl Med. 2006; 47:2048-2056, which is include herein by reference). Receptor-binding affinity of VEGF₁₂₁ and DOTA-VEGF₁₂₁ was analyzed by PAE/KDR cell-binding assay using ¹²⁵I-VEGF₁₆₅ as the radioligand. To determine the serum stability, ⁶⁴Cu-DOTA-VEGF₁₂₁ was incubated with complete rat serum at 37° C. for up to 4 h. At different time points, aliquots of the mixture were injected onto an analytical HPLC system (Vydac protein C4 column 214TP54; 5 μm, 250±4.6 mm; flow rate: 1 mL/min). The radioactive peaks of ⁶⁴Cu and ⁶⁴Cu-DOTA-VEGF₁₂₁ were each integrated to calculate the percentage of intact tracer.

Radiosynthesis of ¹⁸F-FDG. ¹⁸F-FDG synthesis was performed at the Stanford Cyclotron Unit, as previously described (J Label Compd Radiopharm. 2002; 45:435-447, which is include herein by reference).

MicroPET Scanning.

MicroPET scanning was done using previously described methods (Circulation. 2004; 110:685-91, which is include herein by reference). Animals were anesthetized with isofluorane (2%) and injected with approximately 1 mCi (37 MBq) of ⁶⁴Cu-DOTA-VEGF₁₂₁ via the tail vein and allowed to recover. To determine the best signal/background ratio, animals were scanned at 1, 4, 18 and 24 hours after injection of the tracer. At the time of scanning, animals were anesthesized with isofluorane (2%) and prone positioned on the microPET Concorde R4 rodent model scanning gantry (Siemens AG, Malvern, Pa.). The scanner has a computer-controlled bed and 10.8-cm transaxial and 8-cm axial fields of view (FOV). Pixel size was of 0.845×0.845×1.2 mm, and a slice thickness of 0.845 mm and full width half maximum of 1.66, 1.65 and 1.84 mm for tangential, radial, and axial orientation, respectively. It has no septa and operates exclusively in the 3-dimensional list mode. A 15 minute static acquisition was performed with the mid thorax in the center of the field of view, and images reconstructed using a filtered back projection algorithm. Uptake was calculated as % ID/gram of tissue using the AMIDE software. In each scan, three different regions of interest (ROIs), 2 mm in diameter, were drawn over the myocardial uptake within the infarcted area, and the mean % ID/g of tissue was averaged. No correction was performed for partial volume effects. In addition, signal from the infarcted area was compared to the contralateral myocardium (at the septal level), which was taken as background signal, and expressed as signal/background ratio.

To confirm the myocardial origin of the ⁶⁴Cu-DOTA-VEGF₁₂₁ detected signal, we performed viability studies using ¹⁸F-2-fluoro-2-deoxy-glucose (¹⁸F-FDG). Immediately after the ⁶⁴Cu-DOTA-VEGF₁₂₁ scan, animals were kept in the scanning gantry (for image co-registration), and injected with 500 μCi (18.5 MBq) of ¹⁸F-FDG (Stanford Cyclotron Unit, Stanford, Calif.). One hour after FDG injection, a 15 min static acquisition was performed with the mid thorax in the center of the FOV, and images reconstructed using a 2D OSEM algorithm. Uptake was calculated as % ID/gram of tissue.

MicroCT Scanning

To anatomically localize the tracer signal obtained using microPET, animals from both groups were also scanned in a microCT scanner (eXplore RS MicroCT System, GE Healthcare, Piscataway, N.J.). Immediately after PET scanning, and using fiducial markers for co-registration, animals were transported to the microCT scanning gantry, positioned and scanned at a voxel resolution of 97 microns (scanning time of 7 minutes). Images were reconstructed using GE built-in software (Microview, GE Healthcare, Piscataway, N.J.). CT and PET datasets were loaded into AMIDE, and fiducial markers co-registered for alignment of datasets.

Ex Vivo Validation of VEGF Receptor Expression

Autoradiography

To further confirm that the signal obtained in microPET was of myocardial origin, immediately after scanning animals from each group (n=3 in each group) were euthanized and the heart harvested. Heart samples were frozen on dry ice and 30 μm slices were obtained (Bright 5030/WD/MR cryomicrotome (Hacker Instruments, Fairfield, N.J.), and exposed for 6-7 hours on a Phosphoimager plate (Perkin Elmer, Wellesley, Mass.), developed in a Cyclone (Perkin Elmer, Wellesley, Mass.), and read using Optiquant software (Packard Instrument Co., Meriden, Conn.)¹⁹.

To determine the specificity of the ⁶⁴Cu-DOTA-VEGF₁₂₁ for the VEGF receptor we performed a separate in-vitro receptor autoradiography blocking study using ¹²⁵VEGF₁₂₁ as radioligand. Briefly, 20 μm slides, from MI and sham operated animals at day 3 post operatively, were fixed with cold acetone for 10 min and dried in the air for 30 min. ¹²⁵I-VEGF₁₆₅ (2,200 Ci/mmol; GE Healthcare) was used to measure the VEGF receptor level. Sections were preincubated twice for 15 min each in PBS buffer. Incubation (2 h) was done in the same buffer plus 0.1% BSA and 50 pM of ¹²⁵I-VEGF₁₆₅ (VEGF₁₆₅ protein (SEQ ID No: 4)). The sections were washed four times for 15 min each at 4° C. in preincubation buffer, dipped in ice-cold water, air-dried, and placed against Super Resolution screen (Packard, Meriden, Conn.) and exposed overnight. Screens were then laser-scanned with the Packard Cyclone. VEGF₁₂₁ (1 μM) was used to define nonspecific binding¹⁹.

VEGF Receptor Immunostaining.

Myocardial frozen tissue slices (5 μm in thickness) were fixed with ice cold acetone for 10 minutes and dried in air for 30 minutes. The slices were rinsed with PBS for 2 minutes and blocked with 10% donkey serum for 30 minutes at room temperature. The slices were incubated with rabbit anti-rat VEGFR2 antibody overnight at 4° C. and visualized using Cy3-conjugated donkey anti-rat secondary antibody (1:200, Jackson ImmunoResearch Laboratories, Inc., West Grove, Pa.). For VEGFR1 staining, the tissue slices were incubated with rabbit anti-rat VEGFR1 antibody (1:50, Lab Vision Corp., Fremont, Calif.) at room temperature for 1 hour and visualized with Cy3-conjugated donkey anti-rabbit secondary antibody (1:200, Jackson ImmunoResearch Laboratories, Inc., West Grove, Pa.). Immunofluorescence was visualized in an Axiovert 200M microscope (Carl Zeiss, Germany) using a Ds-red filter (excitation: 545 nm, emission: 620 nm).

Statistical Analysis

Data are expressed as mean ±SEM. Statistical analysis was used using the unpaired Student t-test with unequal variance (for comparison between sham operated and MI animals) and paired Student t-test for comparison before and after MI. To exclude artifacts related to greater wall motion of the contralateral wall, comparison were always done on the anterolateral wall. A p value of <0.05 was considered statistically significant.

Results

General Characteristics/Assessment of Cardiac Function

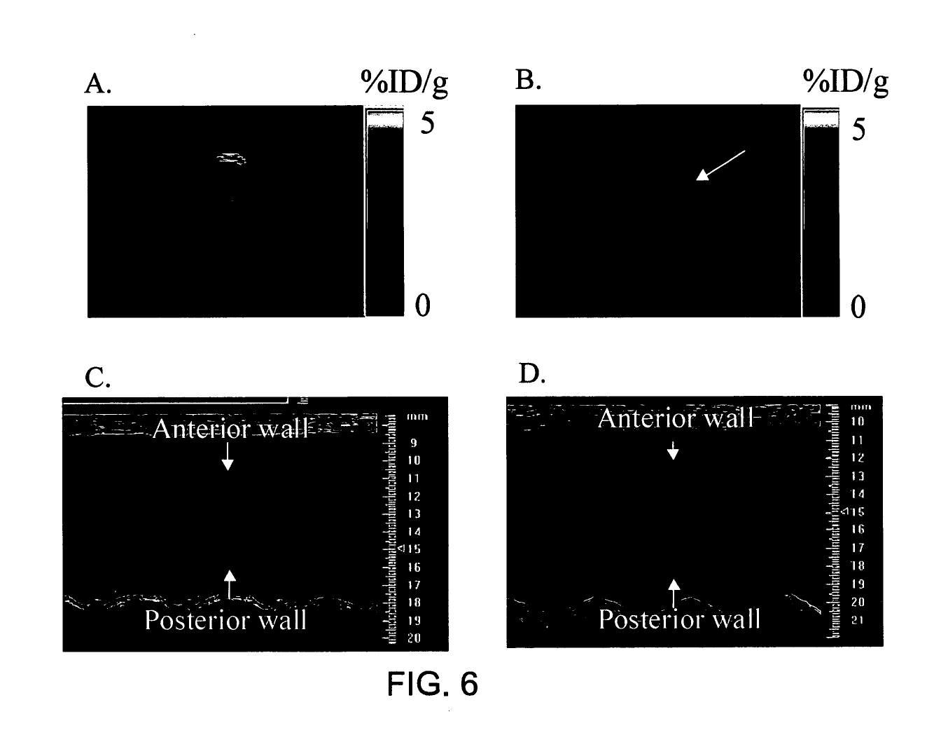

There was no difference in weight or heart rate between sham (n=3) and MI (n=8) animals. At baseline (day: -4), animals from both groups had similar myocardial cardiac function, as assessed by fractional shortening (Table 1). In the experimental group MI induction led to a defect in ¹⁸F-FDG uptake, as assessed by PET (FIG. 6B) and akinesis of the anterolateral wall with a significant decrease in cardiac function (assessed by M-mode high resolution ultrasound at day 10 post-operatively, FIG. 1D and Table 1). On the other side, in sham-operated animals, there was no uptake defect in ¹⁸F-FDG PET (FIG. 6A) and cardiac function remained stable (FIG. 6C and Table 1, Example 2). The MI procedure had a mortality of 15%, similar to that previously reported by other investigators.

Table 1, Example 2. General group characteristics at both baseline and after either sham operation or myocardial infarction. There was no difference in weight, heart rate, or baseline parameters of cardiac function. After MI, there was a decrease in cardiac function, as evidenced by a decrease in fractional shortening, while in sham animals cardiac function was relatively preserved.

TABLE 1
Example 2

| | |
|---|---|
| Sham | MI |

General characteristics

| | | |
|---|---|---|
| Weight (grams) | 184 ± 7 | 182 ± 9 |
| Heart rate (bpm) | 323 ± 20 | 330 ± 15 |
| Cardiac function | | |
| parameters | | |

Baseline

| | | |
|---|---|---|
| LVEDD (mm) | 593 ± 39 | 639 ± 31 |
| LVEDSD (mm) | 264 ± 5 | 314 ± 24 |
| FS (%) | 55.5 ± 3.6 | 50.7 ± 5.4 |

Post-operatively

| | | |
|---|---|---|
| LVEDD (mm) | 623 ± 24 | 665 ± 41 |
| LVEDSD (mm) | 332 ± 23 | 568 ± 45 |
| FS (%) | 46.7 ± 3.4 | 20.7 ± 3.4* |

*p < 0.05 compared to Sham. MI: myocardial infarction, LVEDD: left ventricular end diastolic diameter, LVESD: left ventricular end systolic diameter, FS: Fractional shortening.

Radiolabeling of ⁶⁴Cu-DOTA-VEGF₁₂₁

⁶⁴Cu-DOTA-VEGF₁₂₁ was stable in rat serum, having at least 75% stability one hour after incubation in rat serum and remaining stable for 4 hours, with 3-5% free ⁶⁴Cu and the rest partial compound and metabolites. The binding of VEGF₁₂₁ and DOTA-VEGF₁₂₁ to endothelial cells expressing VEGFR2 was assessed using ¹²⁵I-VEGF₁₆₅ as the radioligand. The 50% inhibitory concentration (IC₅₀) values were 1.11 nmol/L and 1.59 nmol/L for VEGF₁₂₁ and DOTA-VEGF₁₂₁, respectively.

Assessment of Myocardial VEGF Receptor Expression in Living Rats

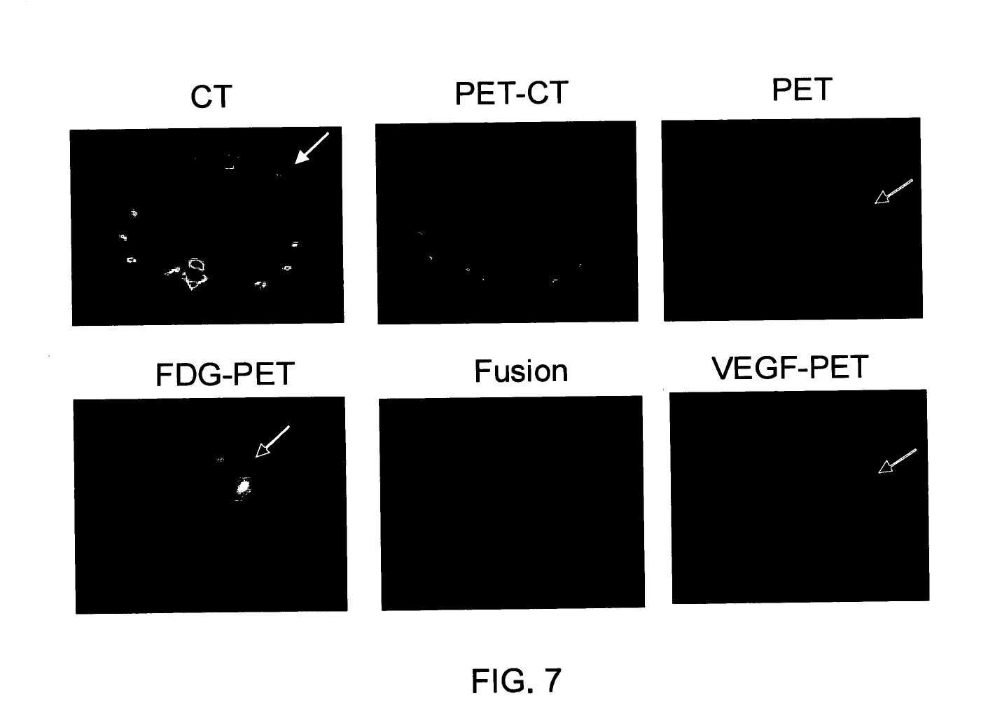

To localize the anatomical region of the ⁶⁴Cu-DOTA-VEGF₁₂₁ microPET detected signal, we performed microCT on MI animals. The top left panel of FIG. 7 shows the microCT image, anatomically separating the myocardium from the chest wall, while the top right image shows a representative image of the ⁶⁴Cu-DOTA-VEGF₁₂₁ PET image from the same animal (day 3 after MI). In the top center panel, the fused image clearly demonstrates the myocardial origin of the ⁶⁴Cu-DOTA-VEGF₁₂₁ signal.

As mentioned above, in MI animals ¹⁸F-FDG (for assessment of myocardial viability, FIG. 7, bottom left) was injected immediately after the ⁶⁴Cu-DOTA-VEGF₁₂₁ scan (FIG. 7, bottom right). ¹⁸F-FDG and ⁶⁴Cu-DOTA-VEGF₁₂₁ images were fused (FIG. 7, center) showing that the ⁶⁴Cu-DOTA-VEGF₁₂₁ myocardial signal matched extremely well to areas of infarcted myocardium as evidenced by a lack of ¹⁸F-FDG uptake. On the other hand, in sham-operated animals, there were no infarcted areas, and thus no lack of ¹⁸F-FDG uptake (FIG. 6A). Furthermore, post operatively, animals (both sham and MI groups) had increased uptake of ¹⁸F-FDG at the level of the surgical wound (FIGS. 6A and 6B, and FIG. 7, green arrows), consistent with an inflammatory response.

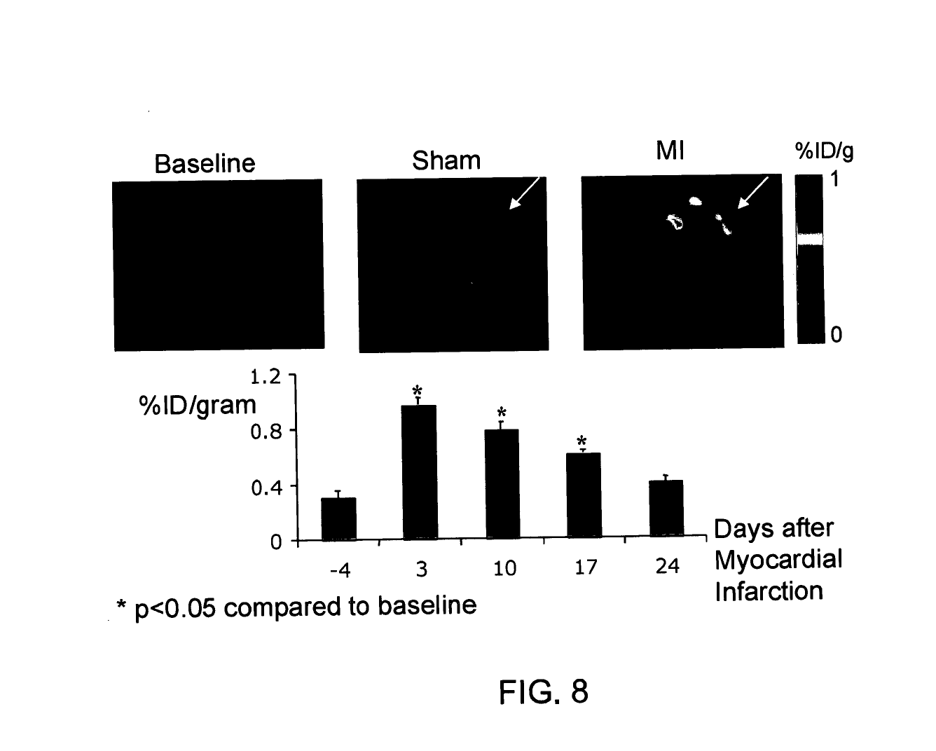

The best ⁶⁴Cu-DOTA-VEGF₁₂₁ signal/background ratio was obtained one hour after injection of the tracer and was thus used for signal quantification and analysis in this study (1 h: 2.60±0.30, 4 h: 2.43±0.46, no consistent signal could be detected at the 18 and 24 hours scans). At day -4 (before MI induction or sham operation), there was no significant myocardial uptake of ⁶⁴Cu-DOTA-VEGF₁₂₁ (0.30±0.07% ID/g, FIG. 8, top left). At day 3 postoperatively, sham operated animals did not have significant myocardial uptake (0.31±0.06% ID/g, FIG. 8, top middle). MI induction was associated with a significant increase in uptake of ⁶⁴Cu-DOTA-VEGF₁₂₁ in the anterolateral wall of the myocardium (MI: 1.04±0.09% ID/g, p<0.05 compared to baseline, FIG. 8, top right). Similarly to ¹⁸F-FDG, animals from both groups had increased uptake of ⁶⁴Cu-DOTA-VEGF₁₂, at the level of the surgical wound (FIG. 8, yellow arrow), probably reflecting an angiogenic response during the wound healing process (sham: 0.99% ID/g, MI: 1.03% ID/g, p=0.39).

The microPET ⁶⁴Cu-DOTA-VEGF₁₂₁ myocardial uptake was higher at day 3 post-operatively, and decreased over time until it reached baseline levels at day 24 (FIG. 8, bottom). Importantly, the tracer uptake was only seen in the areas supplied by the ligated coronary artery, and not in remote areas.

Ex-Vivo Assessment of VEGF Receptor Expression

Autoradiography

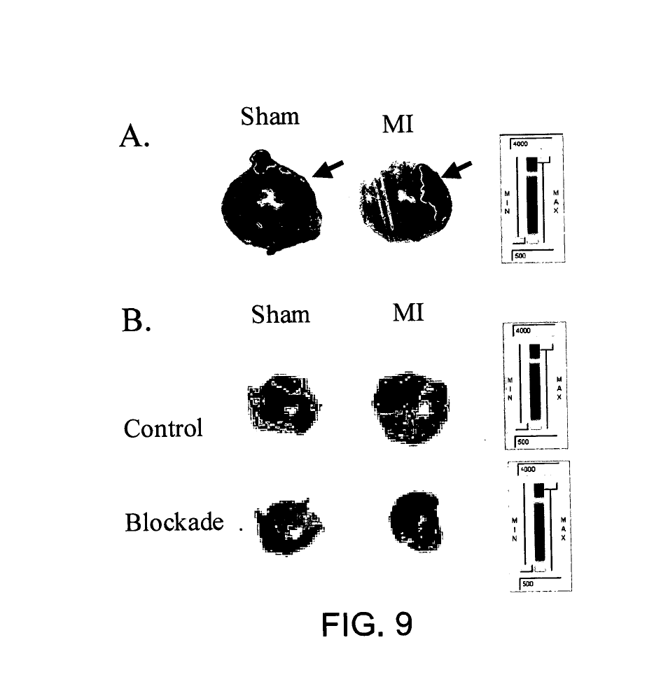

Autoradiography of the explanted hearts after ⁶⁴Cu-DOTA-VEGF₁₂₁ microPET imaging clearly shows the increase in signal observed with microPET scans, comes from the affected myocardium, as evidenced by increased activity in the anterolateral wall compared to the contralateral wall (MI/control area: 5.24±0.31, FIG. 9A, right). On the contrary, sham operated animals had no difference in activity between the two regions (MI/control area: 1.14±0.02, FIG. 9A, left).

Receptor Blocking Studies

FIG. 9B shows the results of a separate receptor autoradiography study with ¹²⁵I-VEGF₁₆₅ as radioligand. In the top panels, slides from MI hearts clearly show an increased uptake in the anterolateral wall (region of distribution of the ligated artery), which was significantly higher compared to the signal observed in sham operated animals (MI/Sham: 4.38±0.31). When samples were co-incubated with VEGF₁₂₁, in addition to ¹²⁵I-VEGF₁₆₅, (FIG. 9B, lower panels) the uptake of ¹²⁵I-VEGF₁₆₅ was significantly decreased in the MI group (MI/Sham: 0.95±0.05). These results indicate that the uptake observed in the sham and MI animals without VEGF blocking (FIG. 9B top panels) is due to the binding of ¹²⁵I-VEGF₁₆₅ to VEGF receptors (as uptake was significantly decreased when those receptors were blocked with naïve VEGF₁₂₁, and most importantly provides indirect evidence that the signal observed in the living subjects with ⁶⁴Cu-DOTA-VEGF₁₂₁ are due to an increase in VEGF receptors in MI.

VEGF Receptor Immunostaining.

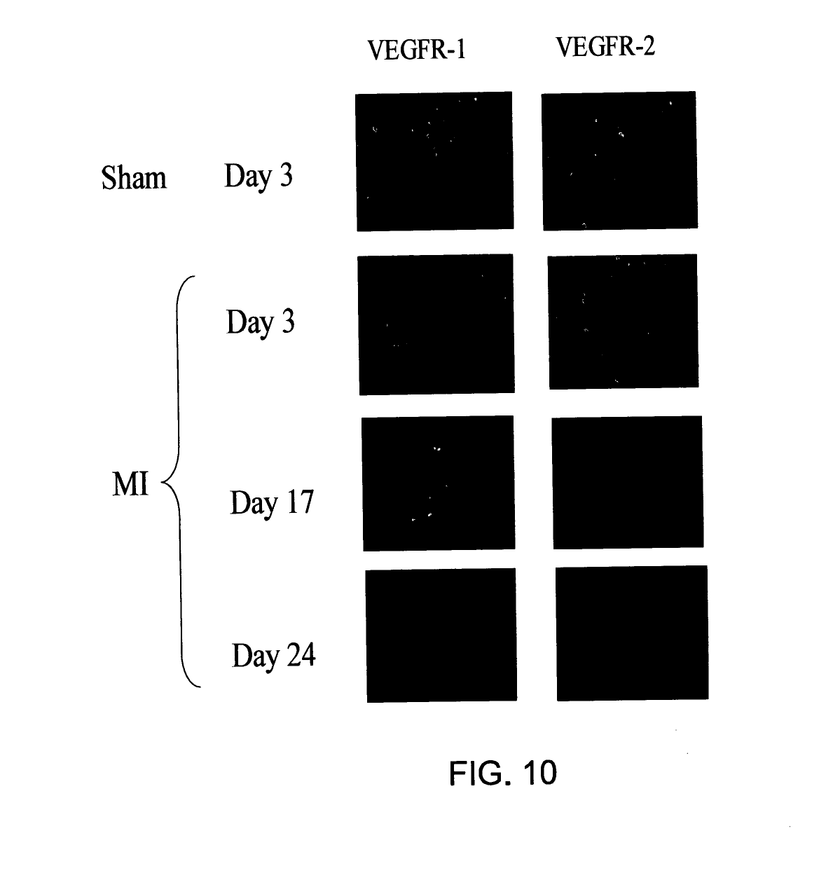

To further characterize the activation of the VEGF receptors after MI, we performed immunostaining of myocardial tissue slices for VEGF receptors-1 and -2, and observed an increase in the expression of VEGF receptors 1 and 2 after MI, compared to sham operation (FIG. 10). The expression of VEGF receptor 2 seemed to be more pronounced than VEGF receptor 1. Importantly the expression of VEGF receptors was more pronounced at day 3 and decreased over time (FIG. 10), similar to what occurred with the signal obtained with microPET.

Discussion

In the current study we have shown, for the first time, the feasibility of imaging VEGF receptors serially in the myocardium of living subjects using small animal PET. Furthermore, we imaged and described the kinetics of VEGF receptor expression after myocardial infarction. Use of molecular imaging strategies like the one presented here can provide invaluable in-vivo information regarding the pathobiology of coronary artery disease, and due to its non-invasive nature has the potential to be translated to patients.

Myocardial infarction induces changes in the microenvironment of the myocardium, and is associated with increase in the expression of inflammatory cytokines and tissue growth factors that may lead to an angiogenic response, and may play a role in the LV remodeling response observed after an MI. Over the last few years, VEGF has been observed to be one of the main growth factors activated in this state, and previous studies have shown that myocardial infarction leads to an increase in the expression of VEGF and VEGF receptors. However, in most of these studies the biological pathways were studied, either after sacrificing the animal or after obtaining a biopsy sample from a patient, and performing studies ex-vivo. In the current study we show for the first time the feasibility of serially studying biological pathways in coronary artery disease in the intact living subject. Using a non-invasive PET imaging approach, we confirmed previous observations of the activation of VEGF receptors after MI⁴, and further extended them by performing longitudinal monitoring of VEGF expression in the intact subject.

Non-invasive imaging modalities for the study of organ biology and pathobiology offer considerable advantages. Firstly, they allow us to study biology in-vivo, with minimal perturbation of the microenvironment. Secondly, it permits longitudinal monitoring of activity of the pathway of interest, which allows us to better determine the behavior of a certain pathway in the same individual under different conditions (e.g., natural progression of the disease, before and after intervention). Finally, these strategies permit translation to the clinics, permitting the study of the disease not just in an animal model but in the actual patient. Many imaging modalities have the potential for this use, including ultrasound, single photon emission computed tomography (SPECT), and PET. PET as well as SPECT have the advantage of being minimally operator dependent, resulting in higher reproducibility. In addition, due to its high energy and quantitation capabilities PET appears as the imaging modality of choice for the study of pathobiology of disease. In fact, over the years our laboratory has developed numerous novel PET probes for the study of cell biology.

⁶⁴Cu has a relatively long half life (t_{1/2}: 12.7 hours), compared to ¹⁸F (t_{1/2}:109 minutes) or ⁶⁸Ga (t_{1/2}:68 minutes), which precluded us from performing more frequent monitoring of VEGF receptor expression. In addition, use of radioactive isotopes with a relatively long half-life are less translatable to patients. Based on the results from this study (the best signal/background ratio was obtained 1 hour after probe delivery), future studies will likely benefit in using radio-isotopes with shorter half-lives. In that respect, either ⁶⁸Ga or ¹⁸F are potential alternatives as their half lives are sufficiently short to provide reasonable low radiation exposure, but long enough to allow for radio-labeling of VEGF₁₂₁.

To image VEGF receptors in this study, we used the VEGF₁₂₁ protein as the substrate for the VEGF receptor. Previous studies have shown that VEGF₁₂₁ has affinity for VEGF receptors-1 and -2. Thus, based on the in-vivo results from this study we can not conclude if there is differential activation of VEGF receptors after myocardial infarction. Furthermore, it is also possible that part of the increased uptake observed after myocardial infarction is due to an inflammatory response, which is a known response to myocardial infarction. The intact fraction of ⁶⁴Cu-DOTA-VEGF₁₂₁ is at least 75% based on analytical HPLC, with a very small fraction (3-5%) of free ⁶⁴Cu. The remaining fraction (20%) was composed of metabolites and fragments of the tracer. Although they can possibly be taken up by inflammation which may contribute to a certain fraction of the uptake in the infarct region, the majority will likely be cleared very rapidly based on our previous studies using ⁶⁴Cu-labeled small peptides. Our studies using ¹²⁵I-VEGF₁₆₅ as the detection probe, and unlabeled-VEGF₁₂₁, as the blocking agent provides indirect evidence of the specificity of this probe for the VEGF receptor. While in-vivo blockade of ⁶⁴Cu-DOTA-VEGF₁₂₁ uptake or use of a scrambled protein would have been a more physiologic measure of the specificity of this probe for the VEGF receptors, issues related to mass amount protein production and cost limitations made this a less viable alternative in this study. Also, we can not exclude the possibility that part of the in-vivo uptake may be related to protein leakage in a state of inflammation, but we doubt that is the only contributor to the increase in uptake observed after MI (as significant uptake was seen using iodinated-VEGF). Ex-vivo VEGF receptor detection also demonstrates that the signal detected in MI rats was not due to factors such as increased in-vivo regional perfusion, but rather to an actual increase in the expression of the VEGF receptors. Furthermore, the increased expression in VEGF receptors observed with immunostaining in the myocardial infarcted areas also provides indirect evidence of an association between the signal observed in PET with the up-regulation of VEGF receptors in myocardial infarction. In this study we observed an up-regulation mainly of VEGFR-2, while an up-regulation of VEGFR-1 was not as pronounced. Our observation corroborates the observations made by Li et. al. that described a dynamic and differential activation of VEGF receptors after myocardial infarction. It should also be mentioned that no partial volume correction was done in this proof-of-principle study thus its results should not be taken as absolute quantification, but rather as proof of the increased expression of VEGFR after MI.

In the present study we used a rat model of myocardial infarction, using permanent ligation of a coronary artery (Circulation. 2004; 110:685-91, which is include herein by reference). We chose this model of disease because it allows us to study the remodeling effect that infarction has on the myocardium, without confounding variables (e.g., such as reperfusion injury) that may be observed with the use variants of this model (transient coronary ligation followed by reperfusion). However, while the permanent ligation animal model is commonly used as a surrogate of coronary artery disease and MI seen in patients, it may not resemble the actual disease observed in humans. Thus, results from this study should not be extrapolated directly to what occurs in patients, but seen rather as a first and necessary step towards developing novel imaging modalities that will lead to a better understanding of coronary artery disease. Future studies from our laboratory will focus on the translation of this imaging approach to a larger animal model (e.g., porcine) as a preamble for its use in patients. This imaging approach will be the first one that to allow the study of the expression of growth factor receptors in large animals and then in patients in a non-invasive and longitudinal manner. The understanding of the pathobiology of coronary artery disease in patients is critical for the development of novel therapeutic strategies, and to better define the role of those that are currently available. Specifically, imaging of the VEGF receptor can be used to accurately study the timeline of growth factors activation in the intact patient during different stages of the disease, as well as providing insight on the effects of different therapeutic strategies (e.g., gene/cell therapy).

In summary we image and describe, for the first time in living subjects, the kinetics of VEGF receptor expression after myocardial infarction. Use of molecular imaging strategies to study the biology of CAD and its consequences will be invaluable in providing information of the activation of different biological pathways and due to its potential application to patients, will help us to better understand CAD.

### Example 3

As the Western world population continues to age, there is an increasing prevalence of peripheral arterial disease (PAD), which affects approximately 12% of adults. PAD, a manifestation of systemic atherosclerosis, is caused by atherosclerotic stenosis and/or occlusion of lower extremity arteries with consecutive muscle tissue ischemia at stress or rest resulting in two major clinical symptoms, intermittent claudication and critical limb ischemia. The natural response to muscle tissue ischemia includes the mobilization of circulating cellular elements and the upregulation of angiogenic growth factors with the corresponding binding ligands that together enable development of collateral vasculature, a process called therapeutic angiogenesis. Novel therapeutic strategies for patients refractory to conventional treatments of PAD (such as exercise training, drug therapy, bypass grafting or percutaneous interventions) aim to stimulate or augment these physiologic adaptive processes by application of angiogenic cytokines and/or gene and cell therapy protocols. Although such therapeutic strategies have shown encouraging results with increased vascular collateral growth and improved clinical symptoms in preclinical studies and early nonrandomized clinical trials, they have failed or provided unsatisfactory responses in controlled, placebo-controlled clinical trials. These conflicting results may be due to the absence of objective and quantitative measures of therapeutic angiogenesis to select for patients who would benefit most from a given specific therapeutic approaches.

Molecular imaging is a rapidly expanding field that attempts to non-invasively visualize, characterize and quantify biological processes at the cellular and subcellular level in living subjects. Direct targeted molecular imaging of specific molecular markers of therapeutic angiogenesis, such as vascular endothelial growth factor (VEGF) and its receptors (VEGFR), could provide an objective and quantitative measure for individualized monitoring of PAD therapy. VEGFR2 is one of the major regulators of therapeutic angiogenesis and activation of the VEGF/VEGFR2 axis triggers multiple downstream signaling networks that result in increased angiogenesis.

Positron emission tomography (PET) is a highly sensitive (˜10⁻¹¹-10⁻¹² moles/I) molecular imaging modality which allows objective quantification of minute differences in radiotracer uptake in different tissues of living subjects. PET imaging is increasingly being used for clinically relevant cardiovascular applications. With the recent availability of small animal PET scanners, findings from preclinical animal studies on investigational radiotracers are readily translatable from the preclinical to clinical setting.

In this study, we hypothesized that PET imaging allows noninvasive in vivo spatial, temporal, and quantitative monitoring of therapeutic angiogenesis with the use of a radiolabeled protein targeted at VEGFR2. To test this hypothesis, we longitudinally followed uptake of ⁶⁴Cu-labeled VEGF₁₂₁ (VEGF₁₂₁ protein (SEQ ID No: 1)) in murine ischemic hindlimbs over a 4-week period and quantified the effects of pro-angiogenic treadmill exercise training on radiotracer uptake.

Methods

In Vitro Assays and Cell Culture Experiments

Synthesis of VEGF₁₂₁ and VEGF_{NLS}

The gene for VEGF₁₂₁ (SEQ ID No: 1) was amplified from human umbilical vascular endothelial cells (HUVECs) via PCR and inserted into a pRSF-Duet1 prokaryotic expression vector (Novagen, San Diego, Calif.). A VEGF mutant (D63A/E64A/E67A/R82N/I83L/K84S) (SEQ ID No: 8), with decreased affinity for VEGFR2 and, to a lesser extent, also for VEGFR1 (henceforth referred to as VEGF_{NLS}) was obtained by two rounds of PCR using two pairs of overlapping primers and two flanking primers and was cloned into a pRSF-Duet1 vector. For protein expression and purification, the plasmids were transformed into BL21 (DE3) competent cells. The single clone was selected and inoculated in 50 mL of LB medium containing 50 μg/mL of kanamycin. The culture was grown at 37° C. with shaking overnight at 250 rounds per minute (rpm). Thereafter, a 10 mL culture was inoculated into 1 L of LB medium containing 50 μg/mL of kanamycin and grown at 37° C. with vigorous shaking, until the optical density of liquid medium at 600 nm (OD₆₀₀) reached 0.6-0.8. Then, Isopropyl-β-D-thiogalactopyranoside (IPTG) was added to a final concentration of 1 mM and the culture was grown for another 4 h. The cells were then harvested by centrifugation at 4000 rpm for 10 min and resuspended in cell lysis buffer (50 mM NaH₂PO₄, 300 mM NaCl, 10 mM imidazole, 20% glycerol). After sonication, the lysate was clarified by centrifugation at 12,000 rpm for 20 min, the supernatant was collected, and the protein was purified by immobilized metal affinity chromatography. The resin was washed with 50 mM NaH₂PO₄, 300 mM NaCl, 20 mM imidazole buffer and recombinant protein was eluted with buffer containing 250 mM imidazole. The eluted proteins were dialyzed against phosphate-buffered saline (PBS).

Radiolabeling of VEGF₁₂₁ and VEGF_{NLS}

⁶⁴Copper (⁶⁴Cu; t_{1/2}=12.7 h) was obtained from the University of Wisconsin, Madison and DOTA (1,4,7,10-tetraazadodecane-N,N′,N″,N′″-tetraacetic acid) was purchased from Macrocyclics, Inc. (Dallas, Tex.). The procedure for DOTA conjugation and radiolabeling VEGF (VEGF₁₂₁ and VEGF_{NLS}) has been reported elsewhere (J Nucl Med. 2006; 47:2048-2056). In brief, DOTA-VEGF was purified using a PD-10 column (GE Healthcare; Piscataway, N.J.) and concentrated by Centricon filter (Ultracel YM-10; Millipore, Bedford, Mass.). The average number of DOTA chelators per VEGF was determined using the procedure reported by Meares et al. with some modifications. Briefly, 6 μg of DOTA-VEGF in 100 μL of 0.1 M sodium acetate buffer (pH=6.5; T=40° C.) were added to a defined amount of carrier-added ⁶⁴CuCl₂ solution and the number of DOTA molecules per VEGF was calculated using the following equation: Number of DOTA molecules per VEGF=moles of (Cu²⁺)×yield/moles of (DOTA-VEGF). For ⁶⁴Cu-DOTA-VEGF₁₂₁, the radiolabeling yield was 69.7±6.3%, the number of DOTA molecules was 2.2±0.1, and the specific activity was 116.2±10.5 mCi/mg with a radiochemical purity of larger than 98% in our study. For ⁶⁴Cu-DOTA-VEGF_{NLS}, the radiolabeling yield was 53.9±9.1%, the number of DOTA molecules was 0.9±0.2, and the specific activity was 89.8±15.2 mCi/mg with radiochemical purity of larger than 98%.

Binding Assay of VEGF₁₂, and VEGF_{NLS}

Porcine aortic endothelial cells (PAE-KDR cells) stably transfected to express human VEGFR2 (KDR) and porcine aortic endothelial cells (PAE cells) not expressing VEGFR1 and VEGFR2 were cultured in Ham's F-12 medium containing 10% fetal bovine serum (Sigma-Aldrich, Saint Louis, Mo.). Using ¹²⁵I-VEGF₁₆₅ as the radioligand, receptor-binding affinity of VEGF₁₂₁ and VEGF_{NLS} for VEGFR2 was performed as described elsewhere for both PAE-KDR and PAE cells (J Med. Chem. 2005; 48:1098-1106, which is include herein by reference). To analyze the binding affinity of VEGF₁₂₁ and VEGF_{NLS}to VEGFR1, soluble VEGFR1_{D1-D6} (sVEGFR1) (Research Diagnostics, Inc., Concord, Mass.) was diluted using coating buffer (15 mM NaCO₃, 35 mM NaHCO₃, pH 9.6) and coated onto 96-well plates (NUNC, Rochester, N.Y.) overnight at 4° C. Serially diluted VEGF₁₂₁ or VEGF_{NLS} were then added to compete with the radioligand ¹²⁵I-VEGF₁₆₅. After 2 h incubation at room temperature, the plates were washed and bound ¹²⁵I-VEGF₁₆₅/sVEGFR1 complex was dissociated by acidified 0.1% SDS solution, the eluent was collected and the radioactivity was measured by a gamma counter (Cobra II Auto-Gamma; Perkin-Elmer, Wellesley, Mass.). The best-fit 50% inhibitory concentration (IC₅₀) values were calculated by fitting the data by nonlinear regression using GraphPad Prism 4.0 (GraphPad Software, Inc., San Diego, Calif.). Experiments were performed twice with triplicate samples.

Animals and Treadmill Exercise Protocol

Mouse Model of Hindlimb Ischemia

Animal protocols were approved by the Institutional Administrative Panel on Laboratory Animal Care. Unilateral hindlimb ischemia in the left leg was introduced in 4-month-old male C57BL/6J mice (n=46, Jackson Laboratories, Bar Harbor, Me.) as described elsewhere (Circulation. 2005; 112:2501-2509, which is include herein by reference). In brief, mice were anesthetized with 2% isoflurane in oxygen at 2 L/min and placed on a warming pad to maintain the body temperature at 37° C. Under sterile conditions, the femoral artery was ligated proximal and distal to the caudal femoral artery using 7-0 silk sutures (Ethicon, Somerville, N.J.). The arterial segment between the ligatures was excised. A sham procedure (preparation of arteries without ligations and excisions) was performed on the contralateral right leg in 10 mice. The skin was closed with 5-0 proline sutures (Ethicon, Somerville, N.J.). All animals were housed in a temperature-controlled animal facility with a 12-hour light/dark cycle and had free access to water and rodent chow.

Laser Doppler Hindlimb Tissue Perfusion Measurements

One day before surgery as well as at days 1, 8, 15, 22 and 29 after surgery, in vivo hindlimb tissue perfusion imaging of the mice was performed with a laser Doppler imaging system (Periscan PIM 3; Perimed AB, Järfälla, Sweden). To minimize variability of measurements, all perfusion measurements were performed under 2% isoflurane anesthesia in a temperature and light-controlled room. Animals were acclimatized for 60 minutes prior to induction of anesthesia. In addition, a rectal temperature probe was used to monitor the core temperature of the mice. The temperature of the mice was kept constant at 37° C.±0.5° C. by using a heating pad and a warming lamp. Three perfusion images were obtained in each mouse. Average hindlimb perfusion was expressed as the ratio of ischemic to non-ischemic hindlimb by drawing regions of interests over both hind limbs. Notably, the laser Doppler perfusion system primarily measures superficial blood flow, that is, skin circulation that does not necessarily directly reflect skeletal muscle perfusion (Contact Dermatitis. 2002; 46:129-140, which is include herein by reference). Therefore, the device was used to document the initial postoperative reduction in hindlimb perfusion and the recovery of perfusion over the following 4 weeks. It was not used to measure differences in skeletal muscle perfusion between mice with and without treadmill exercise training (see below).

In Vivo Assessment of Hindlimb Function and Ischemic Damage

One day before surgery as well as at days 1, 8, 15, 22 and 29 after surgery, hindlimb function of all mice was clinically assessed and graded by using a four-point grading scale (ambulatory impairment score) as described elsewhere: 4, dragging of foot; 3, no dragging of foot but no plantar flexion; 2, plantar flexion but no flexing of toes; 1, normal function with flexing of toes to resist gentle traction on the tail (Circulation. 2003; 108:205-210, which is include herein by reference). In addition, ischemic damage of hindlimbs was evaluated clinically and graded by using a five-point grading scale (tissue damage score): 5, any amputation; 4, tissue necrosis; 3, severe discoloration; 2, mild discoloration; 1, no difference compared to non-ischemic contralateral hindlimb.

Treadmill Exercise Training

A subgroup of 20 mice was exercised on a motorized, open rodent treadmill (Exer-3-6; Columbus Instruments, Columbus, Ohio). The treadmill exercise training began 3 days after surgery and was performed 5 times a week. Each training session started with a speed of 9 m/min for 3 minutes to allow the mice to acclimate to the treadmill. The speed was then increased by 3 m/min every 3 minutes until a maximum speed of 18 m/min. The training was performed until the mice were unable to keep pace (mean training time per day, 35 minutes; range, 30-40 minutes) and was executed after 7 p.m. to ensure that the exercise took place during the daily active cycle of the mice. Five mice were trained for 1 week, 5 mice for 2 weeks, 5 mice for 3 weeks, and 5 mice for 4 weeks.

Small Animal Imaging Experiments

PET Imaging

In all animals (exercised and non-exercised mice), imaging was performed on a Concorde R4 microPET system (Siemens AG, Malvern, Pa.) with the mice in the supine position. Animals were maintained under 2% isoflurane anesthesia and the hindlimbs of the animals were centered in the detector ring during data acquisition. One, 4, and 20 hours after intravenous injection of ⁶⁴Cu-VEGF₁₂₁ (mean, 238 μCi; range, 217-277 μCi) via the tail vein, a 5-minute static scan with an approximate resolution of 2 mm in each axial direction was obtained in all animals. Images were reconstructed using the ordered-subsets expectation maximization (OSEM) algorithm.

To further test the specificity of the signal coming from ⁶⁴Cu-VEGF₁₂₁ binding to mouse VEGFR2, ⁶⁴Cu-VEGF_{NLS} was administered in additional 3 non-exercised mice (day 8 after surgery) and PET scanning was performed as described above. In addition, in vivo-blocking studies was performed in additional 3 non-exercised mice (day 8 after surgery). First, 250 μg of rat anti-mouse VEGFR2 monoclonal antibody (Avas 12a1; eBioscience, San Diego, Calif.) were injected via tail-vein in each of these 3 mice. Sixty minutes after injection of the monoclonal antibodies (to allow distribution of the antibodies in the tissue) ⁶⁴Cu-VEGF₁₂₁ was injected through the tail vein and PET imaging was performed as described above in each of these 3 mice.

Image Analysis of PET images

PET images were analyzed offline by using nonproprietary PET analysis software (AMIDE version 0.8.2; http://amide.sourceforge.net). Image analysis was performed in random order by one reader who was blinded to all information about the mice (number of days after femoral artery ligation and days of treadmill exercise training). Regions of interest (ROI) encompassing both the ischemic and non-ischemic muscles of both hindlimbs were drawn. Percentage injected dose per gram (% ID/g) was calculated by means of a calibration constant obtained from scanning a cylindrical phantom in the small-animal PET scanner, assuming a tissue density of 1 g/mL, and dividing by the injected dose that was decay-corrected to the time of scanning. No corrections for partial volume or attenuation were performed.

Postmortem Analysis

Gamma Well Counting

In 3 mice, the skeletal muscles of the ischemic and non-ischemic hindlimbs were excised and weighed. The tissue radioactivity was measured with a gamma counter (Cobra II Auto-Gamma; Perkin Elmer, Wellesley, Mass.) and was corrected for background, decay time, and tissue weight.

Immunohistochemistry

After completion of PET imaging, mice were euthanized, and the hindlimb muscle tissue from both sides were transferred into embedding medium (O.C.T.; Sakura, Torrance, Calif.) and immediately frozen on dry ice. Frozen tissue slices (5-μm thickness) of the muscle tissue were fixed with cold acetone for 10 min and dried in air for 30 min. The slices were rinsed with PBS for 2 min and blocked with 10% donkey serum for 30 min at room temperature. The slices were then incubated with rat anti-mouse VEGFR2 antibody (DC101; ImClone Systems, Inc., New York, N.Y.) overnight at 4° C. and visualized using FITC-conjugated donkey anti-rat secondary antibody (1:200; Jackson ImmunoResearch Laboratories, Inc., West Grove, Pa.). For VEGFR1 staining, the slices were incubated with rabbit anti-mouse VEGFR1 antibody (1:50, Lab Vision, Fremont, Calif.) for 1 h at room temperature and visualized using Cy3-conjugated donkey anti-rabbit secondary antibody (1:200, Jackson ImmunoResearch Laboratories, Inc, West Grove, Pa.). Slices were also stained for CD31 (a marker for endothelial cells) to localize VEGFR2 expression to muscle vessels. For this purpose, the slices were incubated with rat anti-mouse CD31 antibody (1:100; BD Biosciences, San Jose, Calif.) at room temperature for 1 h and visualized with FITC-conjugated donkey anti-rat secondary antibody (1:200; Jackson ImmunoResearch Laboratories, Inc., West Grove, Pa.).

Western Blotting

Muscle tissue protein was extracted using a tissue protein extraction buffer (T-PER; Pierce Biotechnology, Inc., Rockfod, Ill.). Forty μg of protein of each muscle sample were separated by running on a NuPage Bis-Tris Gel (Invitrogen, Carlsbad, Calif.). The protein was transferred to a Nitrocellulose Membrane (Invitrogen, Carlsbad, Calif.) and blocked with 5% nonfat milk blocking buffer for 1 h at room temperature. The membrane was then incubated with a rabbit anti-mouse VEGFR2 primary antibody (1:1000; Upstate USA Inc, Charlottesville, Va.) overnight at 4° C. The washed membrane was then incubated with a horse radish peroxidase-conjugated anti-rabbit secondary antibody (1:5000; GE Healthcare, Piscataway, N.J.) for 1 h at room temperature. The Enhanced chemiluminescence (ECL) detection kit (GE Healthcare, Piscataway, N.J.) was used for exposure to X-ray film. As an internal loading control, the same membrane was stripped and incubated with anti-α-tubulin antibody. Relative VEGFR2 expression levels were quantified on western blots using ImageJ software (version 1.32; National Institutes of Health, Bethesda, Md.) after densitometric scanning of the exposed films.

Statistical Analysis

Data are given as mean ±standard deviation. The two-tailed paired and unpaired Student t-tests were used to test differences within animals (ischemic versus non-ischemic contralateral or sham operated contralateral hindlimb of mice) and between animals (exercised versus non-exercised mice; VEGF_{NLS} versus wild type VEGF₁₂₁; in vivo blocking versus non-blocking), respectively. Correlations between PET values and both Gamma counting and western blotting results were expressed using the Pearson correlation coefficient. Differences were considered significant at a P value of less than 0.05.

Results

Binding Assays Demonstrate Specific Binding Affinity of VEGF₁₂₁ and Reduced Binding of VEGF_{NLS} to VEGFR2.

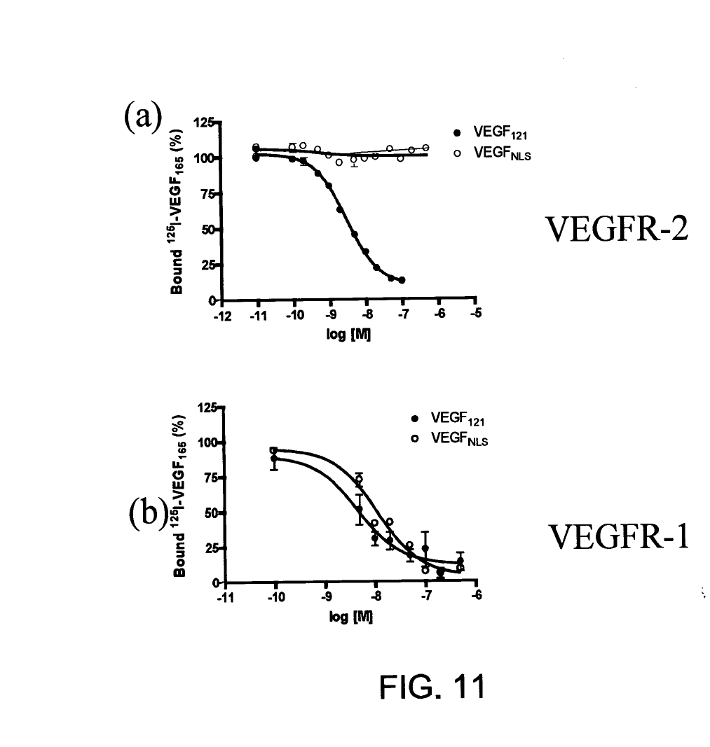

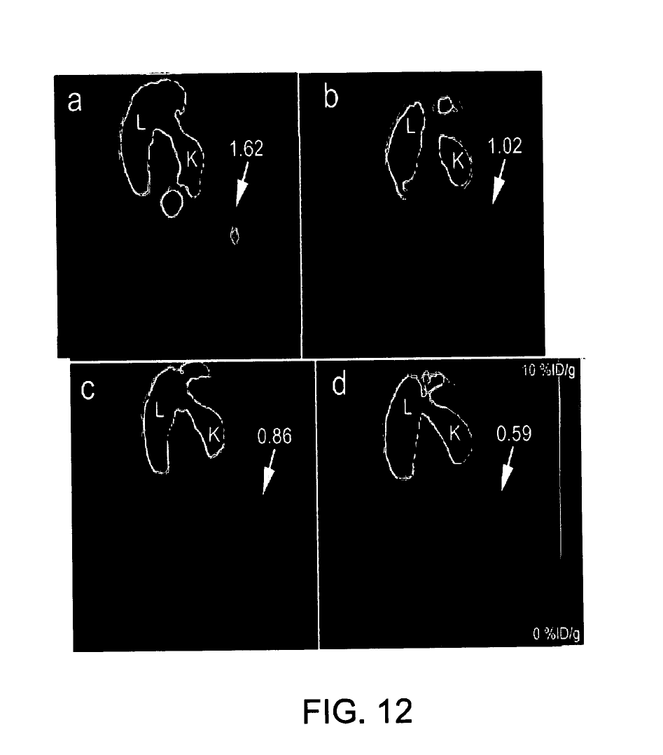

By using ¹²⁵I-VEGF₁₆₅ as a competitive radioligand, the binding affinity of wild type VEGF₁₂₁ and VEGF_{NLS} to VEGFR2 and VEGFR1 was assessed in binding experiments. Using VEGFR2-expressing cells (PAE-KDR), the IC₅₀ value for VEGF_{NLS}(10 μmol/l) was 3400 fold higher than the IC₅₀ value for VEGF₁₂₁ (2.9 nmol/l), confirming a reduced binding affinity of VEGF_{NLS} to VEGFR2 (FIG. 11a). Binding affinity of VEGF_{NLS} to VEGFR1 was reduced to a lesser extent (IC₅₀ value of 12 nmol/l for VEGF_{NLS} compared to an IC₅₀ value of 4 nmol/l for VEGF₁₂₁) (FIG. 11b). There was no significant binding of both VEGF₁₂₁ and VEGF_{NLS} to negative control cells (PAE).

Laser Doppler Imaging and Clinical Assessment Confirm Ischemia after Femoral Artery Ligation and Hindlimb Blood Flow Recovery Over Four Weeks.

To confirm successful induction of tissue ischemia after femoral artery ligation, laser Doppler imaging was performed and mice were evaluated clinically using the ambulatory impairment score and the tissue damage score. Average hindlimb tissue perfusion ratio (ischemic/non-ischemic hindlimb) decreased from 1.0±0.04 before surgery to 0.09±0.02 at day 1 after surgery, gradually recovered over the following 4 weeks, and eventually reached 0.82±0.13 at day 29 after surgery. On serial clinical assessment, a reduced functional use of the ischemic hindlimb was noted at day 1 after surgery (mean ambulatory impairment score, 1.6±0.5). Functional use of hindlimb improved by day 8, and recovered to normal levels by day 22 and 29, respectively (Table 1, Example 3). The mean clinical tissue damage score increased from 1.0 before surgery to 2.6±0.5 at day 1, and decreased to near-normal levels at day 29 after surgery (1.2±0.4) (Table 1, Example 3). Clinical ambulatory impairment and tissue damage scores were not significantly different (P>0.35) in exercised versus non-exercised mice at any time point after surgery.

TABLE 1
Example 3. Serial clinical evaluation of ischemic hindlimbs using the ambulatory impairment score and the tissue damage score
at day 0 (1 day before surgery) and at day 1, 8, 15, 22, and 29 after femoral artery ligation (5 mice at each time point).

| | Day 0 | | Day 1 | | Day 8 | | Day 15 | | Day 22 | | Day 29 | |
|---|---|---|---|---|---|---|---|---|---|---|---|---|
| | No | | No | | No | | No | | No | | No | |
| Mouse | training | Training | training | Training | training | Training | training | Training | training | Training | training | Training |

Ambulatory Impairment Score

| | | | | | | | | | | | | |
|---|---|---|---|---|---|---|---|---|---|---|---|---|
| 1 | 1 | 1 | 2 | 2 | 2 | 2 | 2 | 1 | 1 | 1 | 1 | 1 |
| 2 | 1 | 1 | 2 | 2 | 2 | 1 | 1 | 1 | 1 | 1 | 1 | 1 |
| 3 | 1 | 1 | 2 | 2 | 2 | 1 | 1 | 1 | 1 | 1 | 1 | 1 |
| 4 | 1 | 1 | 1 | 1 | 1 | 1 | 1 | 1 | 1 | 1 | 1 | 1 |
| 5 | 1 | 1 | 1 | 1 | 1 | 1 | 1 | 1 | 1 | 1 | 1 | 1 |

Tissue Damage Score

| | | | | | | | | | | | | |
|---|---|---|---|---|---|---|---|---|---|---|---|---|
| 1 | 1 | 1 | 3 | 3 | 3 | 3 | 2 | 2 | 2 | 2 | 2 | 2 |
| 2 | 1 | 1 | 3 | 3 | 3 | 3 | 2 | 2 | 2 | 1 | 1 | 1 |
| 3 | 1 | 1 | 3 | 3 | 3 | 3 | 2 | 2 | 1 | 1 | 1 | 1 |
| 4 | 1 | 1 | 2 | 2 | 2 | 2 | 2 | 2 | 1 | 1 | 1 | 1 |
| 5 | 1 | 1 | 2 | 2 | 2 | 2 | 2 | 1 | 1 | 1 | 1 | 1 |

PET Imaging Shows Significantly Higher ⁶⁴Cu-VEGF₁₂₁ Uptake in Ischemic Hindlimbs than in Non-Ischemic Hindlimbs.

At days 8, 15, 22, and 29 after ligation of the left femoral artery, PET imaging was performed after intravenous administration of ⁶⁴Cu-VEGF₁₂₁. ⁶⁴Cu-VEGF₁₂₁ uptake in ischemic hindlimbs was significantly higher (P<0.001) than in contralateral non-ischemic hindlimbs on PET imaging at days 8, 15, and 22 after surgery (FIG. 12). In ischemic hindlimbs, VEGF₁₂₁ uptake was highest at day 8 after surgery (mean, 1.62% ID/g; range, 1.23-2.4) and decreased thereafter. Twenty-nine days after femoral artery ligation, VEGF₁₂₁ uptake in ischemic hindlimbs (mean, 0.59% ID/g; range, 0.46-0.76) was not significantly different (P=0.1) compared with radiotracer uptake in the contralateral non-ischemic hindlimbs (mean, 0.61% ID/g; range, 0.40-0.64). In vivo ROI-derived ⁶⁴Cu-VEGF₁₂₁ activity correlated well with ex vivo ⁶⁴Cu gamma well counting of the excised muscle tissues (R²=0.99; P=0.02). There was no statistically significant difference (P>0.33) in radiotracer uptake when scanning at 1 h, 4 h, and 20 h after radiotracer administration.

⁶⁴Cu-VEGF₁₂₁ Uptake in Ischemic Hindlimbs is Specific Through Binding to VEGFR2.

To test whether radiotracer uptake was due to specific VEGF₁₂₁ binding to VEGFR2 in ischemic muscle tissue or non-specific distribution of radiotracer in the tissue interstitium after surgery, sham-operated hindlimbs in 10 mice were also scanned. In sham-operated hindlimbs, radiotracer uptake (mean, 0.67% ID/g; range, 0.35-0.71) was not significantly different compared with non-ischemic hindlimbs (P=0.52). To further exclude the possibility of nonspecific retention of VEGF₁₂₁ in ischemic hindlimbs, PET scanning with ⁶⁴Cu-VEGF_{NLS} was performed. Following administration of ⁶⁴Cu-VEGF_{NLS}, the mean radiotracer uptake was significantly lower (mean, 0.88% ID/g; range, 0.75-1.07) than following administration of ⁶⁴Cu-VEGF₁₂₁ (P=0.01). Radiotracer uptake was also significantly lower (P=0.03) after pre-administration of anti-VEGF2 antibodies (mean, 0.79% ID/g; range, 0.72-0.86).

Treadmill Exercise Training Increases ⁶⁴Cu-VEGF₁₂₁ Uptake in Ischemic Hindlimbs.

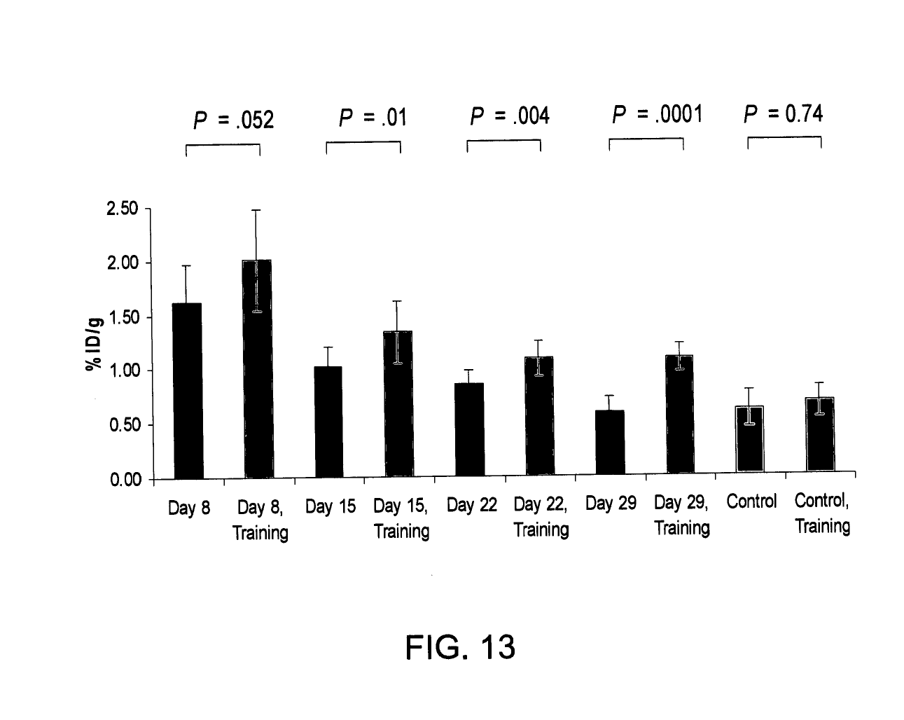

Three days after surgery, treadmill exercise training was initiated in a subgroup of 20 mice and PET imaging with ⁶⁴Cu-VEGF₁₂₁ was performed at days 8, 15, 22, and 29 after surgery. At all time points after surgery, average ⁶⁴Cu-VEGF₁₂₁ uptake in ischemic hindlimbs was higher in exercised versus non-exercised ischemic hindlimbs. ⁶⁴Cu-VEGF₁₂₁ uptake was significantly higher at days 15, 22, and 29 after surgery in exercised compared to non-exercised ischemic hindlimbs (P≦0.01) (FIG. 13).

Immunohistochemistry and Western Blotting Show Higher Amounts of VEGFR2 In Ischemic Hindlimbs than in Non-Ischemic Hindlimbs.

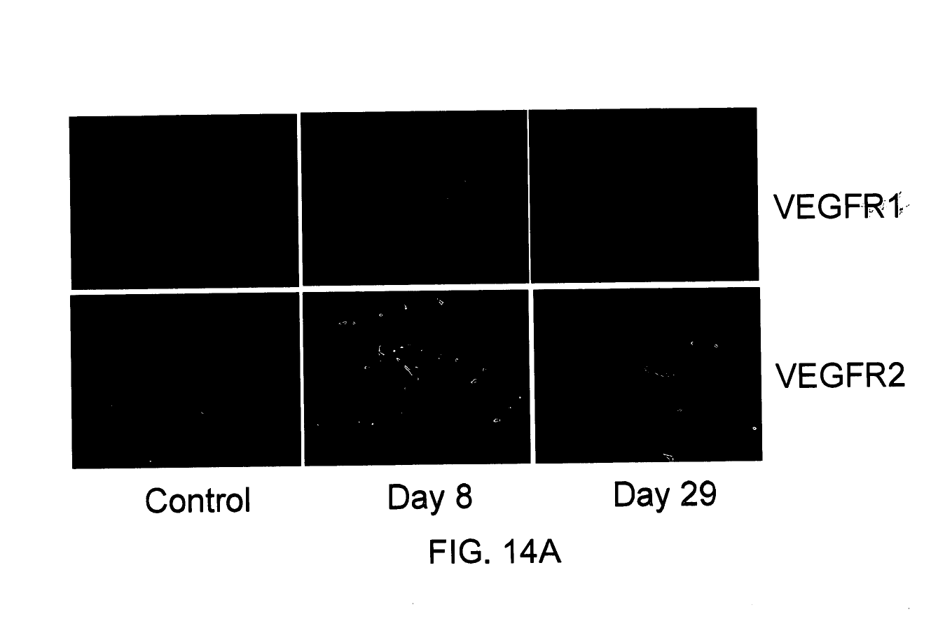

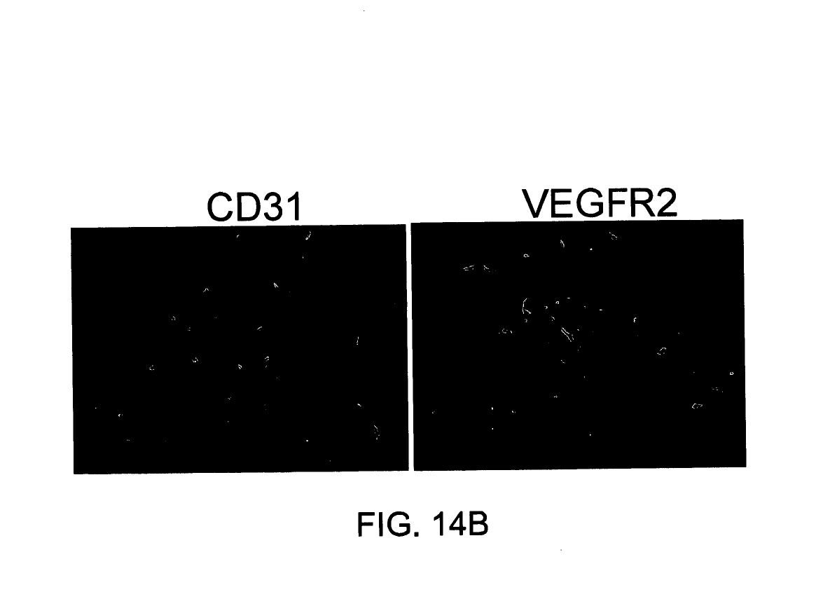

After PET imaging, mice were sacrificed and hindlimb muscle tissues were stained for VEGFR2, VEGFR1, and CD31 proteins. There was strong immunohistochemical staining for VEGFR2 in ischemic muscle tissue and almost no staining in non-ischemic control muscle tissue at day 8 after surgery (FIG. 14a). At day 29 after surgery, VEGFR2 expression levels were reduced but still higher than in control muscle tissue. Co-staining of VEGFR2 and CD31 was unsuccessful. However, visual examination of CD31 and VEGFR2 staining of slices of the same hindlimb muscle tissue suggested co-localization of VEGFR2 and CD31 on muscle vessels (FIG. 14b). There were low VEGFR1 expression levels in both ischemic and non-ischemic control muscle tissues (FIG. 14a).

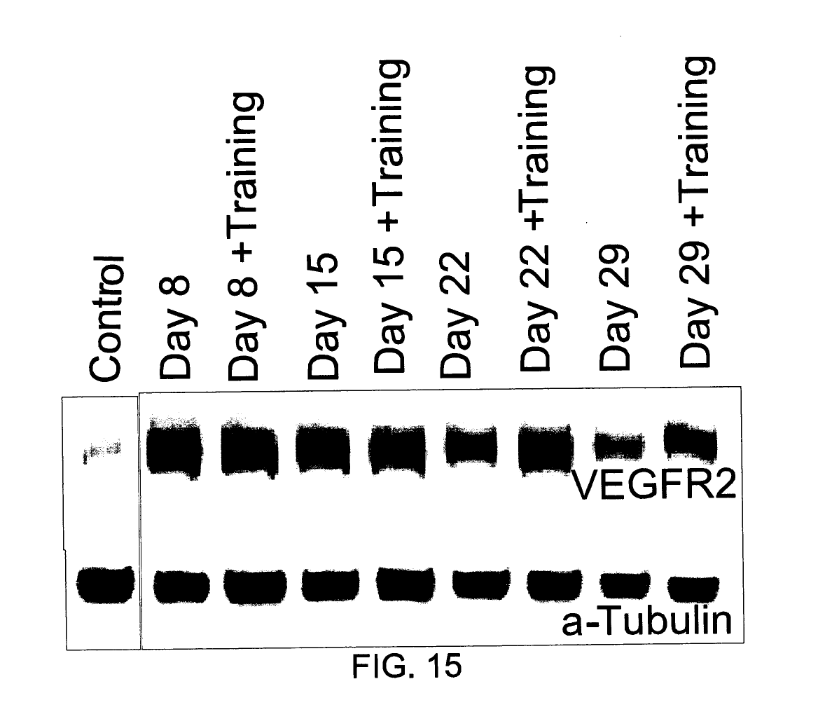

As assessed semiquantitatively by western blotting, the amount of VEGFR2 protein was higher in ischemic than in non-ischemic muscle tissue and was increased after treadmill exercise training at all time points after surgery (FIG. 15). There was a positive correlation (R²=0.76; P<0.001) between relative VEGFR2 expression levels as measured by western blotting and ⁶⁴Cu-VEGF₁₂₁ uptake as assessed by PET imaging at different time points after surgery.

Discussion

In this study we demonstrated that therapeutic angiogenesis can be monitored in murine hindlimb ischemia by PET imaging with ⁶⁴Cu-labeled VEGF₁₂₁. In addition, we showed that the effects of treadmill exercise training on spatial and temporal VEGFR2 expression levels can be quantified longitudinally by ⁶⁴Cu-VEGF₁₂₁-PET imaging. We first created a mutant VEGF protein (VEGF_{NLS}) and showed in cell culture that the mean affinity of VEGF_{NLS} to cells expressing VEGFR2 was 3400 fold lower compared with wild type VEGF₁₂₁. Cells that lack expression of VEGFR2 served as control for cell culture experiments and showed no affinity to both VEGF₁₂₁ and VEGF_{NLS}. These in vitro results suggest binding specificity between recombinant VEGF₁₂₁ and its ligand VEGFR2.

We further tested the utility of ⁶⁴Cu-VEGF₁₂₁-PET imaging in vivo to noninvasively and quantitatively image therapeutic angiogenesis in a murine hindlimb model of ischemia. Laser Doppler imaging showed a successful decrease in perfusion after ligation of the femoral artery in our mouse model with a substantial reduction of the average hindlimb tissue perfusion ratio (ischemic/non-ischemic hindlimb) after surgery. In addition, two clinical ischemia assessment scores were used to further confirm initial presence of muscle tissue ischemia after surgery in our ischemia model (Circulation. 2003; 108:205-210, which is include herein by reference). One week after induction of hindlimb ischemia, ⁶⁴Cu-VEGF₁₂₁ uptake increased substantially by a factor of 2.6 in ischemic hindlimb tissue compared with contralateral control hindlimb tissue, which highly correlated with ex vivo gamma-well counting. To further validate specific binding of ⁶⁴Cu-VEGF₁₂₁ to VEGFR2, we assessed uptake of ⁶⁴Cu-labeled VEGF_{NLS} and performed in vivo blocking studies in a subgroup of ischemic animals. In both sets of experiments, radiotracer uptake was substantially decreased as compared to wild type VEGF₁₂, and PET studies without blocking. These findings suggest specific in vivo binding of ⁶⁴Cu-VEGF₁₂₁ to VEGFR2 rather than non-specific distribution of radiotracer in the tissue interstitium after surgery. This is also consistent with our observation that radiotracer uptake was not substantially increased in sham operated hindlimbs compared with non-operated control hindlimbs. Furthermore, the lack of significant differences in ⁶⁴Cu-VEGF₁₂₁ uptake in ischemic tissue at three different time points (1 h, 4 h, and 20 h) after intravenous radiotracer administration suggests a low influence of blood perfusion differences in ischemic versus non-ischemic muscle tissue and indicates long lasting radiotracer binding to its ligand VEGFR2. Immunohistochemistry findings confirmed upregulation of VEGFR2 expression on endothelial cells of hindlimb vessels in ischemic compared with non-ischemic hindlimb muscle tissue. In addition, lack of VEGFR1 upregulation in ischemic versus non-ischemic hindlimb muscle tissue as assessed by immunohistochemical staining suggests that increased ⁶⁴Cu-VEGF₁₂₁ uptake was primarily caused by binding of VEGF₁₂₁ to VEGFR2 rather than to VEGFR1. This is also corroborated by the observation that VEGF_{NLS}, which had substantial binding affinity to VEGFR1 in vitro, did not substantially accumulate in ischemic hindlimbs in vivo.

Several studies using hindlimb ischemia models have addressed the potential of radionuclide imaging approaches for visualization and quantification of molecular markers of therapeutic angiogenesis. Up-regulation of αᵥβ₃ integrin 3 days after creation of hindlimb ischemia in mice has been shown by scintigraphic imaging of radioiodine-labeled RGD [¹²³I-c(RGD(I)yV)]. In another study, expression levels of αᵥβ₃ integrin were obtained over a 2-week period following induction of hindlimb ischemia in mice by using a gamma-camera and a ^{99m}Tc-labeled chelate-peptide conjugate containing an RGD motif. Experience with radionuclide imaging of VEGFR2 expression, however, remains very limited. Lu et al. used ¹¹¹In-labeled recombinant human VEGF₁₂₁ and a gamma-camera for visualization of VEGFR2 expression over a 2-day period in a rabbit model of hindlimb ischemia. A subtle increase in scintigraphic image counts in the ischemic hindlimb (mean, 370 cpm) could be detected as compared to non-ischemic control (mean, 280 cpm) and sham-operated (mean, 310 cpm) hindlimbs in that study. We significantly add to that study, first, by tracking expression levels of VEGFR2 over a 4-week period rather than a 2-day period only, and second, by investigating therapeutic angiogenesis by PET rather than gamma-camera imaging. PET is increasingly being used for cancer and cardiovascular imaging clinically and holds significant advantages over gamma-camera imaging in that the sensitivity for detecting molecular probes is up to two orders of magnitude higher than that for gamma-camera imaging. This may explain the much higher relative radiotracer uptake values in ischemic versus non-ischemic hindlimbs in our study as compared with the values obtained with gamma-camera imaging in the study by Lu et al.(Circulation. 2003; 108:97-103, which is include herein by reference). Our study is unique in that we addressed for the first time effects of stimulated therapeutic angiogenesis on PET tracer uptake in a murine hindlimb ischemia model using a clinically relevant and widely spread therapeutic approach of treadmill exercise training. In exercised mice, ⁶⁴Cu-VEGF₁₂₁ uptake was substantially increased as compared with non-exercised mice and reached statistically significant differences two to four weeks after initiation of exercise training. This increase in radiotracer uptake on PET imaging correlated well with VEGFR2 expression levels in mice after treadmill exercise training as measured semi-quantitatively by western blotting. In contrast, we did not observe a significant difference between exercised and non-exercised mice by the two clinical assessment scores used in our study. These findings suggest that ⁶⁴Cu-VEGF₁₂₁-PET imaging may provide a more sensitive means to objectively quantify the kinetics of VEGFR2 expression in therapeutic angiogenesis than is possible with clinical evaluation scores. Therefore, this novel approach may be used as an imaging surrogate endpoint for studying various therapeutic approaches for PAD in preclinical and ultimately maybe also in clinical studies. However, this hypothesis needs to be tested in further studies both in large animals and humans. Further studies are also warranted to address whether the use of αᵥβ₃ integrin, VEGFR2, or the combination of those two or other angiogenesis molecular markers may be the most optimal imaging targets for following therapeutic angiogenesis, both in animals and humans.

The following limitations of the study need to be addressed. Hindlimb ischemia was created in healthy mice in our study. Collateral vessel creation and thus expression of molecular markers of therapeutic angiogenesis depend on the mouse strain and may vary in older, atherosclerotic, and hyper-cholesterolemic mice. Therefore, future studies are warranted to test the utility of ⁶⁴Cu-VEGF₁₂₁-PET imaging of VEGFR2 expression in animal models, which better reflect the vascular status of older, atherosclerotic, and probably hyper-cholesterolemic patients with PAD. Furthermore, it has been shown that hindlimb perfusion in C57BL/6J mice which were used in our study recovers relatively quickly, within 4 weeks, after femoral artery ligation compared to other mouse strains. Therefore, our mouse model may reflect chronic PAD only during a limited time interval after surgical creation of ischemia. Thus, the kinetics of VEGFR2 expression levels as determined in our study cannot directly be extrapolated to other animal models of hindlimb ischemia or to patients with PAD. Finally, although a recent study has shown only small estimated radiation-absorbed doses of ⁶⁴Cu-VEGF₁₂₁ in both rats and humans, a radiotracer with a shorter half life than ⁶⁴Cu for labeling VEGF₁₂₁ would be desirable in future translatable studies in humans. This is further supported by our finding that radiotracer uptake in ischemic hindlimbs was not significantly different at 1 h, 4 h, and 20 h after intravenous administration, corroborating the use of a radiotracer with a short half life such as ¹⁸F (t_{1/2}=109.7 min) or ⁶⁸Ga (t_{1/2}=68 min).

In conclusion, the results of our study suggest that PET imaging with ⁶⁴Cu-labeled VEGF₁₂, allows noninvasive spatial visualization and quantification of VEGFR2 expression in therapeutic angiogenesis in a murine model of hindlimb ischemia. Modulation of VEGFR2 expression levels by treatment with treadmill exercise training in mice can be measured by ⁶⁴Cu-VEGF₁₂₁-PET imaging.

### Example 4

Vascular endothelial growth factor (VEGF) and VEGF receptors (VEGFRs) play important roles during neurovascular repair after stroke. In this study, we evaluated the kinetics of VEGFR expression during post-stroke angiogenesis in a rat model.

In Sprague-Dawley rats, stroke was created by permanent occlusion of the distal middle cerebral artery (dMCAO). The rats were subjected to MRI and PET scans before surgery and at 2, 9, 16, 23 days after surgery. Several tracers were used for PET, namely ¹⁸F-FDG (to measure the metabolic activity of the brain), ⁶⁴Cu-DOTA-VEGF₁₂₁ (previously validated for imaging VEGFR expression), and ⁶⁴Cu-DOTA-VEGF_{NLS} (with much lower VEGFR binding affinity than VEGF₁₂₁) (VEGF_{NLS} protein (SEQ ID No: 9)). Ex vivo immunofluorescence staining was carried out on frozen brain sections to assess VEGFR expression and vessel density (CD31) at different days after stroke. Autoradiography using ¹²⁵I-VEGF₁₆₅ (VEGF₁₆₅ protein (SEQ ID No: 4)) on frozen stroke brain tissue was also carried out to validate the in vivo imaging results.

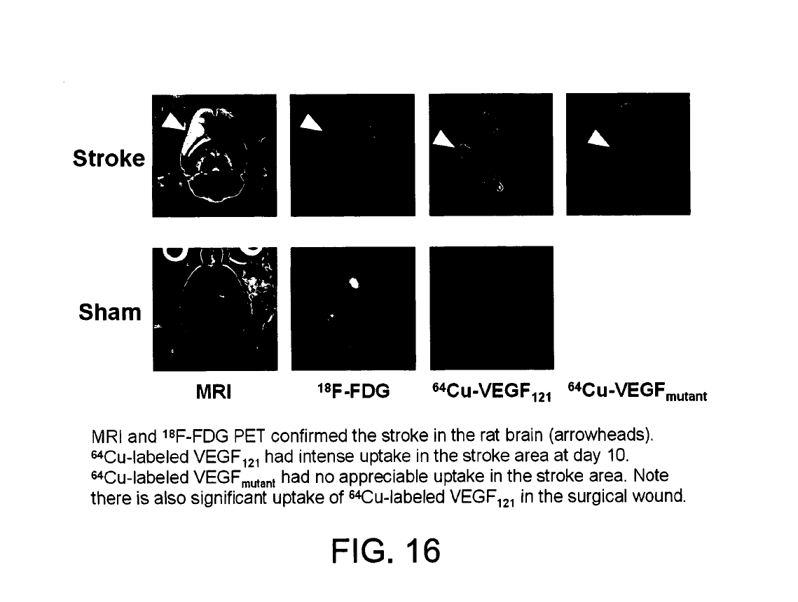

The stroke in the rat brain was confirmed by T2-weighed MRI and ¹⁸F-FDG PET (FIG. 16). The stroke size based on MRI was relatively stable over time while the size of “cold spot” in ¹⁸F-FDG PET varied to a certain extent, likely due to inflammation. ⁶⁴Cu-DOTA-VEGF₁₂₁ uptake in the stroke area peaked at about 10 days after surgery, indicating neovascularization as confirmed by ex vivo histology. VEGFR specificity of ⁶⁴Cu-DOTA-VEGF₁₂₁ uptake was confirmed by significantly lower uptake of ⁶⁴Cu-DOTA-VEGF_{NLS} in vivo and intense ¹²⁵I-VEGF₁₆₅ uptake ex vivo in the stroke area. No appreciable uptake of ⁶⁴Cu-DOTA-VEGF₁₂₁ was observed in the brain of sham operated rats, although the uptake in the surgical wound was obvious.

For the first time, we successfully evaluated the VEGFR expression kinetics noninvasively in a stroke rat model. Monitoring VEGFR expression in vivo after stroke is clinically relevant and may be translated into the clinic to determine the right timing for stroke therapy and to monitor the therapeutic efficacy by imaging post-stroke angiogenesis.

### Example 5

Tumor growth depends on angiogenesis, the formation of new blood vessels. One of the most extensively studied angiogenesis-related signaling pathways is the vascular endothelial growth factor (VEGF)/VEGF receptor (VEGFR) interactions. The VEGF family is composed of seven members with a common VEGF homology domain: VEGF-A, VEGF-B, VEGF-C, VEGF-D, VEGF-E, VEGF-F, and placenta growth factor (PIGF). VEGF-A is a dimeric, disulfide-bound glycoprotein existing in various homodimeric isoforms that differ not only in their molecular weight, but also in their biological properties. The angiogenic actions of VEGF are mainly mediated via two endothelium-specific receptor tyrosine kinases, Flt-1/FLT-1 (VEGFR-1) and Flk-1/KDR (VEGFR-2). It is now generally accepted that VEGFR-1 is critical for physiologic and developmental angiogenesis and its function varies with the stages of development, the states of physiologic and pathologic conditions, and the cell types in which it is expressed. VEGFR-2 is the major mediator of the mitogenic, angiogenic, and permeability-enhancing effects of VEGF. Over-expression of VEGF and/or VEGFRs have been implicated as poor prognostic markers in various clinical studies. Agents that prevent VEGF-A binding to its receptors, antibodies that directly block VEGFR-2, and small molecules that inhibit the kinase activity of VEGFR-2, thereby block growth factor signaling, have all been reported. Development of VEGFR-targeted molecular imaging probes could serve as a new paradigm for the assessment of anti-angiogenic therapeutics and for better understanding of the role and expression profile of VEGFR(s) in many angiogenesis-related diseases.

We have labeled wild-type VEGF₁₂₁ (VEGF₁₂₁ protein (SEQ ID No: 1)) with ⁶⁴CU (t_{1/2}=12.7 h) for PET imaging of tumor angiogenesis and VEGFR expression. MicroPET imaging revealed rapid, specific, and prominent uptake of ⁶⁴Cu-DOTA-VEGF₁₂₁ (˜15% ID/g) in highly vascularized small U87MG tumors with high VEGFR-2 expression, but significantly lower and sporadic uptake (˜3% ID/g) in large U87MG tumors with low VEGFR-2 expression. For this tracer, the highest uptake was in the kidneys. In a follow-up study, a VEGFR-2 specific fusion protein VEGF₁₂₁/rGel (rGel denotes plant toxin gelonin) was used to treat orthotopic glioblastoma in a mouse model whereby the therapeutic efficacy was monitored by bioluminescence imaging (BLI), magnetic resonance imaging (MRI), and PET. More recently, VEGF₁₂₁ was site-specifically labeled with ⁶⁴Cu. It was found that although PEGylation improved the pharmacokinetics, the kidney uptake was still very high. Several VEGF-based tracers have also been reported for SPECT and optical imaging of tumor angiogenesis, most of which had high kidney uptake.

In these reports, VEGFR imaging was achieved using VEGF-A based tracers. All VEGF-A isoforms bind to both VEGFR-1 and VEGFR-2. The kidney is usually a dose-limiting organ because it has high VEGFR-1 expression, which can take up VEGF-A based tracers. In our previous study, we found that the uptake of ⁶⁴Cu-DOTA-VEGF₁₂₁ in the kidneys was mainly due to VEGFR-1 binding (and some renal clearance), while the tumor uptake was mainly related to VEGFR-2 expression. The high renal uptake thus may limit its future clinical applications. Alanine-scanning mutagenesis has been used to identify the amino acids in VEGF₁₆₅ that mediates its VEGFR binding. Arg⁸², Lys⁸⁴, and His⁸⁶, located in a hairpin loop, were found to be critical for binding VEGFR-2, while some negatively charged residues, Asp⁶³, Glu⁶⁴, and Glu⁶⁷, were associated with VEGFR-1 binding. As VEGF₁₂₁ is a soluble, non-heparin-binding variant that contains the full range of biological and receptor-binding activities of the larger variants, in this study we developed a D63AE64AE67A mutant of VEGF₁₂₁ (VEGF_{DEE}) (SEQ ID No: 8), which was then conjugated with DOTA and labeled with ⁶⁴Cu for PET imaging of VEGFR-2 expression.

Material and Methods

Reagents

The polymerase chain reaction (PCR) reagents (Promega), molecular biology enzymes (New England Biolabs), bacterial strains, pRSF-Duet-1 bacterial expression plasmids (Novagen), Ni-NTA agarose (QiaGen), vector containing the human VEGFR-1 gene and neomycin resistance (GeneCopoeia Inc.), tissue culture reagents (GIBCO), Lipofectin (Invitrogen), and antibodies for immunofluorescence staining (Jackson ImmunoResearch Laboratories, Inc.) are all commercially available. All other chemicals were obtained from either Sigma or Fisher Scientific. ⁶⁴Cu was purchased from University of Wisconsin-Madison.

Cell Lines and Animal Model

The 4T1 murine breast cancer cell line was obtained from American Type Culture Collection (ATCC). Porcine aortic endothelial (PAE) cells were transfected with human VEGFR-1 (FLT-1) encoding vector using Lipofectin-mediated procedure according to the manufacturer's protocol. The clone with the highest VEGFR-1 expression level based on FACS analysis was selected for cell binding assay. PAE cells that expressed human VEGFR-2 (PAE/KDR) was a kind gift from Dr. Joseph M. Backer at SibTech, Inc. Both PAE/FLT-1 and PAE/KDR cell lines were cultured in Ham's F-12 medium containing 10% fetal calf serum. Animal experiments were carried out according to a protocol approved by Stanford University Institutional Animal Care and Use Committee (IACUC). Female BALB/c mice were obtained from Harlan (Indianapolis, Ind.). The 4T1 tumor model was generated by injection of 2×10⁶ 4T1 cells in 100 μl PBS into the front left flanks of the mice. Two weeks after inoculation, when the tumors reached the size of about 100-150 mm³, the mice were subjected to microPET imaging and biodistribution studies.

Generation of VEGF₁₂₁ and its Mutant

A pair of primers were designed and synthesized for VEGF₁₂₁: catatggcacccatggcagaaggagga (sense) (SEQ ID NO:30) and ctcgagtcagtggtgatgatggtgatgggatccccgcctcggcttgtcac

(anti-sense) (SEQ ID NO: 31). Overlap extension PCR was performed to generate its D63AE64AE67A mutant (VEGF_{DEE}) (SEQ ID No: 8). Two simple mutagenic PCRs were performed, each using one overlapping primer and one flanking primer. The primers used were: catatggcacccatggcagaaggagga

(flanking sense) (SEQ ID NO: 30), ctcgagtcagtggtgatgatggtgatgggatccccgcctcggcttgtcac

(flanking anti-sense) (SEQ ID NO: 31), catatggcacccatggcagaaggagga

(overlapping primer 1) (SEQ ID NO: 32), and gtgcaatgcagcaggcctggcatgtgtgcc (overlapping primer 2) (SEQ ID NO: 33). An NdeI and XhoI restriction site was incorporated into each respective flanking primer. The two PCR products were then annealed and extended.

The resulting full-length DNA was identified by DNA sequencing. The clones with desired mutations were amplified, and the genes encoding VEGF₁₂, and VEGF_{DEE} were released by NdeI and XhoI double digestion and ligated into pRSF-Duet1 expression vector (pretreated with the same restriction endonuclease enzymes). The plasmids containing the genes of interest were transfected into E. coli BL21 (DE3). The single clone was cultured in 5 ml LB medium containing 30 μg/ml kanamycin at 37° C. with constant shaking, and the overnight cultures were inoculated into 2 I LB medium and cultured until the OD₆₀₀ nm was between 0.6 and 1.0. Protein expression was then induced by adding isopropyl-β-D-thiogalactopyranoside (IPTG) to a final concentration of 0.5 mM and cultured for 4 h. The cells were harvested and the cell pellet was resuspended in lysis buffer (20 mM NaH₂PO₄, 10 mM Tris.HCl, 10 mM imidazole, 300 mM NaCl, pH 8.0) for sonication. The lysate was then centrifuged at 12,000 rpm for 20 minutes. The supernatant was collected and incubated with Ni-NTA agarose at 4° C. for 1 hr. The His-tagged recombinant protein was eluted from the agarose by elution buffer (20 mM NaH₂PO₄, 10 mM Tris.HCl, 300 mM imidazole, 300 mM NaCl, pH 8.0), dialysed against PBS, and stored at −80° C. for later use.

DOTA Conjugation and ⁶⁴Cu-Labeling

Detailed procedures for DOTA (DOTA denotes 1,4,7,10-tetra-azacylododecane N,N′,N″,N′″-tetraacetic acid) conjugation and ⁶⁴Cu-labeling were described herein (J Nucl Med 2006; 47:2048-2056, which is include herein by reference). The final concentrations of DOTA-VEGF₁₂, and DOTA-VEGF_{DEE} were determined based on UV absorbance at 280 nm using unconjugated VEGF₁₂₁ of known concentrations as the standards. ⁶⁴CuCl₂ (37 MBq) was diluted in 300 μl of 0.1 M sodium acetate buffer (NaOAc, pH=6.5), and added to 10 μg of DOTA-VEGF₁₂₁ or DOTA-VEGF_{DEE}. The reaction mixture was incubated for 1 h at 40° C. with constant shaking. ⁶⁴Cu-DOTA-VEGF₁₂₁ and ⁶⁴Cu-DOTA-VEGF_{DEE} were purified by size exclusion chromatography using PBS as the mobile phase.

Receptor Binding Assay and Functional Analysis

The detailed procedure of competitive cell binding assay has been described herein. VEGFR-1 and VEGFR-2 binding affinity of VEGF₁₂₁, VEGF_{DEE}, and the DOTA conjugates was evaluated by PAE/FLT-1 and PAE/KDR cell binding assays using ¹²⁵I-VEGF₁₆₅ (VEGF₁₆₅ protein (SEQ ID No: 4)) as the radioligand. The best-fit IC₅₀ values were calculated by fitting the data by nonlinear regression using GraphPad Prism (GraphPad Software, Inc.). Experiments were carried out with triplicate samples.

To determine the functional activity of VEGF₁₂₁, VEGF_{DEE}, and the DOTA conjugates, PAE/KDR cells were stimulated by serial concentrations of each protein for 5 minutes and the cell lysate was then immuno-blotted by anti-phosphorylated VEGFR-2 antibody.

MicroPET Studies

PET imaging of 4T1 tumor-bearing mice was performed on a microPET R4 rodent model scanner (Concorde Microsystems, Knoxville, Tenn.) as described earlier (Neoplasia 2005; 7:271-279; J Nucl Med 2007; 48:304-310, which is include herein by reference). Each mouse was injected with about 5-8 MBq of the tracer (−2-3 μg of protein) via tail vein and scanned at 1, 4, and 20 h post-injection (p.i.). For each microPET scan, three-dimensional regions-of-interest (ROIs) were drawn over the tumor, liver, kidneys, and muscle on decay-corrected whole-body coronal images. The average radioactivity concentration within a tumor or an organ was obtained from mean pixel values within the ROI volume, which were converted to counts per milliliter per minute by using a pre-determined conversion factor. Assuming a tissue density of 1 g/ml, the counts per milliliter per minute were converted to counts per gram per minute, and then divided by the injected dose to obtain an image ROI-derived percentage injected dose per gram of tissue (% ID/g).

Biodistribution Studies

Tumor-bearing mice injected with ⁶⁴Cu-DOTA-VEGF₁₂₁ or ⁶⁴Cu-DOTA-VEGF_{DEE} were euthanized and blood, tumor, major organs and tissues were collected and wet weighed. The radioactivity in the tissue was measured using a y-counter (Packard). For each mouse, the radioactivity of the tissue samples was calibrated against a known aliquot of the injectate and normalized to a body weight of 20 g [Eur J Nucl Med Mol Imaging 2007; Nat Nanotechnol 2007; 2:47-52]. Values are presented as mean ±SD for a group of three animals.

Immunofluorescence Staining

Frozen tissue slices (5 μm thick) were fixed with cold acetone for 10 minutes and dried in the air for 30 minutes. After rinsing with PBS and blocked with 10% donkey serum for 30 minutes at room temperature, the slices were incubated with rat anti-mouse VEGFR-2 antibody overnight at 4° C. and visualized using Cy3-conjugated donkey anti-rat secondary antibody. For VEGFR-1 staining, the tissue slices were incubated with rabbit anti-mouse VEGFR-1 antibody at room temperature for 1 hour and visualized with Cy3-conjugated donkey anti-rabbit secondary antibody [J Nucl Med 2006; 47:2048-2056].

Statistical Analysis

Quantitative data were expressed as mean ±SD. Means were compared using one-way analysis of variance (ANOVA) and student's t-test. P values less than 0.05 were considered statistically significant.

Results

Receptor Binding Affinity and Functional Activity of VEGF₁₂₁ and VEGF_{DEE}

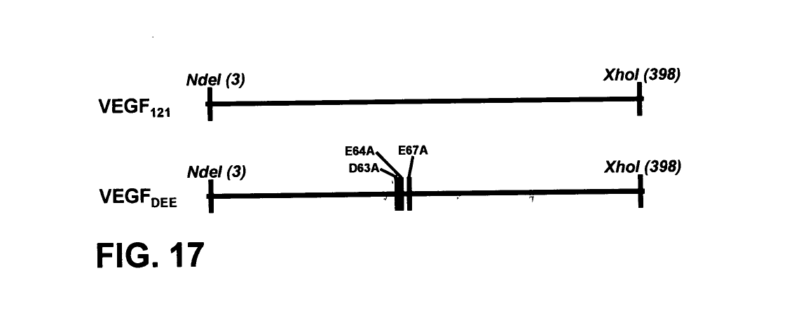

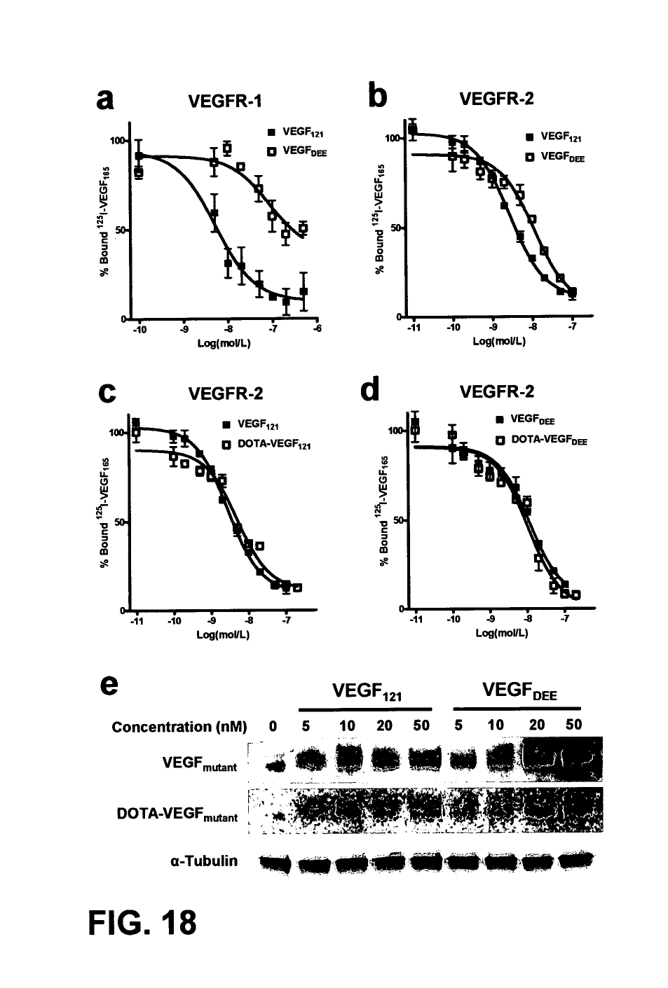

Two proteins were prepared for this study: VEGF₁₂₁ and the triple mutant at the 63, 64, and 67 positions (VEGF_{DEE}) (FIG. 17). The His-tag containing proteins were purified to homogeneity. Cell binding assay was then carried out to determine the VEGFR-1 and VEGFR-2 binding affinity (FIGS. 18a and 18b). For VEGFR-1, the IC₅₀ values of VEGF₁₂₁ and VEGF_{DEE} were 4.2 nM and 78.1 nM, respectively. For VEGFR-2, the IC₅₀ values of VEGF₁₂₁ and VEGF_{DEE} were 2.9 nM and 11.7 nM, respectively. Taken together, VEGF_{DEE} has about 20 fold lower VEGFR-1 binding and only 4-fold lower VEGFR-2 binding affinity when compared to VEGF₁₂₁. The binding affinity of VEGF_{DEE} to VEGFR-2 is about 7 fold higher than VEGFR-1.

The number of DOTA molecules per monomeric VEGF₁₂₁ and VEGF_{DEE} was measured to be 2.1±0.1 and 2.2±0.1, respectively (n=3) [J Nucl Med 2006; 47:2048-2056]. DOTA-VEGF₁₂₁ and DOTA-VEGF_{DEE} have similar VEGFR-2 binding affinity (IC₅₀ values are 5.0 and 10.3 nM, respectively) as the non-DOTA-conjugated proteins (FIGS. 18c and 18d). Functional assay based on Western blot analysis of VEGF₁₂₁, VEGF_{DEE}, and their DOTA-conjugates using PAE/KDR cells showed similar phosphorylated VEGFR-2 expression (FIG. 18e), indicating that all four proteins are functionally active at concentrations greater than 10 nM with a consistent single protein band at 200 kDa.

⁶⁴Cu-Labeling

⁶⁴Cu-labeling, including the final purification, took 80±10 min (n=5). The radio-labeling yield was similar for both DOTA-VEGF₁₂₁ and DOTA-VEGF_{DEE} (>85% based on 10 μg of protein per 37 MBq of ⁶⁴Cu). The specific activity of ⁶⁴Cu-DOTA-VEGF₁₂₁ and ⁶⁴Cu-DOTA-VEGF_{DEE} was also similar at about 3.0 GBq/mg protein.

MicroPET Studies

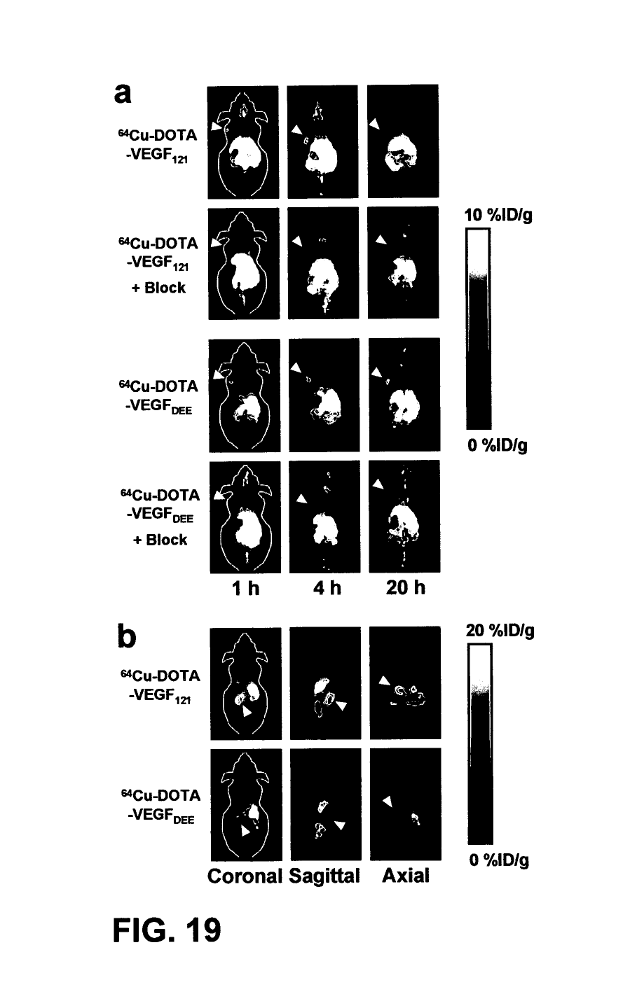

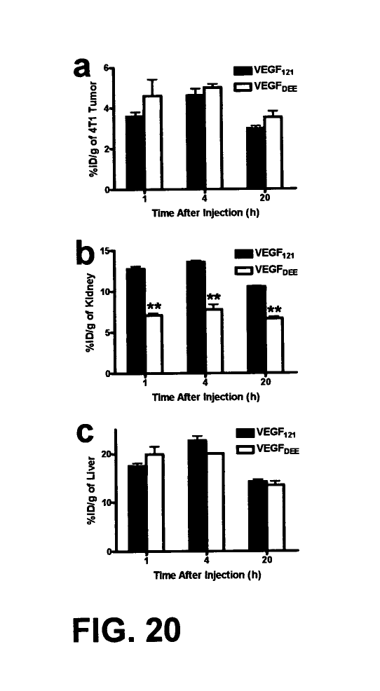

⁶⁴Cu-DOTA-VEGF₁₂₁ and ⁶⁴Cu-DOTA-VEGF_{DEE} were injected intravenously into 4T1 tumor-bearing mice and microPET scans were carried out at various time points p.i. The coronal slices that contained the tumor are shown in FIG. 19a. The tumor uptake of ⁶⁴Cu-DOTA-VEGF₁₂₁ was 3.6±0.4, 4.6±0.5, and 3.0±0.2% ID/g at 1, 4, and 20 h p.i., respectively (FIG. 20a). The tumor uptake of ⁶⁴Cu-DOTA-VEGF_{DEE} was slightly higher at 4.6±1.4, 5.0±0.3, and 3.5±0.5% ID/g at 1, 4, and 20 h p.i., respectively. The uptake levels of ⁶⁴Cu-DOTA-VEGF₁₂₁ and ⁶⁴Cu-DOTA-VEGF_{DEE} in the major organs were similar except for the kidneys.

The kidney uptake between the two tracers differed significantly at all the time points examined (12.8±0.5, 13.5±0.3, and 10.5±0.2% ID/g for ⁶⁴Cu-DOTA-VEGF₁₂₁ and 7.1±0.5, 7.8±1.1, and 6.7±0.4% ID/g for ⁶⁴Cu-DOTA-VEGF_{DEE} at 1, 4, and 20 h p.i., respectively; FIG. 20b; P<0.01 in all cases). The coronal, sagittal and axial slices containing the kidney of mice injected with ⁶⁴Cu-DOTA-VEGF₁₂₁ or ⁶⁴Cu-DOTA-VEGF_{DEE} are shown in FIG. 19b. The liver uptake for both tracers was prominent (>10% ID/g at all time points; FIG. 20c), likely due to the hepatic clearance of the two tracers and possible trans-chelation. This phenomenon was also observed in our previous studies (J Nucl Med 2006; 47:2048-2056, which is include herein by reference). In summary, ⁶⁴Cu-DOTA-VEGF_{DEE} had significantly lower kidney uptake while the tumor and other major organ uptake was comparable to ⁶⁴Cu-DOTA-VEGF₁₂₁.

Blocking Experiment and Biodistribution Studies

To confirm the VEGFR specificity in vivo, blocking experiments were carried out where 200 μg of VEGF₁₂₁ was co-injected with the tracers into tumor-bearing mice. The tumor uptake was significantly reduced to <2.0% ID/g, confirming the VEGFR specificity of both ⁶⁴Cu-DOTA-VEGF₁₂₁ and ⁶⁴Cu-DOTA-VEGF_{DEE} (FIG. 19a).

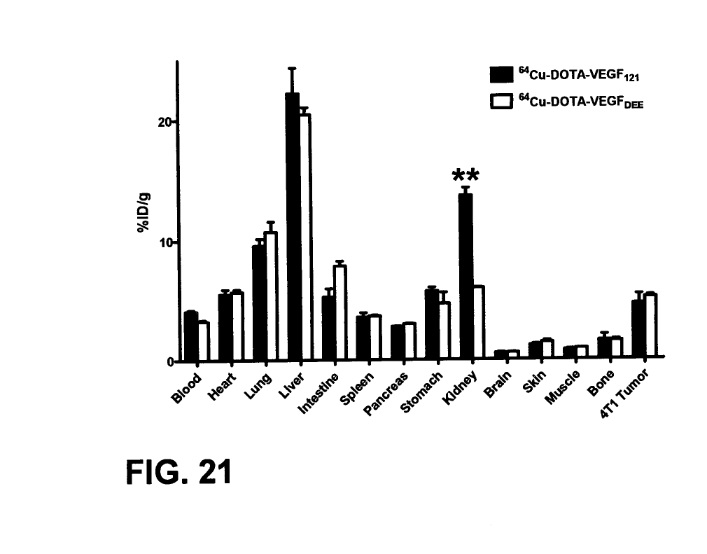

Biodistribution studies were carried out on another group of 4T1 tumor-bearing mice (FIG. 21). Besides the liver and kidneys, the lung also had significant tracer uptake. The lung has been shown to express both VEFGR-1 and VEGFR-2, and significant uptake in the lung was also observed in previous reports of VEGF-based tracers. The quantification results obtained from biodistribution studies and PET scans were similar for the 4T1 tumor and most organs, confirming that quantification of non-invasive microPET scans is a true reflection of the biodistribution of ⁶⁴Cu-DOTA-VEGF₁₂₁ and ⁶⁴Cu-DOTA-VEGF_{DEE} in vivo. Based on biodistribution results, the only organ with significantly different uptake between the two tracers was the kidney (P<0.01), consistent with the observation from non-invasive microPET scans.

Immunofluorescence Staining

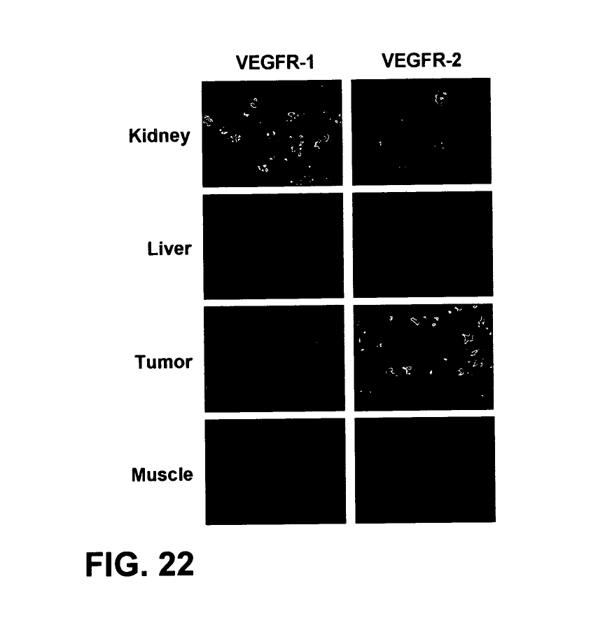

After the radioactivity in the microPET studies has decayed, the mice were sacrificed and the 4T1 tumor and major organs (liver, kidneys, and muscle) were harvested for VEGFR-1 and VEGFR-2 staining (FIG. 22). VEGFR-1 expression was low in most organs except for the kidney, which is most likely responsible for the observed high kidney uptake of ⁶⁴Cu-DOTA-VEGF₁₂₁. The 4T1 tumor had high VEGFR-2 expression (and low VEGFR-1 expression), suggesting that the tumor uptake of the two tracers was mainly due to VEGFR-2 binding. There was also measurable level of VEGFR-2 in the kidneys, mostly on the small vessels but not on the large, mature vessels. These findings demonstrate that both ⁶⁴Cu-DOTA-VEGF₁₂₁ and ⁶⁴Cu-DOTA-VEGF_{DEE} accumulated similarly in the tumor due to VEGFR-2 binding, whereas ⁶⁴Cu-DOTA-VEGF_{DEE} had significantly lower kidney uptake due to its much lower VEGFR-1 binding affinity compared to ⁶⁴Cu-DOTA-VEGF_{121.}

Discussion

We succeeded in our goal to develop a VEGFR-2 specific PET tracer in this study. In vitro, VEGF_{DEE} had only slightly lower VEGFR-2 binding affinity but much lower VEGFR-1 binding affinity when compared with VEGF₁₂₁. In vivo, ⁶⁴Cu-DOTA-VEGF_{DEE} had significantly lower kidney uptake while the tumor uptake was only slightly higher than ⁶⁴Cu-DOTA-VEGF₁₂₁. Blocking studies and immunofluorescence staining also confirmed the VEGFR-2 specificity of ⁶⁴Cu-DOTA-VEGF_{DEE.}

VEGFR-2 is the target of many anti-angiogenic therapies and, by general acceptance, it is more functionally important than VEGFR-1 in many diseases including cancer. The ability to image VEGFR-2 in vivo by PET using mutant VEGF-based tracers, as we demonstrated here, can be a valuable tool for evaluating patients with a variety of malignancies, particularly those undergoing anti-angiogenic therapies that target VEGFR-2. Two separate domains of VEGF interact with VEGFR-1 and VEGFR-2. Alanine-scanning mutagenesis has revealed that Asp⁶³, Glu⁶⁴, and Glu⁶⁷ are required for the binding of VEGF to VEGFR-1. Indeed, mutation at these three positions dramatically reduced the VEGFR-1 binding affinity and only slightly affected the VEGFR-2 binding. The VEGFR-2 specificity of ⁶⁴Cu-DOTA-VEGF_{DEE} was successfully reflected in both in vitro and in vivo experiments. To further improve the VEGFR-2 specificity and completely ablate the VEGFR-1 binding for future studies, other mutants could be generated for selection of VEGFR-2 specific mutants with desirable in vivo pharmacokinetics and tumor targeting efficacy.

Significantly lower uptake of the ⁶⁴Cu-DOTA-VEGF_{DEE} in the kidneys equates lower renal toxicity. Based on the serial microPET imaging results, the tumor uptake at 1 h p.i. was already high. Thus, shorter-lived isotopes can be tested in the future to further reduce toxicity to this radiosensitive organ. ⁶⁸Ga (t_{1/2}: 68 min) is a suitable candidate and it can be readily labeled through DOTA-chelation. To reduce the liver uptake (likely due to both hepatic clearance and possible trans-chelation), other chelators such as 1,4,8,11-tetraazacyclotetradecane-1,4,8,11-tetraacetic acid (TETA) may be tested in the future. Recently, a cross-bridge ligand (CB-TE2A) has been reported to be more stable than DOTA or TETA in vivo. However, the harsh labeling condition for CB-TE2A may not be suitable for protein-based tracers such as VEGF_{DEE}. Moreover, while CB-TE2A is primarily used for ⁶⁴Cu (or other radioisotopes of Cu)-chelation, DOTA is a universal chelator which can complex a wide variety of imaging and therapeutic radioisotopes. The same DOTA conjugate can therefore be employed for both imaging and therapeutic applications with the use of the appropriate radioisotope. In addition, peptide or small molecule-based tracers may be developed for VEGFR-2 imaging, which may give higher throughput due to much their faster clearance rates. High affinity VEGFR-2 binding peptides have been reported using phage display and may be investigated in the future.

Imaging VEGF and VEGFR expression are both important for diagnosis and the monitoring of anti-angiogenic treatment efficacy. Due to the soluble and dynamic nature of VEGF proteins, imaging VEGF expression has not been very well studied and is more challenging than imaging VEGFR expression. Almost exclusively, the strategy used for VEGF imaging uses either reporter gene approaches or anti-VEGF antibodies. It was found that antibody distribution and clearance were quite heterogeneous not only between and within patients but also between and within individual tumors, suggesting that in future clinical studies, approaches employing intra-patient dose escalation or more precisely defined patient cohorts would be more ideal. Examining tumor in the same animals or patients with both VEGF- and VEGFR-targeted tracers may give important insights about the expression kinetics of VEGF and VEGFR. Moreover, studying such combinations will shed light on the relevant disease mechanisms based on VEGF/VEGFR signaling.

### CONCLUSION

We have developed a VEGFR-2 specific PET tracer, ⁶⁴Cu-DOTA-VEGF_{DEE} (a mutant of VEGF₁₂₁), for imaging VEGFR-2 expression in vivo. It has comparable tumor targeting capability as ⁶⁴Cu-DOTA-VEGF₁₂₁ but much lower renal toxicity. This tracer may be translated clinically as a valuable tool for imaging tumor angiogenesis and monitoring anti-angiogenic treatment efficacy.

### Example 6

### Site-Specific Labeling

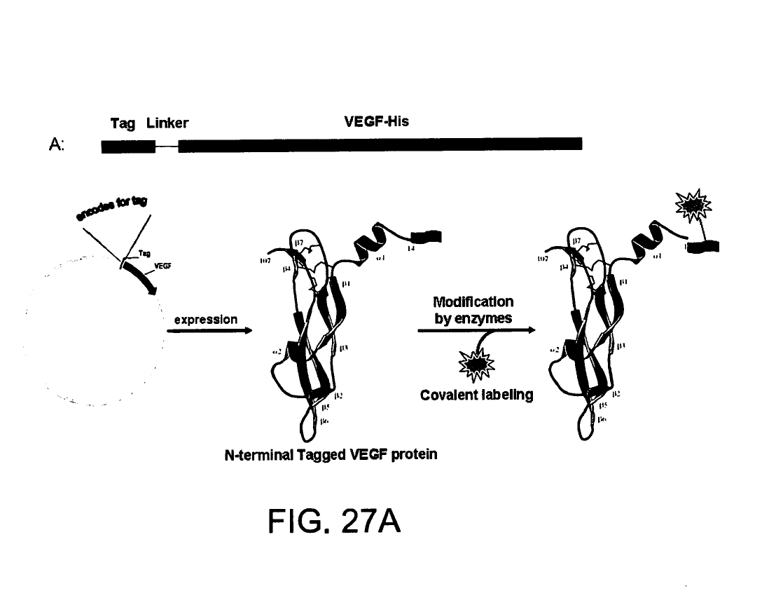

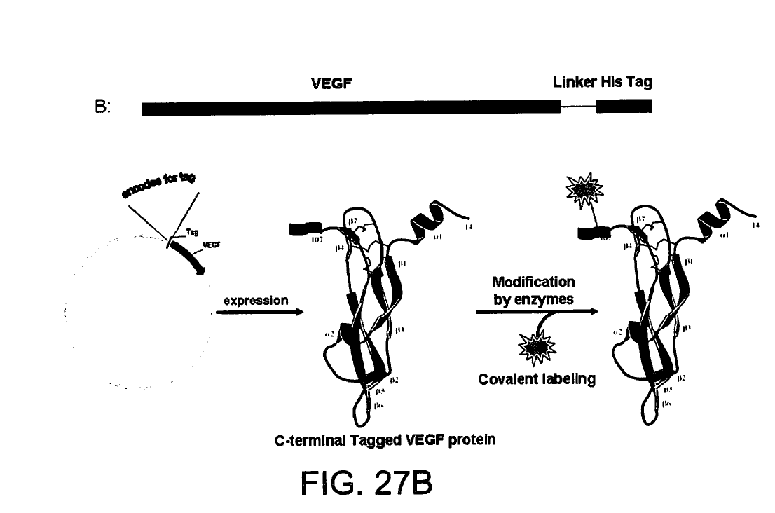

Recombinant DNA construction of protein fusions in which the protein segment has a dedicated site for labeling or could be recognized by a coenzyme ligase fused to a protein of interest, could have distinct advantages over conventional protein fusions and chemical or mutagenesis-based site-specific modification of target protein. In this example, we present examples of fusion proteins for site-specific PET labeling applications. Embodiments of possible site-specific labeling through fusion protein approach could include, but are not limited to: biotin holoenzyme synthetases (BHS) based biotinylation (Avi-tag) (SEQ ID No. 26), Sfp phosphopantethinyl transferase-based site-specific modification (ybbR-tag) (SEQ ID No. 27), formylglycine-generating enzyme-based site-specific modification (FGE-tag) (SEQ ID No. 28), and Cys-tag (SEQ ID No. 29). FIG. 27 illustrates a general scheme of site-specific labeling using embodiments of the present disclosure.

As described in more detail below, fusion proteins based on VEGF₁₂₁ (SEQ ID No: 1) and VEGF_{DEE} (SEQ ID No: 8) can include: VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25).

In addition, fusion proteins can also include: Avi-VEGF₁₄₅, VEGF₁₄₅-Avi, ybbR-VEGF₁₄₅, VEGF₁₄₅-ybbR, FGE-VEGF₁₄₅, VEGF₁₄₅-FGE, Cys-VEGF₁₄₅, VEGF₁₄₅-Cys, Avi-VEGF₁₄₈, VEGF₁₄₈-Avi, ybbR-VEGF₁₄₈, VEGF₁₄₈-ybbR, FGE-VEGF₁₄₈, VEGF₁₄₈-FGE, Cys-VEGF₁₄₈, VEGF₁₄₈-Cys, Avi-VEGF₁₆₅, VEGF₁₆₅-Avi, ybbR-VEGF₁₆₅, VEGF₁₆₅-ybbR, FGE-VEGF₁₆₅, VEGF₁₆₅-FGE, Cys-VEGF₁₆₅, VEGF₁₆₅-Cys, Avi-VEGF₁₈₃, VEGF₁₈₃-Avi, ybbR-VEGF₁₈₃, VEGF₁₈₃-ybbR, FGE-VEGF₁₈₃, VEGF₁₈₃-FGE, Cys-VEGF₁₈₃, VEGF₁₈₃-Cys, Avi-VEGF₁₈₉, VEGF₁₈₉-Avi, ybbR-VEGF₁₈₉, VEGF₁₈₉-ybbR, FGE-VEGF₁₈₉, VEGF₁₈₉-FGE, Cys-VEGF₁₈₉, VEGF₁₈₉-Cys, Avi-VEGF₂₀₆, VEGF₂₀₆-Avi, ybbR-VEGF₂₀₆, VEGF₂₀₆-ybbR, FGE-VEGF₂₀₆, VEGF₂₀₆-FGE, Cys-VEGF₂₀₆, and VEGF₂₀₆-Cys.

(A) Avi-Tag

Biotin-dependent carboxylases are a class of enzymes that undergo post-translational modification in which the biotin moiety is covalently linked to a single lysine residue via an amide bond. This two-step reaction, which is summarized in the following equations, is catalyzed by a class of enzymes termed the biotin holoenzyme synthetases (BHS)
biotin+ATP→biotinyl-5′-AMP+PPi (1)
biotinyl-5′-AMP+apocarboxylase→holocarboxylase+AMP (2)
In the first step, the enzyme catalyzes formation of an activated intermediate, biotinyl-5′-adenylate (bio-5′-AMP) from biotin and ATP. Subsequent nucleophilic attack at the activated carboxylate of biotin results in formation of the amide between the biotin moiety and the target lysine residue. The biotinylation reaction is highly specific, with only the biotin-dependent carboxylases serving as substrates in vivo. BirA is the Escherichia coli enzyme biotin ligase and can recognize linear sequence for site-specific biotinylation. The size of these tags range from 123-amino acid (PSTCD) to 15-amino acid peptide called the acceptor peptide (AP). These BirA substrate peptides have been used by a number of investigators to produce biotinylated fusion proteins for a variety of purposes. A truncation series of chemically synthesized peptides allowed identification of a 14 residues peptide as the minimal substrate for BirA. The amino acid sequence of this tag is: GLNDIFEAQKIEWH. Although this 14-mer works quite well, we select a slightly extended 15-mer, termed the Avi-tag, (GLNDIFEAQKIEWHE) (SEQ ID No: 26) that is consistently biotinylated at a rate slightly better than that of the natural substrate, for constructing the Avi-tagged VEGF fusion proteins for PET imaging purpose.
Primer Design

A fusion gene encoding VEGF₃₋₁₂₁-Avi fusion protein (SEQ ID No: 9) for site-specific biotinylation of VEGF₃₋₁₂₁ has been constructed. First, we designed and synthesized the following primers to append Avi-tag to the C-terminus of VEGF₁₂₁ for site-specific biotinylation:

Sense Primer

(SEQ ID No: 34)

ccgagttaatgatgatgatgatgatgggatccttcgtgccattcgatttt
ctgagcctcgaagatgtcgttcagaccggatccccgcctcggcttg

Antisense Primer

(SEQ ID No: 35)

ccgagttaatgatgatgatgatgatgggatccttcgtgccattcgataga
ctgagcctcgaagatgtcgttcagaccggatccccgcctcggcttg

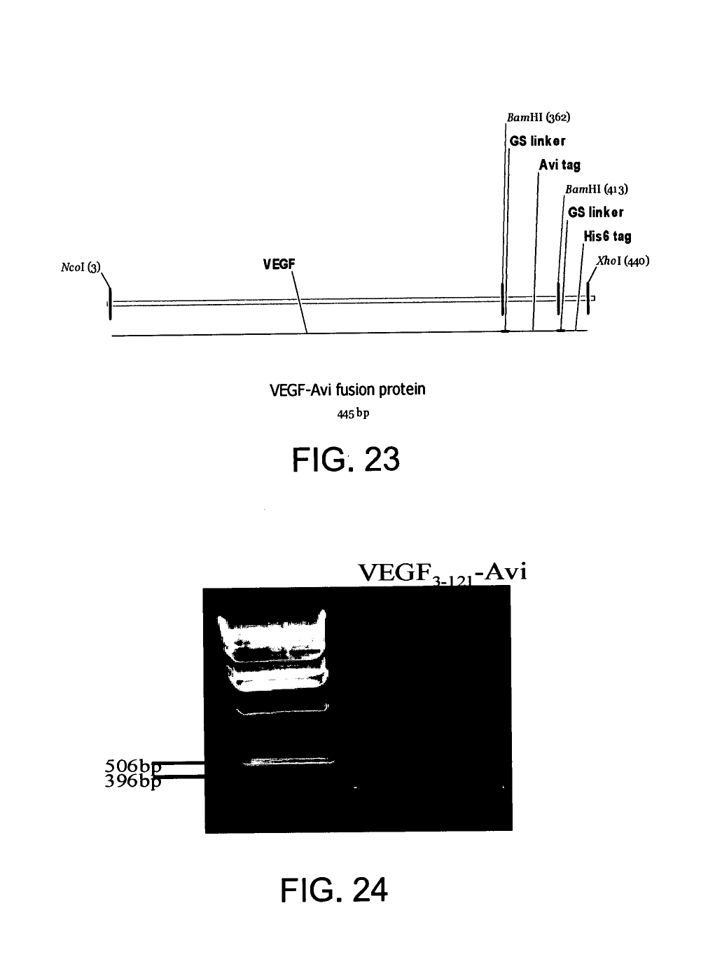

In the sense primer, the intrinsic NcoI recognition site (underlined) was used for subcloning; in the antisense primer, a GS linker was inserted between the VEGF₁₂₁ and Avi tag, XhoI recognition site (underlined) was incorporated. The VEGF₃₋₁₂₁-Avi fusion gene structure is illustrated in FIG. 23.

PCR Reaction

The gene encoding VEGF₃₋₁₂₁-Avi was generated by PCR using pRSF-VEGF as template.

Reaction mixture set up:

Gently vortex and briefly centrifuge all solutions after thawing, then add into a thin-walled PCR tube, on ice:

| Reagent | VEGF₃₋₁₂₁-Avi | |
|---|---|---|
| Sterile deionized water | 10 | μL |
| 2× GreenMaster Mix | 25 | μL |
| Sense primer (1 μM) | 5 | μL |
| Antisense primer (1), 1 μM | 5 | μL |
| Antisense primer (2), 1 μM | — | |
| Template (5 μg/ml) | 5 | μL |

Cycling Conditions:

First denaturation for 2 min at 95° C., then 30 cycles: denatured at 95° C. for 30 sec, annealing at 55° C. for 30 sec and extending at 72° C. for 30 sec. After the last cycle, the samples were incubated at 72° C. for 10 min to fill-in the protruding ends of newly synthesized PCR products.

PCR Results:

FIG. 24 illustrates the argrose electrophoresis of PCR products. The PCR product s were analyzed using 1% argrose gel, two bands corresponding to VEGF₃₋₁₂₁-Avi (SEQ ID No: 9) with the expected size (445 bp) were clearly visualized. Then the PCR products were cloned into pTOPO-TA vector (Invitrogen) for DNA sequencing.

Subclone to pET15b Expression Vector

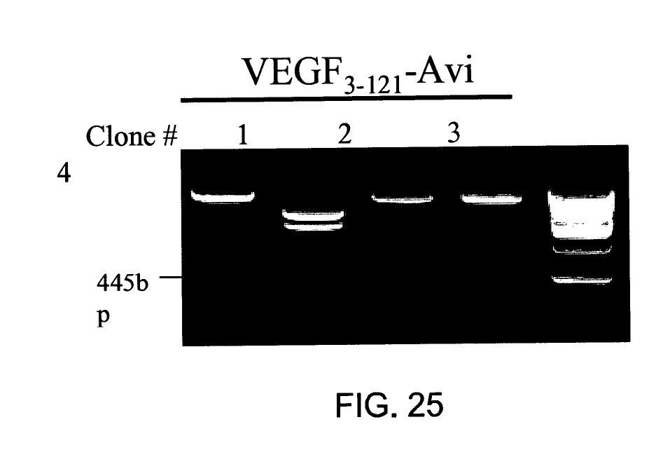

The clone with correct sequence encoding VEGF₃₋₁₂₁-Avi was subsequently cloned into prokaryotic expression vector pET15b. The constructs were identified by endonuclease restriction enzyme digestion. The results are shown in FIG. 25. In particular, FIG. 25 illustrates the identification of pET-VEGF₃₋₁₂₁-Avi (SEQ ID No: 9) using NcoI and XhoI digestion. The clone#2 of pET-VEGF₃₋₁₂₁-Avi carried the fusion gene encoding VEGF₃₋₁₂₁-Avi protein.

VEGF₃₋₁₂₁-Avi protein has been expressed and purified and site-specifically biotinylated with BirA at the C-terminus of the VEGF protein, which can be labeled with PET isotopes via suitably radiolabeled avidin or streptavidin.

(B) ybbR-Tag

The Sfp phosphopantetheinyl transferase covalently transfers 4′-phosphopantetheinyl (Ppant) groups from CoA to conserved serine residues on peptide carrier protein (PCP) and acyl carrier protein (ACP) domains in nonribosomal peptide synthetases (NRPSs) and polyketide synthases (PKSs) in Bacillus subtilis. Although the substrate for Sfp must contain CoA, the enzyme is otherwise quite tolerant of a range of small molecules attached to CoA. In fact, Sfp transfers small molecules of diverse structures from CoA to the conserved serine on the PCP or ACP domain.

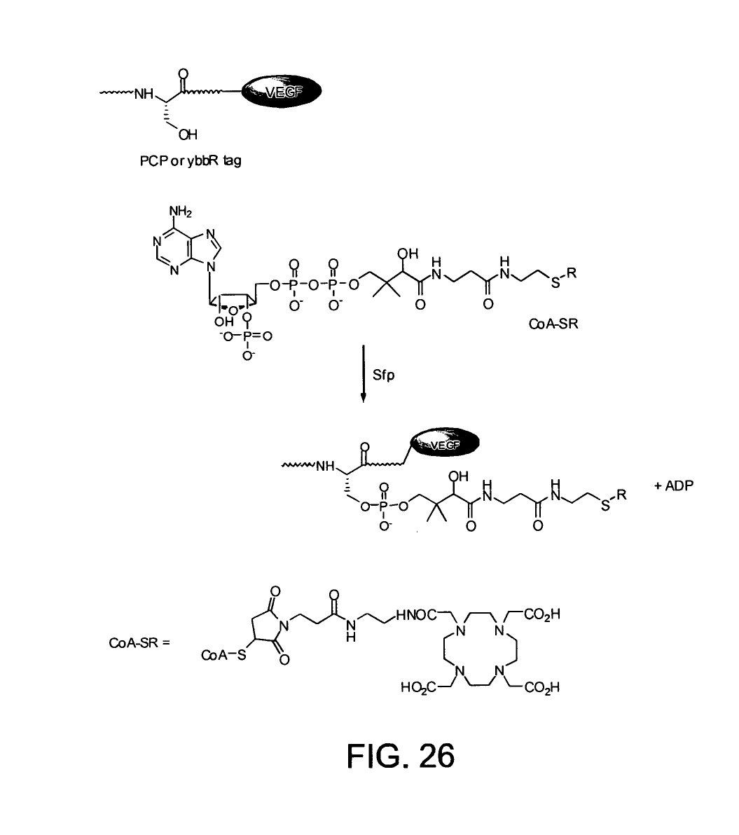

For example and shown in FIG. 26, the ybbR tag, a short (11-residue) peptide (DSLEFIASKLA, SEQ ID NO: 27), was found to be an efficient substrate for Sfp-catalyzed VEGF protein labeling, thereby replacing the full-length PCP or ACP domain for the construction of smaller fusions of the target protein. It was so named because part of its sequence was derived from the ybbR ORF in the B. subtilis genome. The site of Sfp-catalyzed ybbR tag labeling was mapped to the underlined Ser residue, and the ybbR tag was found to have a strong tendency for adopting an α-helical conformation in solution. ybbR tag can be fused to the N or C terminus of VEGF protein for site-specific protein labeling by Sfp. The short size of the ybbR tag and its compatibility with various target proteins, the broad substrate specificity of Sfp for labeling the ybbR tag with small-molecule probes of diverse structures, and the high specificity and efficiency of the labeling reaction make Sfp-catalyzed ybbR tag labeling an attractive tool for expanding protein structural and functional diversities by posttranslational modification.

(C) FGE-Tag

Most sulfatases contain an aldehyde-bearing formylglycine residue within their active site that is required for catalytic activity. The formylglycine residue is produced by co- or post-translational modification of a conserved cysteine residue found within the “sulfatase motif”. This motif contains a highly conserved CXPXR submotif, wherein X is usually serine, threonine, alanine or glycine. Formylglycine-generating enzyme (FGE) oxidizes cysteine to formylglycine and has recently been identified in eukaryotes and prokaryotes. Interestingly, FGE recognizes its substrate mainly by its primary sequence. This means that a small peptide tag based on the sulfatase motif, called the “aldehyde tag”, might direct formation of formylglycine independent of its context. Furthermore, many organisms have endogenous FGE activities, suggesting that this post-translational modification system could serve as a general means for engineering proteins for site-specific labeling. In this example, a 6-amino-acid tag (LCTPSR) (SEQ ID No: 28) containing only the core conservative residues was incorporated onto VEGF protein. Sulfatase oxidizes cysteine within the FGE-tag into formylglycine, which can then be site-specific labeled with aminooxy- or hydrazide-functionalized PET isotopes.

(D) Cys-Tag

Introducing N-terminus or C-terminus cysteine residue (SEQ ID No: 29) allows site-specific labeling of VEGF protein with thioreactive PET labels, such as the maleimide-based reagents and the haloacetic-based reagents.

Sequence Listings

One or more, may be from UniProtKB/Swiss-Prot entry protein database (http://ca.expasy.org/uniprot/P15692)

VEGF₁₂₁, SEQ ID No: 1 (P15692-7)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKCDK
PRR
VEGF₁₄₅, SEQ ID No: 2 (P15692-6)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKKSV
RGKGKGQKRKRKKSRYKSWSVCDKPRR
VEGF₁₄₈, SEQ ID No: 3 (P15692-5)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQENPCG
PCSERRKHLFVQDPQTCKCSCKNTDSRCKM
VEGF₁₆₅, SEQ ID No: 4 (P15692-4)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQENPCG
PCSERRKHLFVQDPQTCKCSCKNTDSRCKARQLELNERT
CRCDKPRR
VEGF₁₈₃, SEQ ID No: 5 (P15692-3)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKKSV
RGKGKGQKRKRKKSRPCGPCSERRKHLFVQDPQTCKCS
CKNTDSRCKARQLELNERTCRCDKPRR
VEGF₁₈₉, SEQ ID No: 6 (P15692-2)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKKSV
RGKGKGQKRKRKKSRYKSwSVPCGPCSERRKHLFVQDP
QTCKCSCKNTDSRCKARQLELNERTCRCDKPRR
VEGF₂₀₆, SEQ ID No: 7 (P15692)
APMAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKKSV
RGKGKGQKRKRKKSRYKSwSVYVGARCCLMPwSLPGPH
PCGPCSERRKHLFVQDPQTCKCSCKNTDSRCKARQLELN
ERTCRCDKPRR
VEGFR-2 specific mutant VEGF_{DEE},
SEQ ID No: 8
MAPMAEGGGQNHHEVVKFMDVYQRSYCHPIETLVDIFQE
YPDEIEYIFKPSCVPLMRCGGCCNAAGLACVPTEESNITM
QIMRIKPHQGQHIGEMSFLQHNKCECRPKKDRARQEKCD
KPRRGS
VEGFR-1 specific mutant VEGF_{NLS},
SEQ ID No: 9
MAPMAEGGGQNHHEVVKFMDVYQRSYCHPIETLVDIFQE
YPDEIEYIFKPSCVPLMRCGGCCNDEGLECVPTEESNITM
QIMNLSPHQGQHIGEMSFLQHNKCECRPKKDRARQEKCD
KPRR
VEGF₃₋₁₂₁-Avi, SEQ ID No: 10
MAEGGGQNHHEVVKFMDVYQRSYCHPIETLVDIFQEYPD
EIEYIFKPSCVPLMRCGGCCNDEGLECVPTEESNITMQIM
RIKPHQGQHIGEMSFLQHNKCECRPKKDRARQEKCDKPR
RGSGLNDIFEAQKIEWHE
Avi-VEGF₃₋₁₂₁, SEQ ID No: 11
MGLNDIFEAQKIEWHEGSAEGGGQNHHEVVKFMDVYQR
SYCHPIETLVDIFQEYPDEIEYIFKPSCVPLMRCGGCCND
EGLECVPTEESNITMQIMRIKPHQGQHIGEMSFLQHNKCE
CRPKKDRARQEKCDKPRR
VEGF_{DEE}-Avi, SEQ ID No: 12
MAEGGGQNHHEVVKFMDVYQRSYCHPIETLVDIFQEYPD
EIEYIFKPSCVPLMRCGGCCNAAGLACVPTEESNITMQIM
RIKPHQGQHIGEMSFLQHNKCECRPKKDRARQEKCDKPR
RGSGLNDIFEAQKIEWHE
Avi-VEGF_{DEE}, SEQ ID No: 13
MGLNDIFEAQKIEWHEGSAEGGGQNHHEVVKFMDVYQR
SYCHPIETLVDIFQEYPDEIEYIFKPSCVPLMRCGGCCNA
AGLACVPTEESNITMQIMRIKPHQGQHIGEMSFLQHNKCE
CRPKKDRARQEKCDKPRR
VEGF₃₋₁₂₁-ybbR, SEQ ID No: 14
MAEGGGQNHHEVVKFMDVYQRSYCHPIETLVDIFQEYPD
EIEYIFKPSCVPLMRCGGCCNDEGLECVPTEESNITMQIM
RIKPHQGQHIGEMSFLQHNKCECRPKKDRARQEKCDKPR
RGSDSLEFIASKLA
ybbR-VEGF₃₋₁₂₁, SEQ ID No: 15
MDSLEFIASKLAGSAEGGGQNHHEVVKFMDVYQRSYCHP
IETLVDIFQEYPDEIEYIFKPSCVPLMRCGGCCNDEGLEC
VPTEESNITMQIMRIKPHQGQHIGEMSFLQHNKCECRPKK
DRARQEKCDKPRR
VEGF_{DEE}-YbbR, SEQ ID No: 16
MAEGGGQNHHEVVKFMDVYQRSYCHPIETLVDIFQEYPD
EIEYIFKPSCVPLMRCGGCCNAAGLACVPTEESNITMQIM
RIKPHQGQHIGEMSFLQHNKCECRPKKDRARQEKCDKPR
RGSDSLEFIASKLA
YbbR-VEGF_{DEE}, SEQ ID No: 17
MDSLEFIASKLAGSAEGGGQNHHEVVKFMDVYQRSYCHP
IETLVDIFQEYPDEIEYIFKPSCVPLMRCGGCCNAAGLAC
VPTEESNITMQIMRIKPHQGQHIGEMSFLQHNKCECRPKK
DRARQEKCDKPRR
VEGF₃₋₁₂₁-FGE, SEQ ID No: 18
MAEGGGQNHHEVVKFMDVYQRSYCHPIETLVDIFQEYPD
EIEYIFKPSCVPLMRCGGCCNDEGLECVPTEESNITMQIM
RIKPHQGQHIGEMSFLQHNKCECRPKKDRARQEKCDKPR
RGSLCTPSR
FGE-VEGF₃₋₁₂₁, SEQ ID No: 19
MLCTPSRGSAEGGGQNHHEVVKFMDVYQRSYCHPIETLV
DIFQEYPDEIEYIFKPSCVPLMRCGGCCNDEGLECVPTEE
SNITMQIMRIKPHQGQHIGEMSFLQHNKCECRPKKDRAR
QEKCDKPRR
VEGF_{DEE}-FGE, SEQ ID No: 20
MAEGGGQNHHEVVKFMDVYQRSYCHPIETLVDIFQEYPD
EIEYIFKPSCVPLMRCGGCCNAAGLACVPTEESNITMQIM
RIKPHQGQHIGEMSFLQHNKCECRPKKDRARQEKCDKPR
RGSLCTPSR
FGE-VEGF_{DEE}, SEQ ID No: 21
MLCTPSRGSAEGGGQNHHEVVKFMDVYQRSYCHPIETLV
DIFQEYPDEIEYIFKPSCVPLMRCGGCCNAAGLACVPTEE
SNITMQIMRIKPHQGQHIGEMSFLQHNKCECRPKKDRAR
QEKCDKPRR
VEGF₃₋₁₂₁-Cys, SEQ ID No: 22
MAEGGGQNHHEVVKFMDVYQRSYCHPIETLVDIFQEYPD
EIEYIFKPSCVPLMRCGGCCNDEGLECVPTEESNITMQIM
RIKPHQGQHIGEMSFLQHNKCECRPKKDRARQEKCDKPR
RGSC
Cys-VEGF₃₋₁₂₁, SEQ ID No: 23
CGSAEGGGQNHHEVVKFMDVYQRSYCHPIETLVDIFQEY
PDEIEYIFKPSCVPLMRCGGCCNDEGLECVPTEESNITMQ
IMRIKPHQGQHIGEMSFLQHNKCECRPKKDRARQEKCDK
PRR
VEGE_{DEE}-CYS, SEQ ID No: 24
MAEGGGQNHHEVVKFMDVYQRSYCHPIETLVDIFQEYPD
EIEYIFKPSCVPLMRCGGCCNAAGLACVPTEESNITMQIM
RIKPHQGQHIGEMSFLQHNKCECRPKKDRARQEKCDKPR
RGSC
CYS-VEGF_{DEE}, SEQ ID No: 25
CGSAEGGGQNHHEVVKFMDVYQRSYCHPIETLVDIFQEY
PDEIEYIFKPSCVPLMRCGGCCNAAGLACVPTEESNITMQ
IMRIKPHQGQHIGEMSFLQHNKCECRPKKDRARQEKCDK
PRR
Avi-tag, SEQ ID No: 26
GLNDIFEAQKIEWHE
ybbR-tag, SEQ ID No: 27
DSLEFIASKLA
FGE-tag, SEQ ID No: 28
LCTPSR
Cys-tag, SEQ ID No: 29
C
VEGF₁₂₁ sense nucleotide primer,
SEQ ID No: 30
catatggcacccatggcagaaggagga
VEGF₁₂₁ anti-sense nucleotide primer,
SEQ ID No: 31
ctcgagtcagtggtgatgatggtgatgggatccccgcctcggcttgtcac
VEGF_{DEE} flanking sense nucleotide primer,
SEQ ID No: 32
ggcacacatgccaggcctgctgcattgcagc
VEGF_{DEE} flanking anti-sense nucleotide primer,
SEQ ID No: 33
gtgcaatgcagcaggcctggcatgtgtgcc
VEGF₃₋₁₂₁-Avi fusion protein sense
nucleotide primer, SEQ ID No: 34
ccgagttaatgatgatgatgatgatgggatccttcgtgccattcgatttt
ctgagcctcgaagatgtcgttcagaccggatccccgcctcggcttg
VEGF₃₋₁₂₁-Avi fusion protein anti-sense
nucleotide primer, SEQ ID No: 35
ccgagttaatgatgatgatgatgatgggatccttcgtgccattcgataga
ctgagcctcgaagatgtcgttcagaccggatccccgcctcggcttg

We claim the following:

1. A polypeptide comprising:
a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGF121-_{DEE} Mutant (SEQ ID No: 8), VEGFR-2 specific mutant VEGF_{DEE} (SEQ ID No: 8), VEGFR-1 specific mutant VEGF_{NLS}(SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label.

2. The polypeptide of claim 1, wherein the VEGF protein is selected from:
VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), and VEGF₂₀₆ protein (SEQ ID No: 7).

3. The polypeptide of claim 1, wherein the VEGF protein is VEGF₁₂₁ protein (SEQ ID No: 1).

4. The polypeptide of claim 1, wherein the label is ¹⁸F.

5. The polypeptide of claim 1, wherein VEGF protein is VEGF₁₂₁ protein (SEQ ID No: 1), wherein the label is linked to the VEGF₁₂₁ protein with a chelator.

6. The polypeptide of claim 5, wherein the chelator is a macrocyclic chelator.

7. The polypeptide of claim 6, wherein the macrocyclic chelator is 1, 4, 7, 10-tetraazadodecane-N,N′,N″, N′″-tetraacetic acid (DOTA).

8. The polypeptide of claim 7, wherein the label is ⁶⁴Cu.

9. The polypeptide of claim 1, wherein the VEGF protein is VEGF_{DEE} Mutant (SEQ ID No: 8).

10. A kit for imaging comprising:
a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS}(SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and directions for use.

11. A method for imaging tissue, comprising:
contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS}(SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label, and imaging the tissue with a PET imaging system.

12. The method of claim 11, wherein the imaging is performed in vivo.

13. The method of claim 11, wherein tissue is selected from: tissue containing precancerous cells, cancer tissue, and/or tumor tissue.

14. The method of claim 11, wherein the tissue is selected from: cells related to ischemic or hypoxic related diseases.

15. The method of claim 11, wherein labeled VEGF protein is specific for VEGFR-1.

16. The method of claim 11, wherein labeled VEGF protein is specific for VEGFR-2, wherein the labeled VEGF protein is VEGF_{DEE} Mutant (SEQ ID No: 8).

17. A method of diagnosing the presence in a tissue of one or more of precancerous cells, cancerous cells, tumor cells, and cells related to ischemic or hypoxic related diseases, comprising:
contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE}(SEQ ID No: 8), VEGFR-1 specific mutant VEGF_{NLS}(SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and imaging the tissue with a PET imaging system.

18. A method of monitoring the progression in a tissue of the presence of one or more of tissue containing precancerous cells, tissue containing cancerous cells, tissue containing tumor cells, and tissue containing cells related to ischemic or hypoxic related diseases, comprising:
contacting a tissue with a labeled VEGF protein, wherein the VEGF protein is selected from: VEGF₁₂₁ protein (SEQ ID No: 1), VEGF₁₄₅ protein (SEQ ID No: 2), VEGF₁₄₈ protein (SEQ ID No: 3), VEGF₁₆₅ protein (SEQ ID No: 4), VEGF₁₈₃ protein (SEQ ID No: 5), VEGF₁₈₉ protein (SEQ ID No: 6), VEGF₂₀₆ protein (SEQ ID No: 7), VEGFR-2 specific mutant VEGF_{DEE} (SEQ ID No: 8), VEGFR-1 specific mutant VEGF_{NLS} (SEQ ID No: 9), VEGF₃₋₁₂₁-Avi (SEQ ID No: 10), Avi-VEGF₃₋₁₂₁ (SEQ ID No: 11), VEGF_{DEE}-Avi (SEQ ID No: 12), Avi-VEGF_{DEE} (SEQ ID No: 13), VEGF₃₋₁₂₁-ybbR (SEQ ID No: 14), ybbR-VEGF₃₋₁₂₁ (SEQ ID No: 15), VEGF_{DEE}-ybbR (SEQ ID No: 16), ybbR-VEGF_{DEE} (SEQ ID No: 17), VEGF₃₋₁₂₁-FGE (SEQ ID No: 18), FGE-VEGF₃₋₁₂₁ (SEQ ID No: 19), VEGF_{DEE}-FGE (SEQ ID No: 20), FGE-VEGF_{DEE} (SEQ ID No: 21), VEGF₃₋₁₂₁-Cys (SEQ ID No: 22), Cys-VEGF₃₋₁₂₁ (SEQ ID No: 23), VEGF_{DEE}-Cys (SEQ ID No: 24), and Cys-VEGF_{DEE} (SEQ ID No: 25), wherein the label is a PET radioisotope label; and imaging the tissue with a PET imaging system.


SEQUENCE LISTING (35 sequences)

SEQ ID NO: 1 - protein, 121 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 2 - protein, 145 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 3 - protein, 148 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 4 - protein, 165 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - protein, 183 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 6 - protein, 189 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 7 - protein, 206 aa, Homo sapien
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 8 - protein, 124 aa, Homo sapien
MAPMAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRRGS

SEQ ID NO: 9 - protein, 122 aa, Homo sapien
MAPMAEGGGQNHHEVVKFMDVYQRSYCHPIETLVDIFQEYPDEIEYIFKPSCVPLMRCGGCCNDEGLECVPTEESNITMQIMNLSPHQGQHIGEMSFLQHNKCECRPKKDRARQEKCDKPRR

SEQ ID NO: 10 - protein, 136 aa, Homo sapien
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRGSGLNDIFEAQKIEWHE

SEQ ID NO: 11 - protein, 136 aa, Homo sapien
MGLNDIFEAQKIEWHEGSAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 12 - protein, 136 aa, Homo sapien
MAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRRGSGLNDIFEAQKIEWHE

SEQ ID NO: 13 - protein, 136 aa, Homo sapien
MGLNDIFEAQKIEWHEGSAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRR

SEQ ID NO: 14 - protein, 132 aa, Homo sapien
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRGSDSLEFIASKLA

SEQ ID NO: 15 - protein, 132 aa, Homo sapien
MDSLEFIASKLAGSAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 16 - protein, 132 aa, Homo sapien
MAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRRGSDSLEFIASKLA

SEQ ID NO: 17 - protein, 132 aa, Homo sapien
MDSLEFIASKLAGSAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRR

SEQ ID NO: 18 - protein, 127 aa, Homo sapien
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRGSLCTPSR

SEQ ID NO: 19 - protein, 127 aa, Homo sapien
MLCTPSRGSAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 20 - protein, 127 aa, Homo sapien
MAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRRGSLCTPSR

SEQ ID NO: 21 - protein, 127 aa, Homo sapien
MLCTPSRGSAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRR

SEQ ID NO: 22 - protein, 122 aa, Homo sapien
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRGSC

SEQ ID NO: 23 - protein, 121 aa, Homo sapien
CGSAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 24 - protein, 122 aa, Homo sapien
MAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRRGSC

SEQ ID NO: 25 - protein, 121 aa, Homo sapien
CGSAEGGGQNHHEVVKFMDVYQRSYCHPIETLVDIFQEYPDEIEYIFKPSCVPLMRCGGCCNAAGLACVPTEESNITMQIMRIKPHQGQHIGEMSFLQHNKCECRPKKDRARQEKCDKPRR

SEQ ID NO: 26 - protein, 15 aa, Homo sapien
GLNDIFEAQKIEWHE

SEQ ID NO: 27 - protein, 11 aa, Homo sapien
DSLEFIASKLA

SEQ ID NO: 28 - protein, 6 aa, Homo sapien
LCTPSR

SEQ ID NO: 29 - protein, 1 aa, Artificial Sequence
  Primer sequence
C

SEQ ID NO: 30 - DNA, 27 bp, Artificial Sequence
  Primer sequence
catatggcacccatggcagaaggagga

SEQ ID NO: 31 - DNA, 50 bp, Artificial Sequence
  Primer Sequence
ctcgagtcagtggtgatgatggtgatgggatccccgcctcggcttgtcac

SEQ ID NO: 32 - DNA, 31 bp, Artificial Sequence
  Primer sequence
ggcacacatgccaggcctgctgcattgcagc

SEQ ID NO: 33 - DNA, 30 bp, Artificial Sequence
  Primer Sequence
gtgcaatgcagcaggcctggcatgtgtgcc

SEQ ID NO: 34 - DNA, 96 bp, Artificial Sequence
  Primer Sequence
ccgagttaatgatgatgatgatgatgggatccttcgtgccattcgattttctgagcctcgaagatgtcgttcagaccggatccccgcctcggcttg

SEQ ID NO: 35 - DNA, 96 bp, Artificial Sequence
  Primer Sequence
ccgagttaatgatgatgatgatgatgggatccttcgtgccattcgatagactgagcctcgaagatgtcgttcagaccggatccccgcctcggcttg